(19) United States
(12) Patent Application Publication
(10) Pub. No.: US 2025/0195713 A1
(43) Pub. Date: Jun. 19, 2025

(54) SYSTEMS AND METHODS FOR AUTOMATIC AIR PATHOGEN MITIGATION

(71) Applicant: UV Partners, Inc., Grand Haven, MI (US)

(72) Inventors: David W Baarman, Fennville, MI (US); Colin J. Moore, Grand Rapids, MI (US); Ryan D. Schamper, Grand Haven, MI (US); Michael Halloran, Hudsonville, MI (US); Jacob Herbert, Grand Haven, MI (US); Joseph C. Van Den Brink, Coopersville, MI (US)

(21) Appl. No.: 18/846,528

(22) PCT Filed: Mar. 17, 2023
(86) PCT No.: PCT/US2023/015449

§ 371 Date: Sep. 12, 2024

Related U.S. Application Data
(60) Provisional application No. 63/320,967, filed on Mar. 17, 2022.

(51) Int. Cl. A61L 9/20

(52) U.S. Cl. CPC A61L 9/20; A61L 2209/111; A61L 2209/12; A61L 2209/15

(57) ABSTRACT

A system for tracking the source of pathogens with a variable response and mitigation method utilizing a combination of UV, filtration, fans, communications and a control system. The control system monitors sensors that are indicative of pathogen propagation creating triggers and thresholds for a known measured response. The UV air treatment systems can generally include one or more UV air treatment fixtures as well as systems and methods for installing, operating, and maintaining the one or more UV air treatment fixtures. Some aspect emphasize features and configurations related to installation, operation, and maintenance of individual UV air treatment fixtures, while other aspects emphasize features and configurations related to coordination of multiple air treatment systems including data collection and control.

### FIELD OF THE INVENTION

The present disclosure relates to air pathogen mitigation, and more particularly toward automatically reducing pathogens based on environmental information and sensors while saving energy, enhancing filter life and lamp life, and directing UV energy in a suitable fashion.

### BACKGROUND OF THE INVENTION

Embodiments of the present disclosure relate to general installation, operation, monitoring, and maintenance of air treatment ceiling fixtures with pathogen mitigation functionality. Some embodiments of the present invention also relate to sensors, detection, and dynamic control based on same.

Infection by a foreign organism, such as bacteria, viruses, fungi, or parasites, can be acquired in a variety of ways. But once acquired, the infection, if harmful, may colonize and result in illness. The immune system of the infected host (e.g., the person) may react to the infection and attempt to kill or neutralize the foreign organism. However, in some cases, the immune system may be insufficient to completely neutralize the infection. For these and other reasons, infectious disease prevention is conventionally preferred over reliance solely on the immune system of the infected host.

Conventional efforts to prevent spread of infectious disease often involve manual disinfection techniques, such as wiping down or washing surfaces that may harbor foreign organisms. Because infectious diseases can be spread in a variety of ways, such as via direct contact from person to person, manual disinfection techniques can be time and labor intensive. Air borne pathogens from an infected person can make their way into areas that are inaccessible to manual disinfection techniques. It is also known that contact pathogens can be airborne on the typical airborne particulates.

The room environments, such as hospital rooms, include air and surfaces that can become contaminated. It can be labor intensive to manually decontaminate such environments due to the volume of air and the number and variety of surfaces (e.g., nooks and crannies created by presence of objects in the room). The HVAC system for a room is particularly labor intensive to decontaminate and is typically mixing and distributing particulates. Additionally, or alternatively, in hospital environments (e.g., a patient room), the number and frequency of visitors and potential pathogens increases the likelihood of air and surface contamination, again increasing the labor and time to effectively decontaminate the room with conventional techniques. For these and other reasons, conventional techniques fail to enable decontamination of room environments in a practical manner.

Conventional disinfection techniques for hospital rooms involve transporting a mobile UV lighting assembly in the room. The mobile UV lighting assembly is positioned within the room and activated for a period of time considered sufficient to disinfect the room. The mobile UV lighting assembly is then removed from the room and transported to storage or to another room for use. This process can be laborious due to the effort to transport and move the assembly and the effort to track a schedule for use of the assembly across several rooms.

Another disinfection technique for hospital rooms is air treatment ceiling units. Known air treatment ceiling units generally treat volumes of air indiscriminately. These systems are generally configured based on an assumed air exchange per hour referring to the air in the whole environment being exchanged while in actuality only portions of the air may be exchanged. Any obstructions, such as office furniture, partitions, cabinets and other equipment can trap air and create pressures, vortices, and turbulence within airflows that affects the air exchange. This further complicates indiscriminately treating airflows. Many air treatment ceiling units lack the capability to understand the local pathogen load or any way in which to tailor local pathogen load reduction. Many current air treatment ceiling systems require 24/7 operation. Many current air treatment systems lack the understanding of basic consumption data. Many air treatment ceiling systems are simple and monitor little or no stimulus while others may have basic proximity sensing that control crude on/off controls.

Air treatment ceiling fixtures often are installed in a drop ceiling. Drop ceilings are configured with a variety of cell sizes including 2 by 4 foot cells and 2 by 2 foot cells. Many current air treatment ceiling fixtures are too large to fit within drop ceiling that cells and those that do, generally cannot accommodate smaller footprint cells, such as a 2 by 2 feet cells.

Further, the variety of cell sizes presents a challenge in making an air treatment ceiling fixture with a single footprint that can be used across multiple ceiling configurations, both in how the air treatment ceiling fixture can be mounted within the cell and how to make the air treatment ceiling fixture(s) aesthetically pleasing in relation to the rest of the drop ceiling and any other air treatment ceiling fixtures. In addition to a small cell footprint, drop ceilings have limited plenum space between the ceiling T-rail system and the true ceiling. This makes installation of air treatment ceiling fixtures challenging because there is limited room within the ceiling to maneuver the air treatment ceiling fixture within the ceiling to install it. Limited plenum space makes it more difficult to access the air treatment ceiling fixture for maintenance after it has been installed in the ceiling.

### SUMMARY OF THE INVENTION

This disclosure provides a number of solutions to problems with air treatment ceiling units and air treatment systems including multiple air treatment ceiling units. A number of problems have been observed and the UV air treatment fixtures of the present disclosure provide improvements.

UV air treatment ceiling units in accordance with the present disclosure strike a suitable balance between air flow through the UV reactor chamber, UV reflection, and the size of the UV reactor chamber to provide effective air treatment in a small footprint. The UV air treatment ceiling fixtures of the present disclosure include UV reducer airflow directors that simultaneously reduce the amount of UV light and permit airflow.

The UV air treatment fixtures of the present disclosure can provide area treatment within an environment to mitigate source control within that space. Multiple UV air treatment fixtures can be operated in conjunction to enhance pathogen mitigation. The UV air treatment fixtures can include various monitoring methods, sensors, as well as resolution and event tracking to automatically determine and select a suitable treatment mode and level of operation. The UV air treatment fixtures of the present disclosure can include a variety of sensors both for monitoring operation, but also monitoring events in standby or low power mode so that the system can conserve energy, but be configured for switching modes when interrupted by certain control triggers, such as air flow changes, HVAC events, or the like to provide a treatment period with specific time based on pathogen settling times.

Some aspects of the present disclosure emphasize features of an air treatment ceiling unit that improves installation, configurability, and aesthetics. Some air treatment ceiling units can be configured in accordance with the present disclosure for US or metric installations, provide a sustainable air treatment solution with measurable outcomes. Some embodiments can include components that provide an ease of configuration and versatility of use. Further, some embodiments can enable connecting use data to the cloud for machine learning or other forms of analysis. By monitoring the air treatment ceiling unit use and other data, the programming of the system can be updated using over the air programming, which can lead to an improved configuration that provides improved outcomes.

These and other advantages and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components. Any reference to claim elements as “at least one of X, Y and Z” is meant to include any one of X, Y or Z individually, and any combination of X, Y and Z, for example, X, Y, Z; X, Y; X, Z; and Y, Z.

### BRIEF DESCRIPTION OF THE DRAWINGS

FIG. 1 illustrates a representational view of airflow through an air treatment reactor configuration according to one embodiment.

FIG. 2A illustrates a representational view of airflow through an air treatment reactor configuration according to one embodiment.

FIG. 2B illustrates a representational view of airflow through an air treatment reactor configuration according to one embodiment.

FIG. 3 illustrates a representational view of airflow through an air treatment reactor configuration according to one embodiment.

FIG. 4A illustrates a bottom perspective view of a portion of an air treatment ceiling system according to one embodiment with the UV reactor chamber access door open.

FIG. 4B illustrates a close-up view of a UV airflow director of the FIG. 4A air treatment ceiling system.

FIG. 4C illustrates a bottom perspective view of the UV air treatment ceiling system of FIG. 4A.

FIG. 5 illustrates a representational view of airflow through an air treatment reactor configuration according to one embodiment.

FIG. 6A illustrates a partial sectional view showing a hinge for a configurable maintenance door of an air treatment ceiling system according to one embodiment.

FIG. 6B illustrates an alternative hinge configuration according to one embodiment.

FIG. 6C illustrates an alternative hinge configuration according to one embodiment.

FIG. 7 illustrates a ceiling integration system for an air treatment ceiling system according to one embodiment.

FIGS. 8A-E illustrate a ceiling integration system for an air treatment ceiling system according to one embodiment.

FIG. 9A illustrates a deployable mounting system for an air treatment ceiling system according to one embodiment.

FIG. 9B illustrates a ceiling integration system for use with the air treatment ceiling system of FIG. 9A according to one embodiment.

FIG. 10 illustrates a ceiling integration system for an air treatment ceiling system according to one embodiment.

FIG. 11 illustrates a ceiling integration system for an air treatment ceiling system according to one embodiment.

FIG. 12A illustrates a ceiling integration system for an air treatment ceiling system according to one embodiment.

FIG. 12B illustrates an air treatment ceiling system corresponding to FIG. 12A.

FIG. 12C illustrates the ceiling integration system of FIG. 12A installed in the air treatment ceiling system of FIG. 12B.

FIG. 13A illustrates a ceiling integration system for an air treatment ceiling system according to one embodiment.

FIG. 13B illustrates the air treatment ceiling system corresponding to FIG. 13A.

FIG. 13C illustrates the ceiling integration system of FIG. 13A installed in the air treatment system of FIG. 13B.

FIGS. 14A-D illustrate an air treatment ceiling system according to one embodiment installed on four different ceiling T-rail systems.

FIG. 15 illustrates a ceiling integration system for an air treatment ceiling system according to one embodiment.

FIGS. 16A-D respectively illustrate cross-sections of the air treatment ceiling systems installed on the four different ceiling T-rail systems of FIGS. 14A-D.

FIG. 17 illustrates a perspective view an air treatment ceiling system with a configurable maintenance door according to one embodiment.

FIGS. 18A-D illustrate configurable maintenance doors according to various embodiments.

FIGS. 19A-D illustrate configurable maintenance doors according to various embodiments.

FIG. 20 illustrates an air treatment ceiling system user interface device according to one embodiment.

FIG. 21 illustrates a table-mountable air treatment system according to one embodiment.

FIG. 22 illustrates the air treatment system of FIG. 21 mounted to a table.

FIG. 23 illustrates a top perspective view of the table of FIG. 22 with the air treatment system mounted underneath.

FIG. 24 illustrates an exemplary control system for an air treatment ceiling system in accordance with the present disclosure.

FIG. 25 illustrates a top perspective view of an air treatment ceiling system with an electronic cover panel removed according to one embodiment.

FIG. 26 illustrates a connected pathogen reduction system controlling and powering multiple air pathogen reduction hardware systems in accordance with one embodiment.

FIG. 27 illustrates a connected pathogen reduction system in accordance with one embodiment.

FIG. 28A illustrates a top view of an air treatment ceiling system according to one embodiment.

FIG. 28B illustrates a sectional view of the air treatment ceiling system of FIG. 28A along the line 28B.

FIG. 29A illustrates a top view of an air treatment ceiling system according to one embodiment.

FIG. 29B illustrates a sectional view of the air treatment ceiling system of FIG. 29A along the line 29B.

FIG. 30A is a bottom view of an air treatment ceiling system according to one embodiment.

FIG. 30B is a top view of the air treatment ceiling system of FIG. 30A.

FIG. 30C is a sectional view of the air treatment ceiling system of FIG. 30B along the line 30C.

FIG. 30D illustrates a close-up portion of detail 30D of FIG. 30C.

FIG. 30E illustrates a close-up portion of detail 30E of FIG. 30C.

FIG. 31 illustrates a sectional view of an air treatment ceiling system without a visible lighting element in the configurable maintenance door.

FIG. 32 illustrates a bottom view of an air treatment ceiling system according to one embodiment.

FIG. 33 illustrates a sectional view of the air treatment ceiling system of FIG. 32 along the line 33.

FIG. 34 illustrates a sectional view of the air treatment ceiling system of FIG. 33 along the line 34.

FIG. 35A illustrates a top view of an air treatment ceiling system according to one embodiment.

FIG. 35B illustrates a side view of the air treatment ceiling system of FIG. 35A.

FIG. 35C illustrates a sectional view of the air treatment ceiling system of FIG. 35A along the line 35C.

FIG. 36 illustrates a bottom view of an air treatment ceiling system having latches according to one embodiment.

FIG. 37 illustrates a bracket for holding UV absorbing air directors according to one embodiment.

FIG. 38 illustrates a representative building layout of air treatment ceiling systems for collecting environmental health data and mitigating pathogen exposure.

FIG. 39 illustrates an exemplary graph of quanta of pathogens as it relates to the number of people changing in a room.

FIG. 40 illustrates an exemplary graph illustrating fan speed controlled based on quanta of pathogens as the number of people changes in a room.

FIG. 41 shows an exemplary graph for coordinating multiple air treatment ceiling systems to provide a desired ambient sound level.

FIG. 42 illustrates an exemplary flowchart for a sound and people calibration process for initial system set up and configuration according to one embodiment.

FIG. 43 illustrates an exemplary operational flow chart for coordinating operation and data gathering of multiple air treatment ceiling systems according to one embodiment.

FIG. 44 illustrates an exemplary sensor data daily table.

FIG. 45 illustrates a bottom perspective view of another embodiment of a UV air treatment system installed under a table.

FIG. 46 illustrates an exploded view of the table of FIG. 45.

FIG. 47 illustrates a top view of the table of FIG. 45.

FIG. 48 illustrates a perspective view of a portion of the UV air treatment system of FIG. 45.

FIG. 49 illustrates a top view of the UV air treatment system of FIG. 48.

FIG. 50 illustrates an exploded view of the UV air treatment system of FIG. 48.

FIG. 51 illustrates a sectional view of the UV air treatment system of FIG. 49 cut along line 51.

FIG. 52 illustrates a top perspective view of an installed UV air treatment body just before a configurable maintenance door is about to be installed.

FIG. 53 illustrates a close-up view of one corner of the UV air treatment body and configurable maintenance door of FIG. 52.

FIG. 54 illustrates a top perspective view of the fully installed UV air treatment ceiling fixture of FIG. 52.

FIG. 55 illustrates a close-up view of one corner of the UV air treatment body and configurable maintenance door of FIG. 54.

FIGS. 56A-B illustrate an installation sequence for installing a configurable maintenance door to a UV air treatment body already installed in a ceiling.

FIG. 57 illustrates a bottom perspective view of a UV treatment body just before installation into a ceiling.

FIG. 58-64 illustrate a sequence of bottom perspective views of the UV treatment body of FIG. 57 being installed into a ceiling.

FIG. 65 illustrates a bottom perspective view of a UV treatment ceiling fixture installed in a ceiling after a configurable maintenance door is installed to the UV air treatment body.

FIG. 66 illustrates an angled UV airflow director in accordance with one embodiment.

FIG. 67 illustrates a top view of the angled UV airflow director of FIG. 66.

FIG. 68 illustrates a sectional view of the angled UV airflow director of FIG. 67 cut along line 68.

FIG. 69 illustrates a curved UV airflow director in accordance with one embodiment.

FIG. 70 illustrates a top view of the curved UV airflow director of FIG. 69.

FIG. 71 illustrates a sectional view of the angled UV airflow director of FIG. 70 cut along line 71.

FIG. 72 illustrates a v-shaped UV airflow director in accordance with one embodiment.

FIG. 73 illustrates a top view of the v-shaped UV airflow director of FIG. 72.

FIG. 74 illustrates a sectional view of the v-shaped UV airflow director of FIG. 73 cut along line 74.

FIG. 75 illustrates an exploded view of a configurable maintenance door with a downlight in accordance with one embodiment.

FIG. 76 illustrates a bottom view of the configurable maintenance door with downlight of FIG. 75.

FIG. 77 illustrates a partial sectional view of the configurable maintenance door with downlight of FIG. 76 along line 77.

FIG. 78 illustrates an exploded view of a configurable maintenance door with a reversible aesthetic mounting panel in accordance with one embodiment.

FIG. 79 illustrates a bottom view of the configurable maintenance door of FIG. 78.

FIG. 80 illustrates a partial sectional view of the configurable maintenance door of FIG. 78 along line 80.

FIG. 81 illustrates an upper perspective view of an optional locking tab engaging a t-rail wall in the plenum of the ceiling.

### DESCRIPTION OF THE CURRENT EMBODIMENTS

The present disclosure is generally directed to various aspects of UV air treatment. The UV air treatment systems of the present disclosure can generally include one or more UV air treatment fixtures as well as systems and methods for installing, operating, and maintaining the one or more UV air treatment fixtures. Some aspects emphasize features and configurations related to installation, operation, and maintenance of individual UV air treatment fixtures, while other aspects emphasize features and configurations related to coordination of multiple air treatment systems including data collection and control.

Many embodiments of the present disclosure relate to UV air treatment ceiling fixtures that can be installed within a cell of a drop or grid ceiling (e.g., a ceiling with a t-rail system). Other embodiments can be installed in a hard ceiling with an optional mounting kit. The present disclosure provides various combinations of features that enable scaling air treatment ceiling systems to a smaller footprint (e.g., a 2 by 2 foot cell instead of a 2 by 4 foot cell) while providing increased performance, such as more effective pathogen mitigation than conventional UV air treatment ceiling fixtures (e.g., one installable in a 2 by 4 foot cell that has a larger UV reactor chamber). Air treatment ceiling units in accordance with the present disclosure strike a suitable balance between air flow through the UV reactor chamber, UV reflection, and the size of the UV reactor chamber to provide effective air treatment in a small footprint. A 2 by 4 foot cell UV air treatment fixture is disclosed in WO2021/138645 entitled System and Method of Disinfection to Baarman et al., filed on Jan. 2, 2021 is directed to a fixture for disinfecting air within a room, and is hereby incorporated by reference in its entirety.

This disclosure relates to a number of different aspects of air treatment ceiling fixtures. Some examples include deployable mounting systems for installing the UV air treatment ceiling fixture, UV reactor chamber configurations for mitigating pathogens in the air routed through the UV reactor chamber, UV air flow director configurations for directing airflow to and from a UV reactor chamber at a suitable airflow velocity while preventing an unsatisfactory amount of UV light from escaping the UV air treatment ceiling fixture, configurable maintenance door configurations for sealing the UV reactor chamber in a closed position and providing maintenance access to the various components of the UV air treatment ceiling fixture in an open position, ceiling integration systems for providing a suitable aesthetic appearance relative to the ceiling, to name a few.

A system and method in accordance with one embodiment may include a UV air treatment fixture configured to be disposed within a room and provide air pathogen mitigation via application of UV light to air flowing through an air treatment chamber, also referred to as a UV reactor chamber. In one embodiment, one or more UV light directors may be disposed within the chamber to simultaneously allow airflow while preventing UV light from leaking past the one or more UV reducing airflow directors into the room.

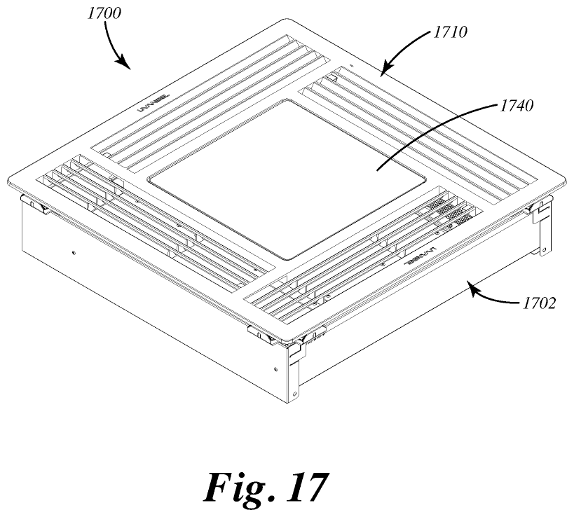

A UV air treatment ceiling fixture in accordance with one embodiment of the present disclosure is shown in FIG. 17 and generally designated 1700. The fixture is configured for installation within a drop ceiling via a ceiling integration system, which is discussed in more detail in later sections. The fixture 1700 may receive power from an external power source, and may be connected to the external power source in a variety of ways depending on the application, such as by direct wiring or via connection to an outlet socket. The fixture 1700 in one embodiment may include a control system (See e.g., control system 2400 in FIG. 24) configured to control operation of the fixture 1700 and components thereof, which will be discussed in more detail in later sections. The UV air treatment ceiling fixture includes two main parts, the UV air treatment body 1702 and the maintenance door or grille 1710, which are each discussed in more detail below.

### Installation

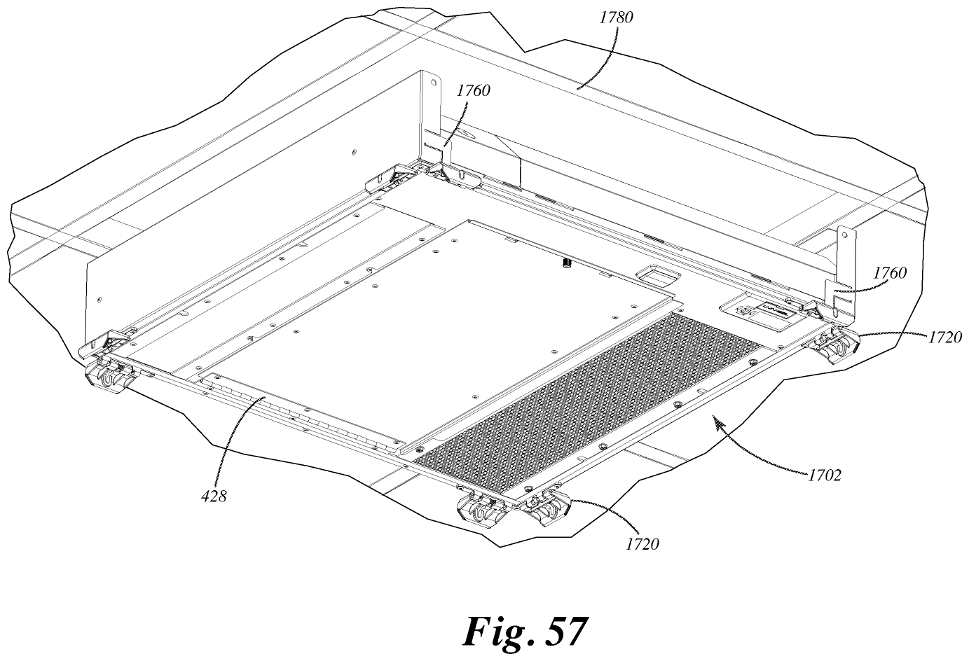

Installation begins with the UV air treatment body 1702 and will now be discussed in detail with reference to FIGS. 57-64. Perhaps as best shown in FIG. 57, in the current embodiment, each of the bottom corners of the UV air treatment body 1702 has a pair of vertical installation clip assemblies 1720 that cooperate to mount the UV air treatment body 1702 in a grid ceiling. That is, in its installed state, the vertical installation clip assemblies 1720 engage the top of the ceiling grid T-rail 1780 and cooperatively support the UV air treatment body 1702 in the plenum of the ceiling.

In total, the current embodiment includes eight vertical clip assemblies 1720. However, in alternative embodiments, the number and placements of the clip assemblies 1720 can vary. For example, in some embodiments two, three, four, five, six, or seven vertical installation clip assemblies may be sufficient to cooperatively support the UV air treatment body 1720 on the T-rail grid 1780. In other embodiments, more than eight vertical clip assemblies may be provided.

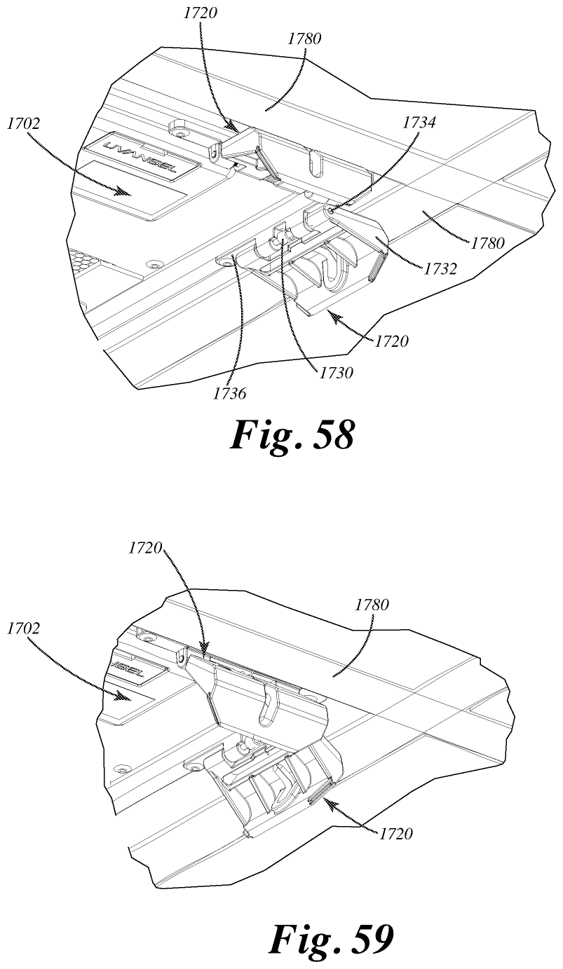

Perhaps as best shown in FIG. 58, in the current embodiment, each vertical installation clip assembly 1720 includes a vertical installation clip 1732, a torsion spring 1730, a knurled pin 1734, and a vertical installation clip bracket 1736. In the current embodiment, the brackets 1736 are mounted about the perimeter of the bottom face of the UV air treatment body 1702. The pin 1734 holds the assembly together and the spring 1730 in place by slotting through apertures in the bracket 1736 and the clip 1732. With appropriate positioning and orientation the torsion spring provides a resting state that where the clip is rotated outside the perimeter of the UV air treatment body 1702. As downward force is applied to the clip 1732 and it is displaced (e.g. as the installation is lifted into the ceiling and the back of the clip 1732 interfaces with the t-rail grid 1780), torque increases in the spring 1730. When the downward forces is removed (e.g., after the clip clears the t-rail grid), the tension in the spring releases and the clip rotates back to its original position outside the UV air treatment base perimeter where it can reach and rest upon the top of the T-rail grid 1780. Although the current embodiment of the vertical installation clips utilize a torsion spring, in alternative embodiments other clip rotation mechanisms can be utilized instead. Essentially any clip assembly that provides a resting state with the clip outside the UV air treatment base, but allows for deflection of the clip to within the bounds of the UV air treatment base can be suitable.

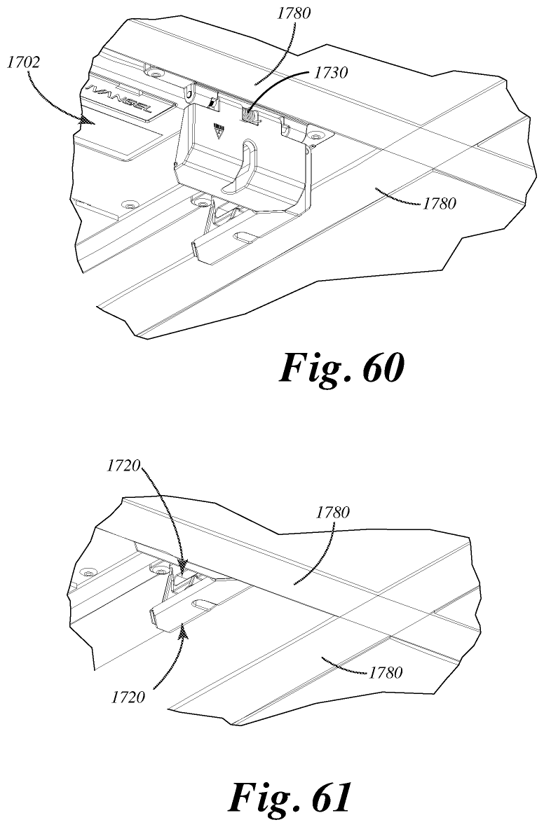

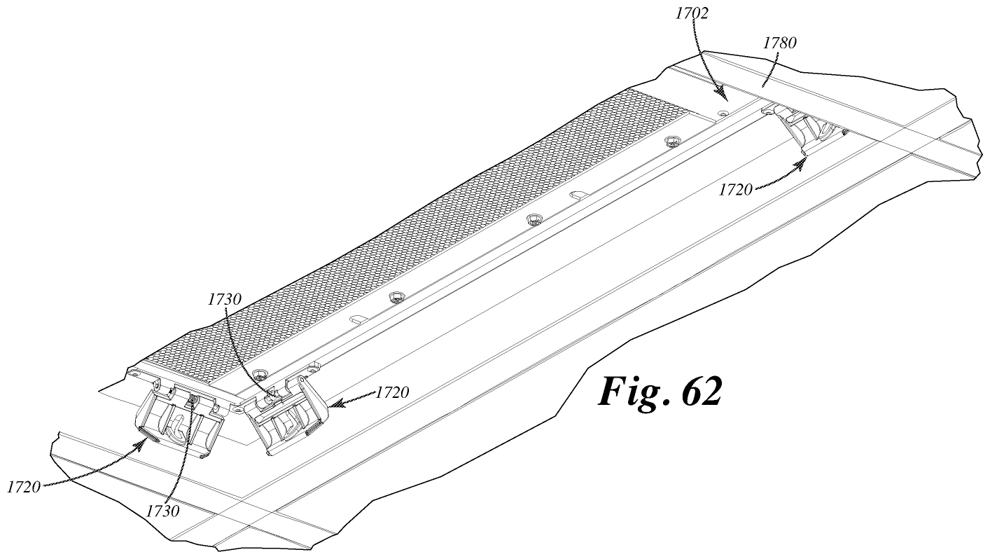

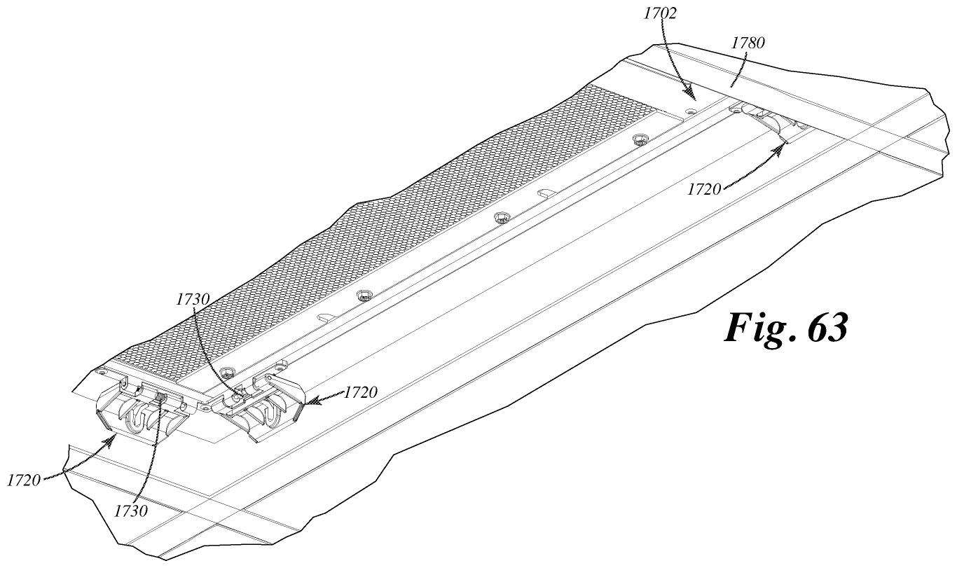

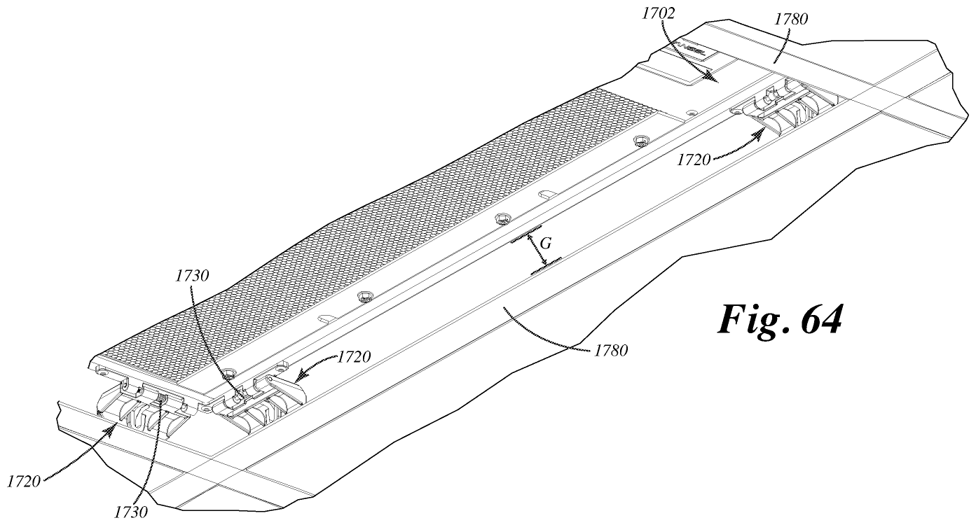

Referring to FIGS. 58-64, this sequence of figures depicts how the vertical installation clips react as the UV air treatment body 1702 is lifted into the grid ceiling. In FIG. 58, the back of each clip 1720 begins to interface with the UV air treatment body 1720. In FIG. 59, the back of each clip 1720 begins to deflect downward and toward the UV air treatment body 1720. In FIG. 60, the clips 1720 reach their full extension to a generally vertical position providing sufficient clearance for the UV air treatment body 1702. In FIG. 61, the UV treatment body is shown being lifted above the ceiling grid T-rail 1780 where there is no longer interference from the T-rail 1780. FIG. 62 illustrates how the vertical installation clips 1720 react by rotating back to their starting position due to the force provided by the springs 1730. In FIG. 63, the clips 1720 are fully extended back to their starting positions and are ready to engage the T-rail 1780. In FIG. 64, the installer lowers the UV air treatment body 1702 onto the grid T-rail 1780 where the clips fully support and mount the UV air treatment system 1702 in the grid ceiling. Perhaps as best shown in FIG. 63, the amount of vertical clearance into the plenum is low because the only space needed in the plenum of the ceiling is the height of the UV treatment base plus the height of the clip in its rotated vertical position which in total for this embodiment is about five inches or so. Other embodiments may be configured to use more or less clearance by adjusting the depth of the UV air treatment body and clip length.

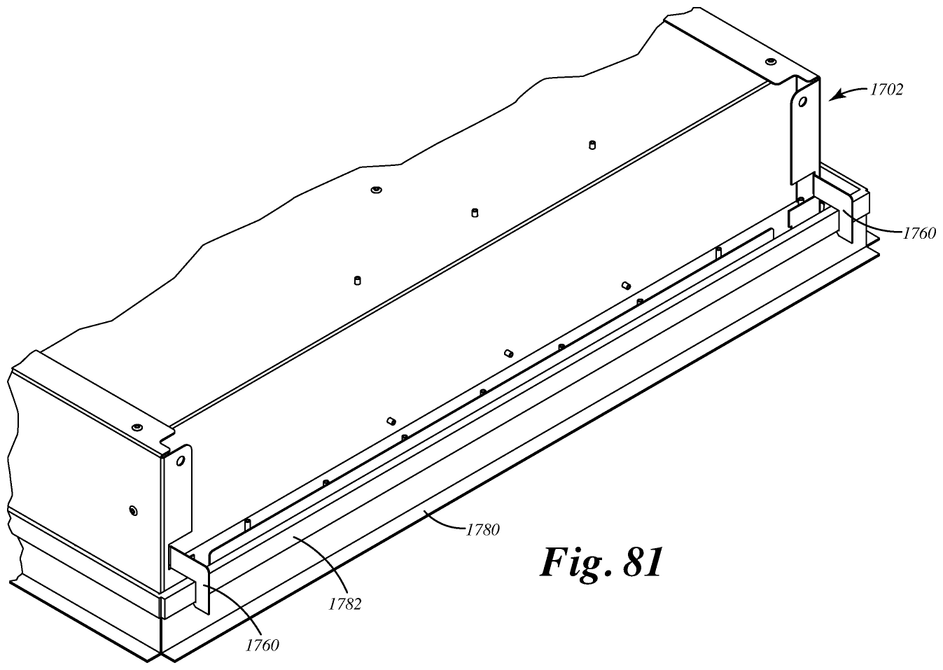

With the UV air treatment body 1702 suspended on the grid T-rail 1780 additional, optional, steps may be taken to further secure the UV air treatment body 1702. For example, a screwdriver may be utilized to insert self-piercing screws through a center hole of each (or some) of the vertical installation clip assemblies 1720 into the T-Grid vertical wall. Further, four grid-lock tabs 1760 are built into the housing. The installer can optionally bend each tab 1760 out about 90 degrees until it engages with the t-rail grid wall 1782 of the t-rail grid 1780 as shown in FIG. 81.

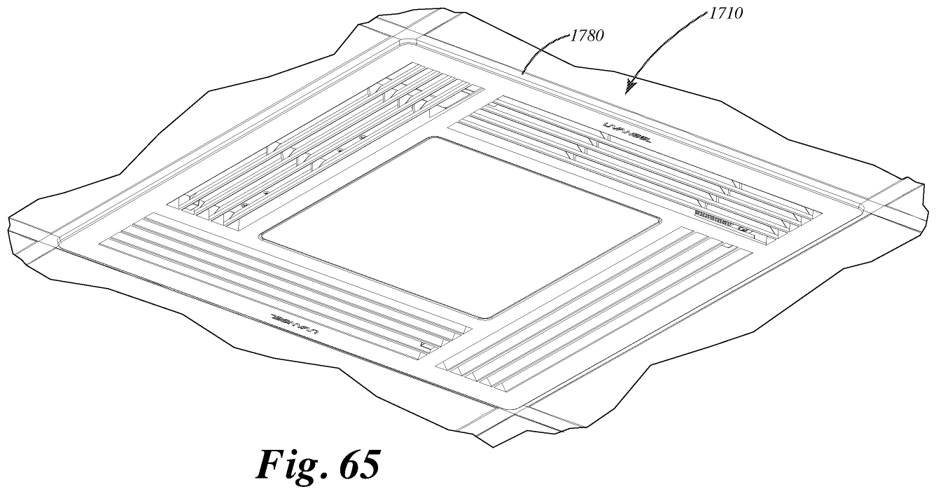

Once the UV air treatment body 1702 is installed in the ceiling there is a gap G between the perimeter of the UV air treatment system and the edge of the t-rail grid 1780. This gap can cause aesthetic and functional issues. Aesthetically, the gap G can be unpleasing to the eye disrupting the look and feel of the ceiling. Functionally, the gap G can permit airflow from the plenum to compete with airflow from the room below. To address both of these issues a grille 1710 or configurable maintenance door can be installed to the UV air treatment body 1702 as shown in FIG. 65 to cover the gap G when viewing from the ceiling below and seal the airflow from the plenum to the inlet of the UV air treatment body.

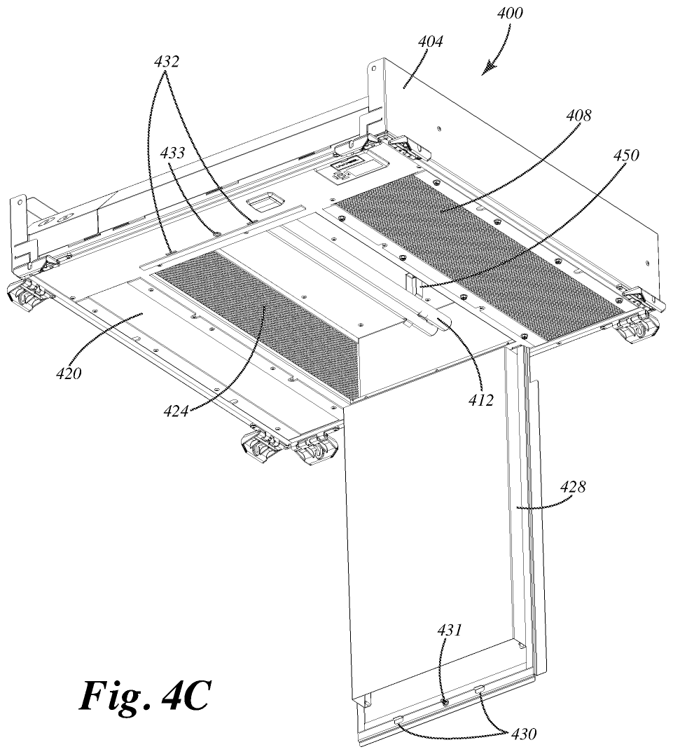

Before the grille is installed, UV-C reaction chamber lamp installation will be discussed in connection with FIGS. 4A and 4C. In these views, the UV reactor chamber access door 428 is depicted in its open state. The door 428 includes a hinge 429 (perhaps best shown in FIG. 57) at one end that allows the door to rotate between open and closed positions. It also includes a thumbscrew 431 and set of disconnect contacts 430. The thumbscrew can screw into the receiver 433 to hold the chamber access door shut. The disconnect contacts, when the door is in its seated position, protrude through the apertures 432 to make contact with the switches in the UV air treatment body electrical housing—this allows power to automatically shut off when the UV reactor chamber access door is opened.

Once the chamber access door 428 is lowered to its open position hanging from the fixture, a UV lamp can be inserted glass end through the retaining clip 450. From there, the UV lamp 412 base can be lined up and inserted into the lamp socket 413. Pressure can be applied until it clicks in to seat the lamp in the socket. After successful installation of the UV lamp, the chamber access door 428 can be closed with the thumbscrew. The replacement procedure for a UV lamp is the same.

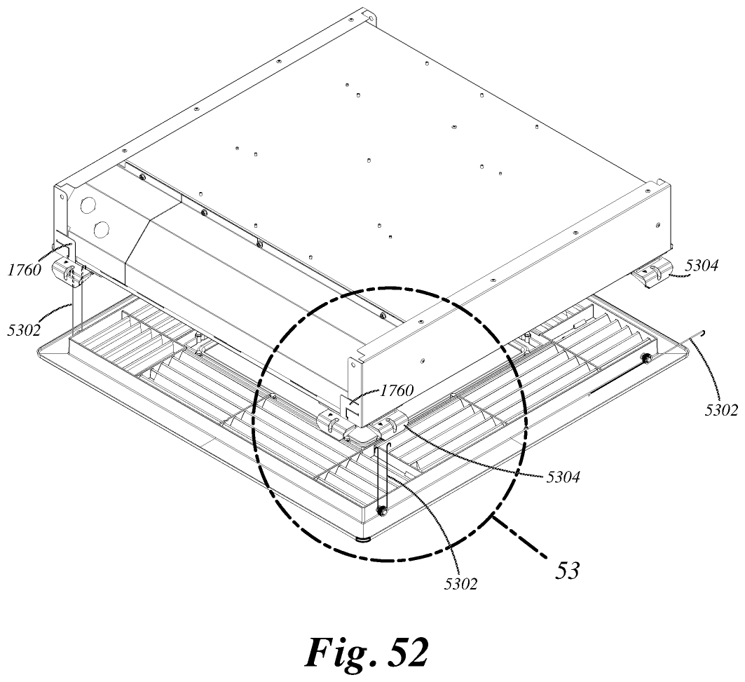

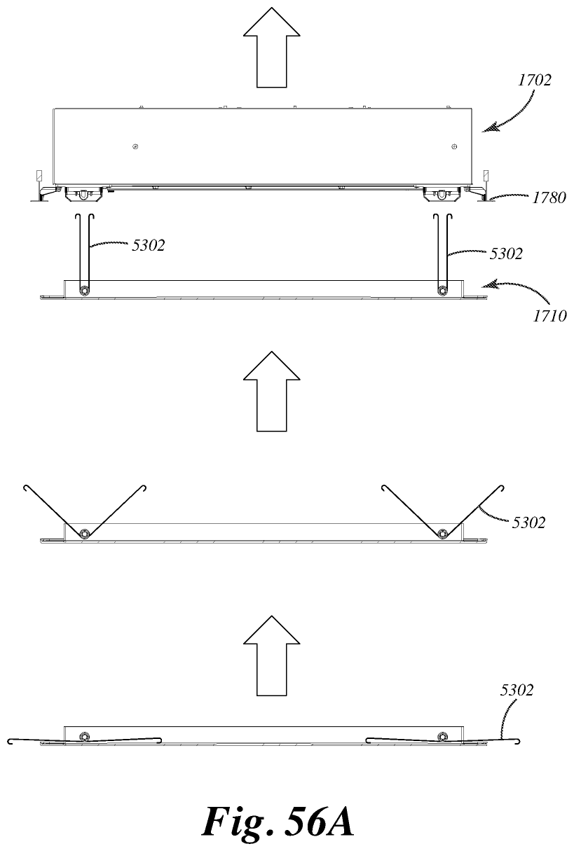

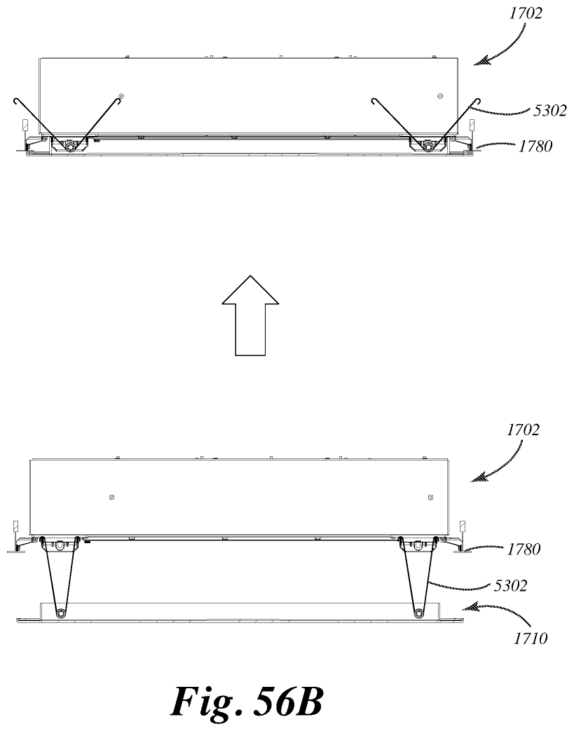

With the UV air treatment body 1720 mounted in the ceiling and the UV lamp installed, the grille 1710 is ready for installation, which will be discussed in connection with FIGS. 52-56. For installation, the ends of each grille retention springs 5302 are aligned and inserted into a respective vertical installation clip slot 5304 of a vertical installation clip. During installation the installer should assure the hooks at both ends of the spring 5302 are captured within the slot 5304. This process can be repeated for each retention spring 5302 to fully install the grille to the UV air treatment base 1702 as shown in the sequence diagram of FIG. 56A-B. FIG. 56A shows the sequence to line up two of the springs 5302 for insertion into the clip slots and FIG. 56B shows a continuation of the process where the springs are inserted and the grille 1710 is pulled against bottom surface of the UV air treatment body 1702 due to forces from the retention springs. With the gasket discussed previously, this process can seal the grille 1710 to the UV air treatment body preventing airflow from the plenum directly to the UV air treatment body inlet and also providing a clean finished interface between the grille 1710 and the UV air treatment body without any gap, perhaps as best shown in FIG. 65.

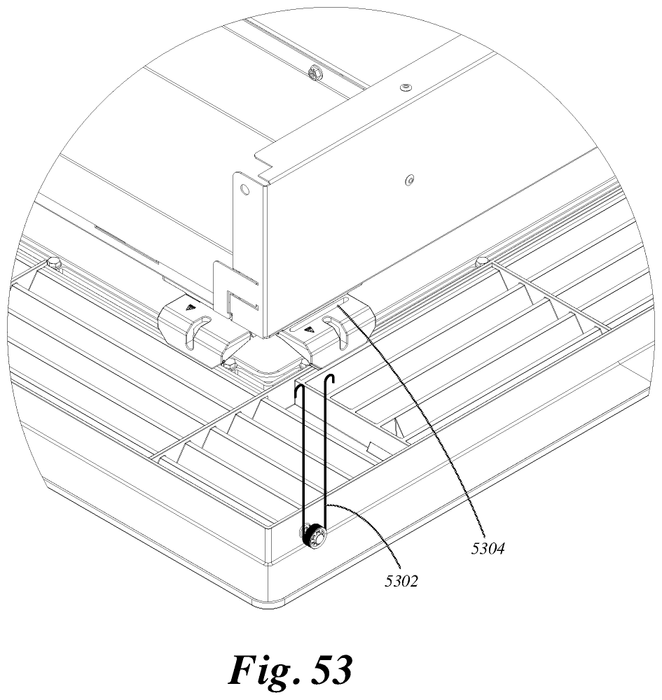

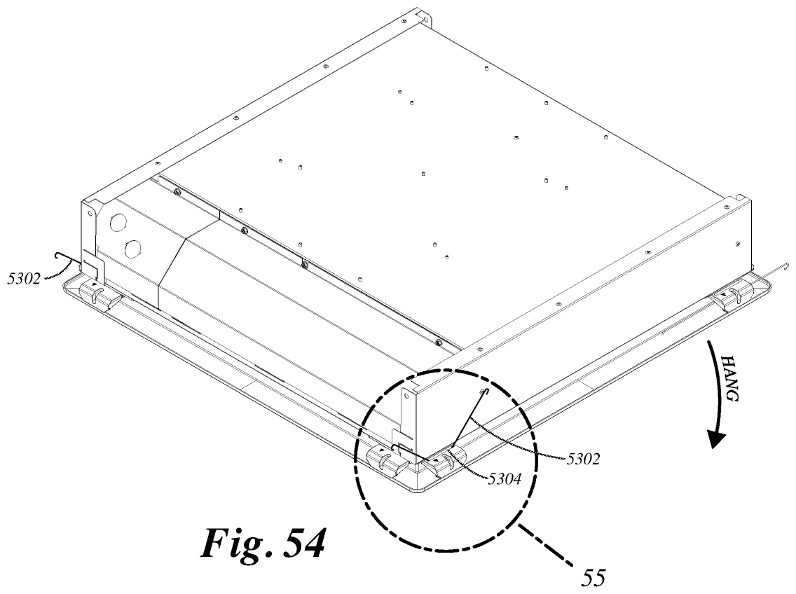

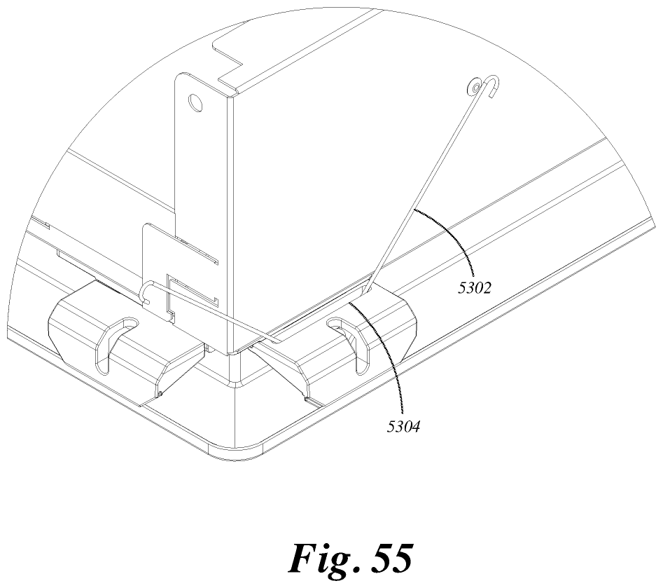

The installation can be done in parts to ease the process and also aid with any supplemental installation (e.g., electrical installation between the grille and body). To hang the grille 1710 in an installation open state, first begin with aligning two of the retention springs 5302 on opposite sides of the grille into their respective clip slots 5304 (See FIGS. 52-53). FIG. 52 illustrates two of the retention springs 5302 being aligned for insertion into their respective slots while the other retention springs 5302 are left unaligned, to be installed later once the grille is ready to be positioned into its installed closed state. Inserting just those two springs 5302 into their respective will allow the grille to hang in an installed open state from the UV air treatment body without aid from the installer (See FIGS. 54-55).

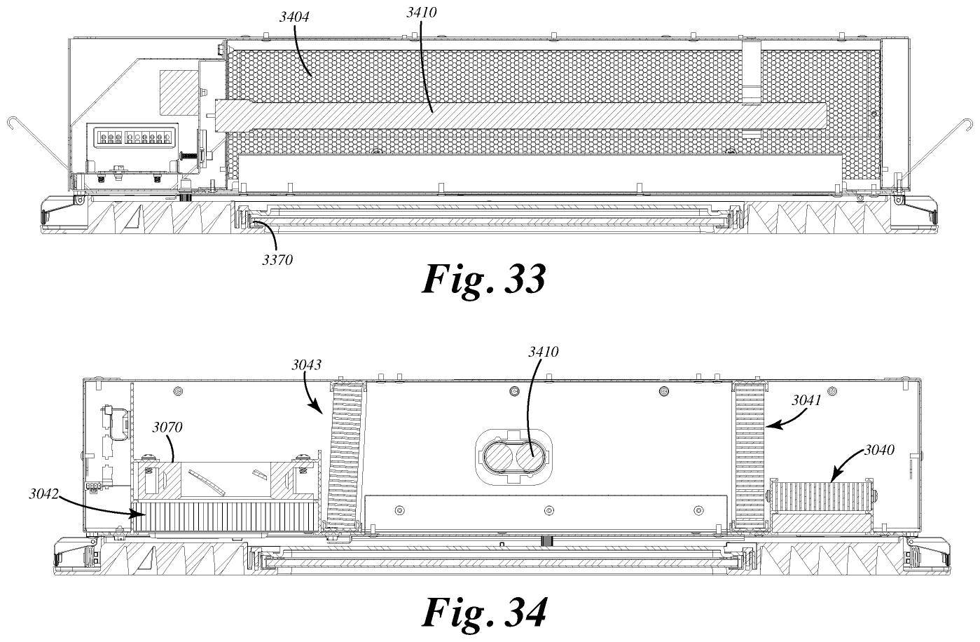

With the grille hanging, any electrical wiring between the grille 1710 and the UV air treatment body 1702 can be performed by the installer. For example, if the grille has a downlight, downlight wires can be routed from the grille to the UV air treatment body. In the embodiment depicted in FIGS. 33-34, the LED 3370 has a two-wire grille latching connector that connects to a UV air treatment electrical connection behind the UV air treatment connection cover 490 on the bottom face of the UV air treatment body. With power connected, the front of the grille can be raised to engage the remaining two springs with their respective clip slots and then the entire grille can be lifted to make contact with the t-grid.

With the UV air treatment fixture fully installed, power for the unit can be connected to the electrical connectors 492. The connectors can include electrical connectors for fixture power and downlight power (or other functional modules in the grille). This process can include connecting WAGO input power receptacle(s) with fixture plug(s). In the current embodiment, power can be provided unswitched to the lamp ballast and switched to the LED downlight driver. A knockout plate or other type of cover can be utilized to hide and protect the electrical connections.

The UV lamp 412 in the current embodiment is rated for about 9000 hours of continuous use. The grille can be easily repositioned to an open state so that the UV lamp 412 and the filter 420 can be replaced. This procedure can include disconnecting power from the fixture, lowering the grille and detaching front springs from the fixture vertical installation clip slots allowing it to hang from the back springs, loosening the chamber access door 428 screw to open the chamber, lowering the access door and allowing it to hang from the fixture. From here, the UV lamp can be pulled from the socket and removed from the retaining clip, and replaced using the same procedure for installation discussed above. The filter 420 can be removed and disposed of according to facility policies and a new filter can be installed in the reverse order of which it was removed. A long press on the filter reset button 496 can be performed to reset the filter and lamp counter prior to raising the grille. A status light can be provided to confirm the unit is powered and fully operational.

### Uniform Airflow Through UV Reactor Chamber

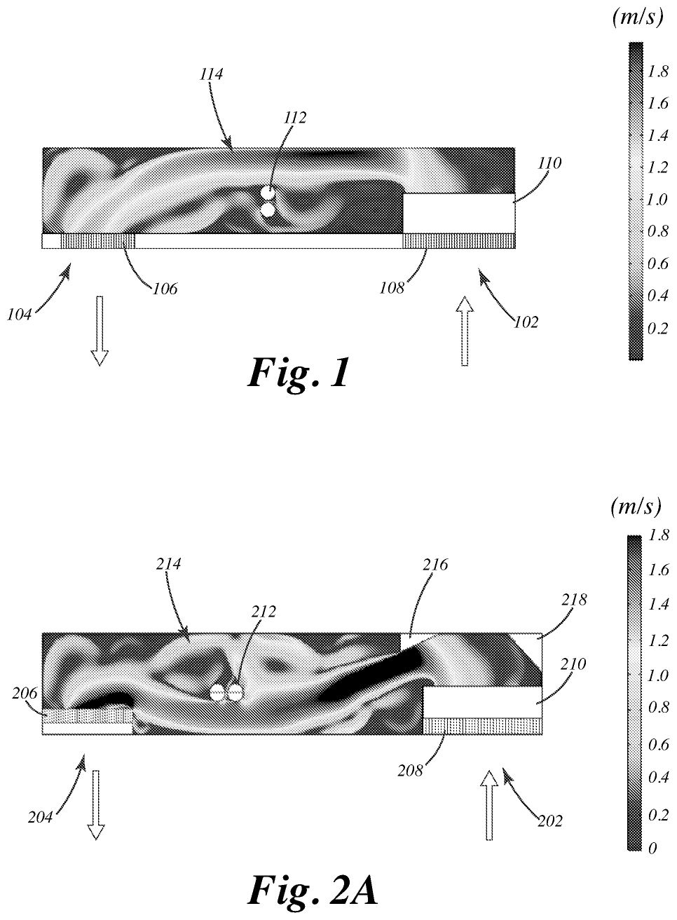

FIGS. 1-3 and 5 illustrate representational airflow diagrams of several embodiments of different UV air treatment fixtures in accordance with the present disclosure. Each UV air treatment ceiling fixture includes an air inlet 102, 202, 302, 502 inlet-side UV reducer air directors 108, 208, 308, 508 a fan 110, 210, 310, 510 a UV reactor chamber 114, 214, 314, 514 a UV source 112, 212, 312, 512 outlet-side UV air directors 106, 206, 306, 506 and an air outlet 104, 204, 304, 504. In general, air is routed through the air inlet 102, 202, 302, 402, 502 through the inlet-side UV reducer air directors 108, 208, 308, 508 to the UV reactor chamber 114, 214, 314, 514 for treatment and then routed through the outlet-side UV reducer air directors 106, 206, 306, 506 to exit the fixture via the air outlet 104, 204, 304, 504. The UV source 112, 212, 312, 512 emits UV light within the UV reactor chamber to mitigate air pathogens in the air pathing through the UV reactor chamber. The UV reactor chamber walls can be generally substantially UV reflective to facilitate providing a suitable UV dosage to the air passing through the UV reactor chamber. The UV reducer air directors allow airflow into and out of the UV reactor chamber, while being configured to cooperatively prevent UV light from escaping the fixture. The direction of the airflow and placement of the various components, such as filters, fans, and the like can vary in different embodiments.

In general, the exemplary embodiments depicted in FIGS. 1-3 and 5 reduce variances in the airflow and increase fluence. These embodiments address the challenge of reducing the size (i.e., the volume of the UV reactor chamber) of a UV air treatment unit while still increasing or providing equivalent fluence. A smaller reactor generally delivers less UV energy unless the air velocity is decreased or other changes are made. Decreasing air velocity, however, generally comes with the tradeoff of treating less of the air in the room. In past UV air treatment designs UV baffles could be bulky taking a large amount of space to prevent UV energy from coming out of the air-flow path and hampering practical installation. The UV reducer airflow directors of the present disclosure (e.g., the honeycomb air flow material) reduce or eliminate UV light from escaping the vents of the unit without the bulk of large UV baffles. As discussed later, the UV reducer airflow directors can be configured to provide light reduction by hole size, depth, and other parameters to provide a desired minimum of UV penetration. This allows the size of the baffles to be decreased (or eliminated) and the size of the reactor to be increased while providing increased airflow and reducing the UV energy allowed to escape.

Air treatment ceiling systems in accordance with embodiments of the present disclosure can enable significant reductions of airborne microbes in a UV air treatment reactor chamber with UV energy. This can be achieved in essentially any environment, including healthcare environments. The reduction in microbes can be accompanied by an associated reduction of risk of acquisition of airborne nosocomial infections.

Some embodiments of an air treatment ceiling fixture in accordance with the present disclosure include a MERV 6 filter (e.g., as shown in FIG. 4A filter 420) along with a 19 Watt ultraviolet (UV) radiation lamp 112, 212, 312, 512 disposed in the main portion of the UV air treatment reactor chamber 114, 214, 314, 514. Other embodiments include a different type of filter, multiple filters, or no filter. Some embodiments include a UV lamp with a different wattage, multiple UV lamps, or another ultraviolet energy delivery system, such as one or more UV light emitting diodes (UV LEDs).

With an airflow of about 50 cubic feet per minute (cfm), one embodiment of the air treatment ceiling fixture can provide an exposure time of about 0.35 seconds and produce a UV dose of about 100 J/m². The recirculation of room air through the air treatment ceiling system assures that virtually complete removal of airborne pathogens in a 100 ft² room can be achieved within about 1 hour, provided there is no ongoing contamination. The UV air treatment ceiling systems in accordance with the present disclosure can outperform many other similar units while consuming low energy because it can be configured for both high performance and low power consumption. Further, quiet operation and a basically invisible recessed profile make the unit suitable for application in essentially any indoor environment where airborne infection transmission may be a concern, such as a hospital environment. It can also reduce contamination of the local environment by removing contact pathogens before they settle out on surfaces.

The ultraviolet energy (dose) delivered to the air in an ultraviolet energy disinfection system generally determines the inactivation rate of microorganisms in the airstream. When modeling the performance of such systems, average dose is widely used and recommended by leading authorities (e.g., Kowalski, Ultraviolet Germicidal Irradiation Handbook, Springer, 2009) in order to estimate a system's inactivation rates. Using the average dose, however, fails to accurately represent the survival rate of microorganisms. Consider a system where half of the air receives infinite dose (inactivating all microorganisms) and the other half of the air receives zero dose. Half of all microorganisms would survive this system (due to half of the air receiving zero dose) despite the fact that the average dose is semi-infinite. Simulations of real air disinfection systems show that using average dose can overestimate microorganism inactivation by a factor of 3 or more.

Instead, microorganism inactivation can be better estimated by determining the percentage of the air that reaches a target dose threshold. In the previous scenario, such an approach would correctly determine that 50% of the air received sufficient dose, and therefore 50% of microorganisms were inactivated. Applying this methodology has shown that for a given system, higher microorganism inactivation rates are generally achieved with more uniform airflow. Accordingly, various aspects of this disclosure emphasize and balance generation of more uniform airflow in the UV reactor chamber to increase microorganism inactivation rates for a given UV air treatment fixture, e.g., for a particular UV reactor chamber size and UV source. In general, the airflow representational diagrams (FIGS. 1-3 and 5) show airflow velocity in meters per second using grayscale shading with the provided scale. In general, target airflow velocities generally range from 0.6 meters/second to 1.4 meters per second. Though target airflow velocity can vary depending on the application. Target airflow velocity can also be configured based on the specific size of the UV reactor chamber and other characteristics of the UV air treatment fixture.

FIG. 1 illustrates a representational airflow model and UV reactor chamber configuration. During operation while installed in a drop ceiling, the fan system 110 draws air from the room through the air intake 102 and the intake-side UV reducer airflow directors 108 and directs it toward the UV reactor chamber 114 where the air is treated by UV energy from the UV source 114 before being directed through the outlet-side UV reducer airflow directors 106 to the air outlet 104. Although not depicted in the representational view of FIG. 1, one or more filters can be disposed in line with the air flow to filter the air. The UV reducer airflow director configurations aid in providing a suitably uniform airflow velocity through the UV reactor chamber 114, as depicted by the shading representing the airflow velocity scale throughout the chamber. The UV reducer airflow directors 106, 108 facilitate a reduction in airflow jet streaming through the UV reactor chamber. This unwanted airflow jet streaming can decrease performance due to reduced pathogen exposure time in high velocity paths.

FIGS. 2A-2B illustrate two alternative air treatment reactor configurations that include airflow disrupters 216, 218, 222. These two configurations are generally similar to the configuration of FIG. 1. However, the main difference is that FIGS. 2A-2B have airflow disrupters 216, 218, 222 that also aid in reducing airflow jet streaming through the UV reactor chamber. The FIG. 2A configuration includes two air flow disrupters 216, 218 that disrupt airflow and assist in providing a more uniform airflow through the UV reactor chamber 214. The FIG. 2B configuration includes the two air flow disrupters 216, 218 as well as an additional air flow disrupter 222 toward the middle of the UV reactor chamber. As depicted by the shaded airflow velocity modeling, in both embodiments, the fan system 210 draws air through the inlet 202 and the inlet-side UV directors 208 pushing air through the reactor 214 with a UV lamp 212. The air flow disrupters 216, 218, 222 help to distribute the air flow path throughout the UV reactor chamber and increase airflow velocity uniformity. Once air flows across the UV chamber, it is pushed by the fan 210 through the outlet-side UV reducer airflow director 206, filter 220, and then out the airflow outlet 204. The air flow disrupters can be sized, shaped, and positioned to increase uniformity of airflow within the UV reactor chamber. In the FIG. 2A embodiment, the airflow disrupters 216, 218 are physical notches that jut inward from the edge of the UV reactor walls. They are generally triangular or ramp shaped. The shape, size, and position of the airflow disrupters can be selected to provide a target airflow velocity within the chamber based on simulation or experimentation of airflow velocities for specific UV reactor geometries. Additional, fewer, or different airflow disrupters can be added to change the airflow patterns in the UV reactor chamber. For example, in FIG. 2B, the additional airflow disrupter 222 can be positioned toward the center of the UV reactor chamber 214 to disrupt an area of high velocity air flow. In general, the air flow disrupters 216, 218, 222 can be selected and configured to cooperate to disrupt airflow jet streaming, e.g., by disrupting higher airflow velocity areas and directing airflow to lower velocity areas of the UV reactor chamber.

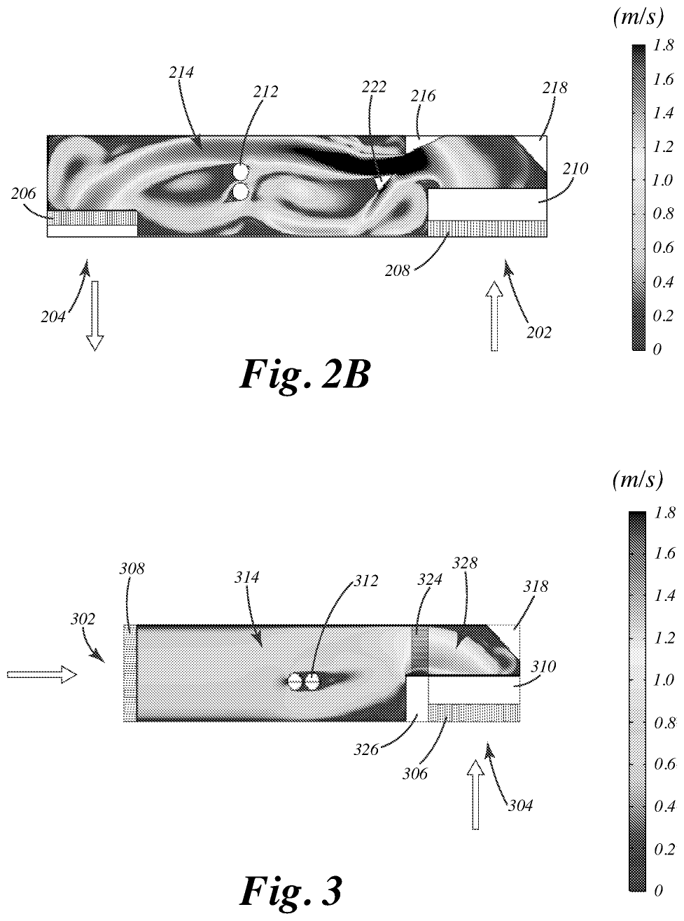

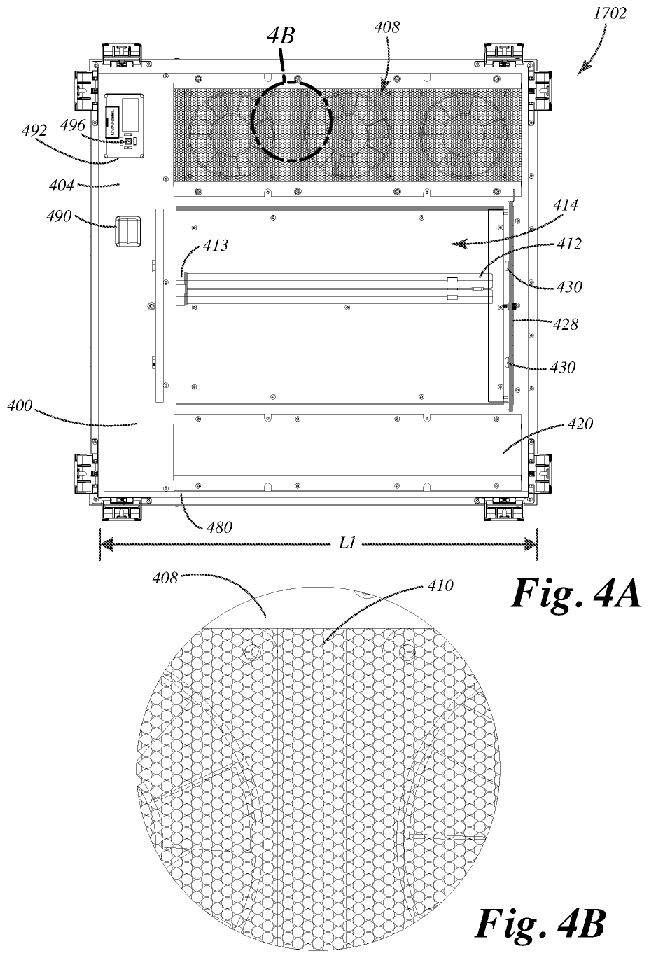

FIG. 3 illustrates a portion of an alternative air treatment reactor configuration. In this embodiment, a UV reducer airflow director 324 is positioned in an air passage between an auxiliary portion of the UV reactor chamber 328 and a main portion of the UV reactor chamber 314. This configuration has some similarity to the previous configurations—the fan system 310 draws air through the inlet 302 and the inlet-side UV directors 308 pulling air through the reactor 314, 328. In this embodiment there is an airflow disrupter 318 in the auxiliary portion of the UV reactor chamber 328 that facilitates redirecting the airflow through the UV reducer airflow director 324. In this configuration, the UV source 312 is positioned toward the exhaust side of the UV reactor chamber and further facilitates disruption of the airflow. At the opposite end of the main portion of the UV reactor chamber 314, the UV reducer airflow directors 306 provide an exit path for the treated air. The air can be directed to outlet 304, which can be positioned adjacent the outlet-side airflow director 306 or toward an additional auxiliary UV reactor chamber (not shown in FIG. 3) for redirecting toward an outlet in the bottom of the UV air treatment fixture. This embodiment provides high air treatment performance due to the increased uniformity of the air velocity within the chamber, as shown by the air flow velocity model. In the FIG. 3 embodiment, the effective fluence is estimated at about 15 mJ/cm².

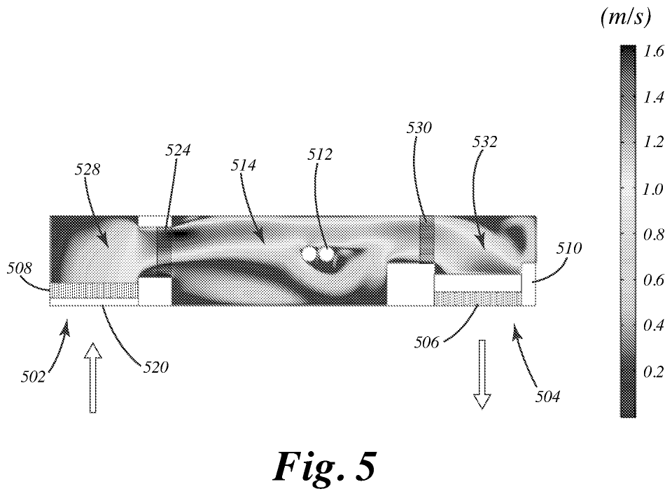

FIG. 5 illustrates another alternative air treatment reactor configuration. In this configuration, the fan 510 is located near the outlet 504 and draws air through the UV reactor chamber 514 from the inlet 502 located on the opposite side of the fixture. That is, during operation, the fan 510 draws air through the filter 520 located at the inlet, through the first inlet-side UV reducer airflow director 508, through the inlet-side auxiliary portion of the UV reactor chamber 528, through the second inlet-side UV reducer airflow director 524, through the main portion of the UV reactor chamber 514, through the first outlet-side UV reducer airflow director 530, the outlet-side auxiliary portion of the UV reactor chamber 532 to the fan 510 where airflow is then directed out the second outlet-side UV reducer airflow director 506 and to the airflow outlet 504.

Each of the UV reducer airflow directors facilitates UV reduction and airflow. The UV reducer airflow directors may be referred to as UV reducing airflow collimators. FIG. 4A shows a bottom view of an exemplary UV air treatment fixture without a configurable maintenance door (also referred to as a grille) installed. Because the grille is not installed, FIG. 4A illustrates the bottom of the UV air treatment body exposing the UV reducer airflow director 408. In FIG. 4C two of the four UV reducer airflow directors (the outlet side vertical UV reducer airflow director 408 and the inlet side horizontal UV reducer airflow director 424). The filter 420 obscures vision of the inlet side vertical UV reducer airflow director (not shown) and the vertical UV reducer airflow director 408 obscures vision of the other outlet side horizontal UV reducer airflow director (not shown).

FIG. 4B shows a zoomed in portion of the vertical outlet side UV reducer airflow director 408 of FIG. 4A. In general, the UV reducer airflow director 408 can generally be provided by a black honeycomb plastic block or another component that simultaneously reduces UV light reflection and directs airflow. For example, another UV stable material. The geometry of the UV reducer airflow director can vary, but in general, each UV reducer airflow director can be an array of hollow cells formed between thin black plastic walls 410. The cells are columnar and can be hexagonal, circular, or some other shape.

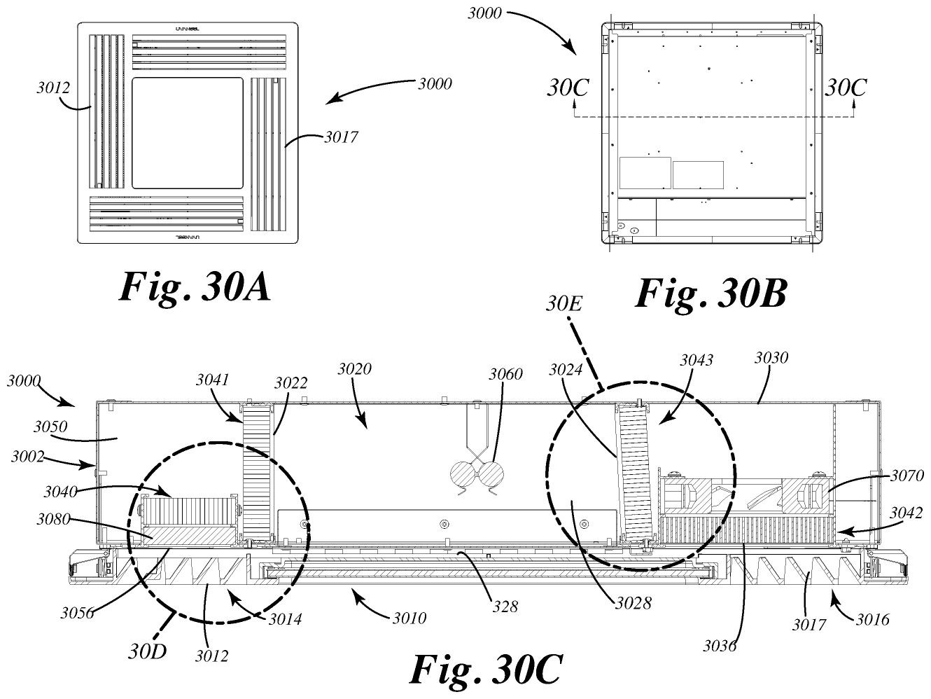

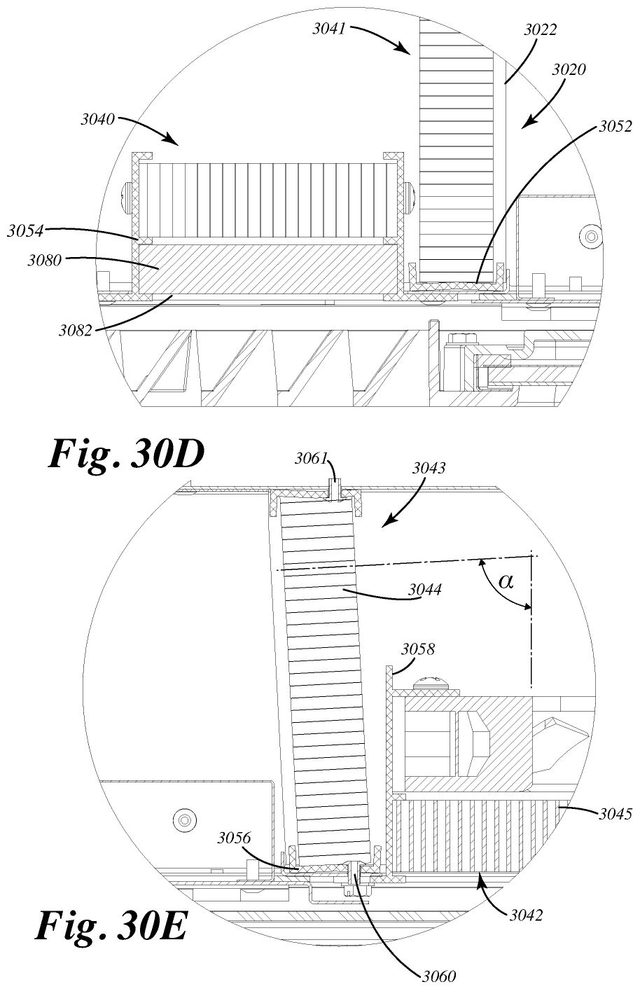

FIGS. 30A-E illustrate an exemplary UV air treatment unit that includes two inlet-side UV reducer airflow directors 3040, 3041 and two outlet-side UV reducer airflow directors 3042, 3043. Airflow during operation of the UV air treatment fixture 3000 is similar to the airflow modeled in FIG. 5. The fan 3070 draws air through a portion of the vent/louver system 3012 through the filter 3080, the first inlet-side UV reducer airflow director 3040, into the UV auxiliary chamber 3050, through the second inlet-side UV reducer airflow director 3041, into the main portion of the UV reactor chamber 3020 via the chamber inlet 3022 where the air is treated by the UV light from UV source 3060, then through the first outlet-side UV reducer airflow director 3043 via the chamber outlet 3024, into the outlet-side auxiliary UV reactor chamber 3030, and then through the second outlet-side UV reducer airflow director 3042 and finally through the outlet vent/louver system 3017.

In FIGS. 1-5 and 30A-E the columnar cells of each UV reducer airflow director generally extend in one direction perpendicular to the face of the UV reducer airflow director. Further, perhaps as best shown in FIG. 30E, multiple UV reducer airflow directors can be installed at an offset angle such that the UV reducer airflow directors cooperate to provide a desired balance of UV light reduction and airflow. By controlling the angle α between two UV reducer airflow directors (e.g., inlet-side 3040, 3041 or outlet-side 3043, 3042), the UV light can be further reduced because the angular relationship between the columnar cell walls 3044, 3045 can enhance the amount of UV light that has to ricochet off the columnar cell walls 3044, 3045 before exiting the UV air treatment body. That is, for example, by orienting the two UV reducer airflow directors such that the angle between them is between about 115 degrees and 75 degrees, UV light from the UV reactor chamber can be reduced significantly as it bounces through the generally horizontally oriented columnar cells of a first UV reducer airflow director into the auxiliary chamber and then bounces through the second set of generally vertically oriented columnar cells of a second UV reducer airflow director. In some aspects, about a 90 degree angle between the horizontal and vertical UV reducer airflow directors may provide satisfactory UV reduction while simultaneously providing satisfactory airflow. In other aspects, an angle less than or more than 90 may provide desired UV reduction and airflow. The angle between the UV reducer airflow directors can be selected to provide a desired UV reduction, desired airflow speed, or a desired balance between the two.

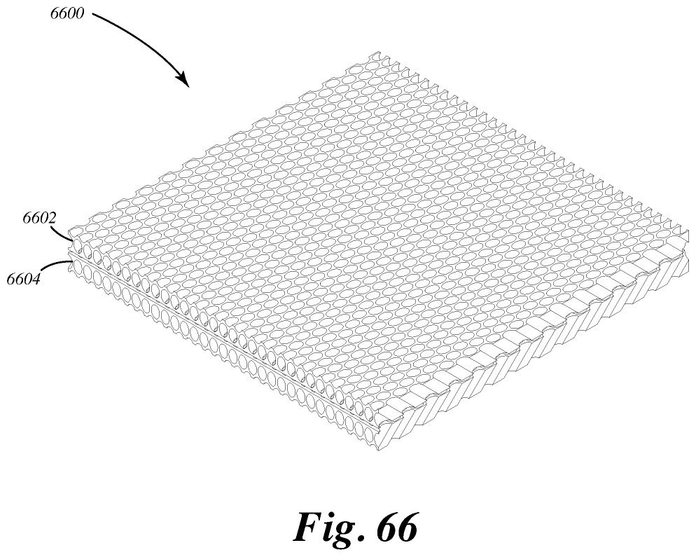

In alternative embodiments, the columnar cells can extend at a different angle relative to the face of the UV reducer airflow director or provide other geometry to provide a more tortured path. FIGS. 66-74 illustrate three exemplary embodiments of UV reducer airflow directors.

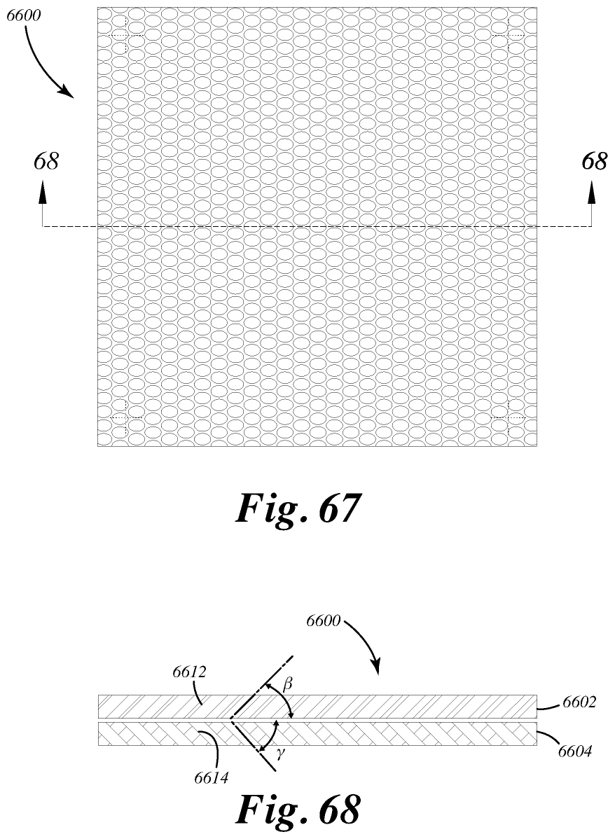

FIGS. 66-68 illustrate a double angled UV reducer airflow director. FIG. 66 illustrates a perspective view of the double angled UV reducer airflow director 6600. The double angled UV reducer airflow director 6600 is formed by aligning and combining two angled UV reducer airflow directors 6602, 6604 together. The two angled UV reducer airflow directors can be combined by fitting them together in a bracket (e.g., such as the U-shaped bracket 3052 of FIG. 30D) or they can be joined together another way, such as by gluing, screwing, taping, or another fastening method. Although FIGS. 66-68 includes two stacked angled UV reducer airflow directors, in alternative embodiments additional angled UV reducer airflow directors can be stacked together to provide a more tortured path for the UV light to travel. Further, in another alternative embodiment, a single angled UV reducer airflow director (e.g., 6602 or 6604 alone) can be utilized alone. FIG. 67 illustrates a top view of the double angled UV reducer airflow director 6600 and FIG. 68 illustrates a sectional view through cut line 68. The two separate angled UV reducer airflow directors 6602, 6604 are shown with a slight separation to emphasize that they can be two separate pieces that can be disposed adjacent to each other (or joined together) to form the double angled UV reducer airflow director 6600. The angles β, γ of the columnar cell walls 6612, 6614 (with respect to the faces of the UV reducer airflow directors) are both about 45 and −45 degrees respectively in the depicted embodiment. In alternative embodiments, the angles can be smaller or larger and may or may not be the complimentary. In some embodiments, the relative angles of the angled UV reducer airflow directors 6602, 6604 can be selected such that a UV light ray cannot pass directly through the double angled UV reducer airflow director 6600 without first bouncing on at least one of the columnar cellular walls 6612, 6614, which causes some of the UV energy to be absorbed.

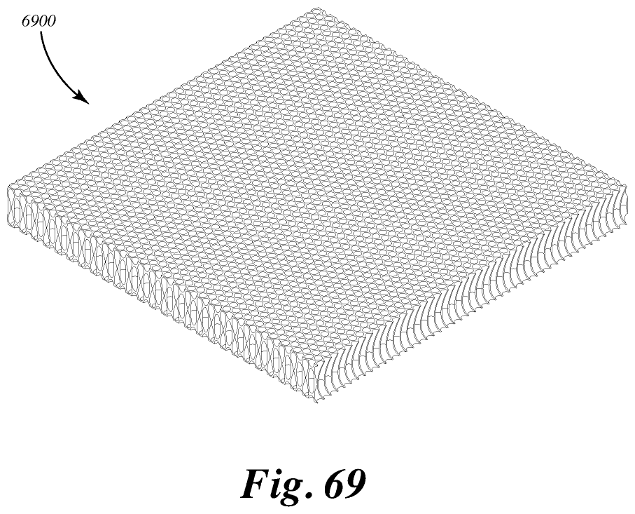

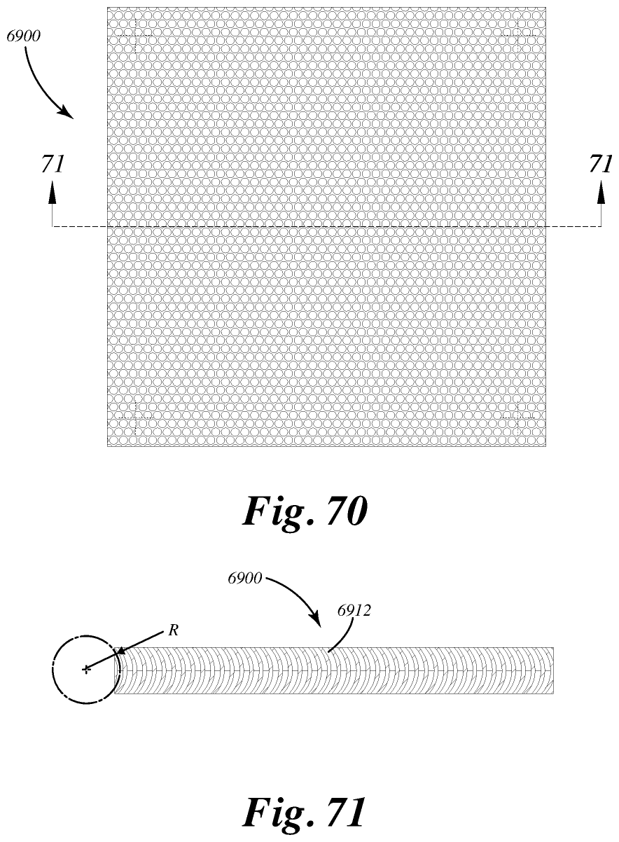

FIGS. 69-71 illustrate a curved UV reducer airflow director 6900. FIG. 69 illustrates a perspective view, FIG. 70 a top view, and FIG. 71 a sectional view cut along line 71 of FIG. 70. The cell walls 6912 of the curved UV reducer airflow director 6900 can have essentially any amount of curvature depending on the selected radius of curvature. In the depicted embodiment, the radius of curvature r is about half an inch. The current embodiment includes a single radius of curvature, but in alternative embodiments a more tortured path can be created for the cell walls by providing multiple bends in the path. Further the bends can be provided at varying curvature. Similar to the double angled UV reducer airflow director 6600, a double curved (or more) UV reducer airflow director can be created by joining (by alignment or fastening) multiple curved UV reducer airflow directors together. For example, an S-shaped path can be formed by joining the current curved UV reducer airflow director 6900 with an identical director that is rotated 180 degrees.

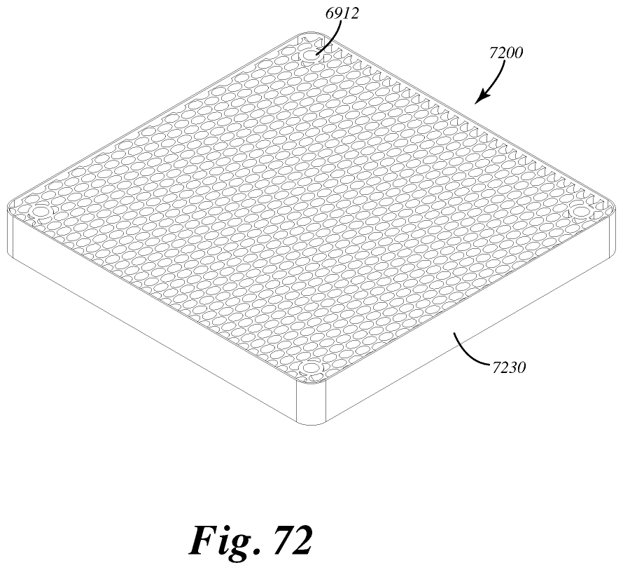

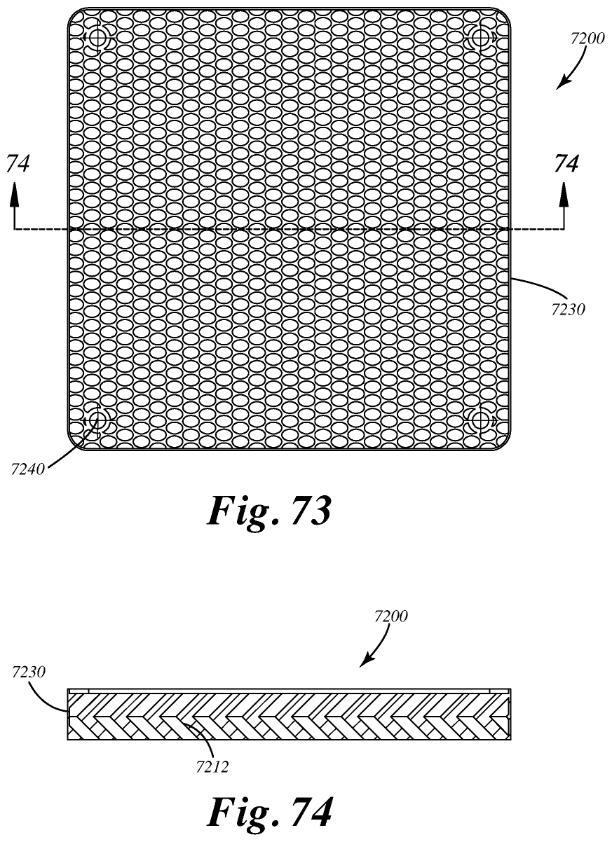

FIGS. 72-74 illustrate a v-shaped UV reducer airflow director 7200. FIG. 72 illustrates a perspective view, FIG. 73 a top view, and FIG. 74 a sectional view cut along line 74 of FIG. 73. The cell walls 7212 of the v-shaped director 7200 provide a similar tortured path as the double angled director 6600 of FIG. 66. The angle of the v-shaped walls can vary depending on the application. The director 7200 of this embodiment includes a director wall 7230 surrounding the outer edge of the director. Further, the director 7200 includes fastening apertures 7240 that can be used to fasten multiple directors together and/or to secure the director in place within a UV air treatment system. Although not depicted in the other embodiments of FIGS. 66-71, similar fastening apertures can be provided in those or other embodiments. Further, angled, v-shaped, and curved directors can be combined to form assorted different tortured paths depending on the application, desired UV reduction and airflow characteristics.

The relative position and orientation of the UV reducer airflow directors can assist in providing target airflow velocities while preventing UV light from escaping the UV air treatment fixture. While the directors can be joined or aligned adjacent to create a tortured path, the directors can also be oriented offset with an auxiliary chamber in-between as shown in FIG. 30A-E. Referring to FIG. 30D, a close-up of the inlet-side UV reducer airflow director positioning is shown. In particular the two inlet-side UV reducer airflow directors 3040, 3041 are positioned and fixed in place with brackets 3052, 3054 such that the two inlet-side UV reducer airflow directors are generally perpendicular to each other. The inlet-side bracket 3052 can include a first ledge for holding the filter 3080 in place and a second ledge for holding the director 3040 in place. Referring to FIG. 30E, a close-up of the outlet-side UV reducer airflow director positioning is shown. In particular the two outlet-side UV reducer airflow directors 3042, 3043 are positioned and fixed in place with brackets 3056, 3058 such that the two outlet-side UV reducer airflow directors are generally oriented at angle α apart (about 80 degrees in the current embodiment). The outlet-side bracket 3058 can include a first ledge for holding the fan assembly 3070 in place and a second ledge for holding the director 3042 in place. The other outlet-side bracket 3056 can be configured to provide the relative angle α between the UV reducer airflow directors 3042,3043. That is, such that the angle α between the columnar cell walls 3044 of director 3043 and the columnar cell walls 3045 of director 3042 is at about 80 degrees. In the current embodiment, the configuration can be provided by angling rivets 3060,3061 that impinge respectively on the top and bottom edge of the director 3043 to hold it at a fixed orientation relative to the bracket. In alternative embodiments, the fixed orientation of the bracket can be provided in a different manner. For example, in one alternative embodiment illustrated in FIG. 37, a single bracket 3055 can be utilized for holding two directors at a specific relative orientation.

The configuration of FIG. 30A-E prevents UV light from taking a direct path out of the reactor 3020 and into the room below the fixture. During operation, UV light reflects all around the UV chamber 3020 due to the reflective surfaces lining the chamber. Some UV light will be directed toward the UV reducer airflow directors 3041, 3043 that form part of the chamber walls. The UV light will arrive at the UV reducer airflow directors at various different angles. Due to the columnar and cellular structure of the UV reducer airflow director UV light will be incident with the cell walls and because of the UV reducing properties of the black plastic, much of the UV light incident with the cell walls of the UV reducer airflow director will be absorbed. To the extent that some UV light reflects off of the UV reducer airflow director or some UV light rays are not incident with the cell walls of the UV reducer airflow director, some UV light can reach the auxiliary chamber 3050. Such UV light has still not escaped the UV air treatment fixture. The UV light may be absorbed by the auxiliary portion of the UV reactor chamber walls. Some UV light may be reflected toward the other inlet-side UV reducer airflow director 3040 that is arranged perpendicularly to the inlet-side UV reducer airflow director 3041. Accordingly, UV light incident with the UV reducer airflow director 3040 cell walls will be further absorbed. And, to the extent that UV light reflects or passes through the second UV reducer airflow director 3040, the intensity of the UV light is below target threshold levels. Essentially, the positioning and orientation of the UV reducer airflow directors 3041, 3041 cooperate such that UV light is either absorbed or has a sufficient number of reflections to lower the intensity to suitable levels. The UV light reflection and transmission through the UV reducer airflow directors can vary depending on a number of different characteristics. For example, the diameter-to-length ratio of the UV reducer airflow directors can vary the UV light reflected and transmitted through the UV reducer. For a specific UV reducer airflow director, the relationship between cell hole diameter D, length L, and the number of reflections n, is given by the following formula:

$$\frac{D}{L} < \frac{1}{\frac{n}{2} + 1}$$

Based on the reflectivity of the UV reducer airflow director material and how much energy is lost at each reflection, a number of reflections can be determined for a particular UV reducer airflow director. The diameter-to-length ratio for each UV reducer airflow director can be selected based on the number of reflections given its specific characteristics and the desired UV reducing properties. For example, in the FIG. 30A-E embodiment, each UV reducer airflow director has a minimum reflection ratio of about 3.5 to 1. When two UV reducer airflow directors are used in tandem with an offset angle, such as being disposed perpendicularly, the minimum number of reflections of UV light from the UV chamber to the outlet can ensure that any UV light escaping the UV air treatment fixture is below a predefined intensity level. The minimum intensity level can be adjusted by changing the number, arrangement, and characteristics

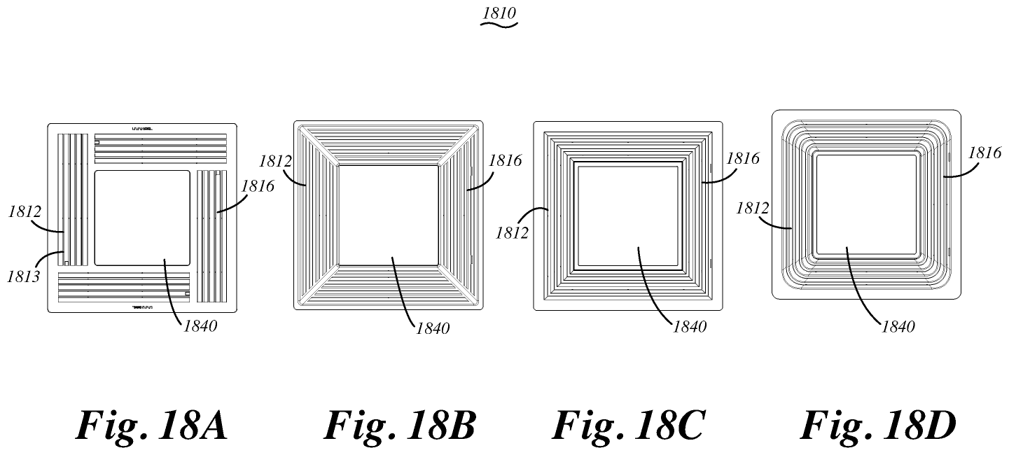

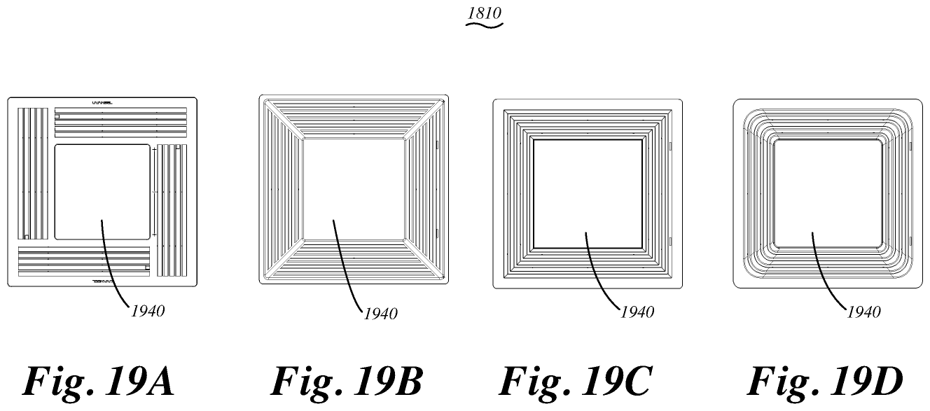

The outlet 3016 may include at least one outlet louver 3017 defining the outlet opening 3018. The outlet louver 3017 may alternately be referred to as an outlet vent. The outlet louver 3017 can be configured to direct the airflow out of the outlet chamber 3030. The at least one outlet louver 3017 may have any suitable louver orientation, such as those described in connection with FIGS. 18A-D and FIGS. 19A-D. In one embodiment, the outlet louver 3017 can have the same louver orientation as the inlet louver 3013. In another embodiment, the outlet louver 3017 can have a different louver orientation from the inlet louver 3013. The inlet louver 3013 and the outlet louver 3017 may be configured to absorb UV light emitted from the germicidal light source 3060. Put another way, the inlet louver 3013 and the outlet louver 3016 may interact positively with the UV light. The inlet louver 3013 and the outlet louver 3016 can provide one or more additional reflection for the UV light emitted from the germicidal light source 3060 to reduce the amount of UV light escaping the air treatment ceiling system 3000.

The inlet louver 3012 and the outlet louver 3017 provide a pathway for air into and out of the air treatment ceiling system 3000 respectively through a configurable maintenance door 3010, which will be discussed in more detail below. The inlet louver 3012 and the outlet louver 3017 can be configured to reduce the amount of UV light escaping the air treatment ceiling fixture 3000 without unduly restricting airflow into and out of the air treatment ceiling fixture 3000. The inlet louver 3012 may be configured at an angle relative to the inlet chamber opening 3056 and the outlet louver 3017 may be configured at an angle relative to the outlet chamber opening 3036. As depicted in FIG. 30C, the outlet louver 3017 can be configured at an angle relative to the collimator 3042 at the outlet chamber opening 3036. As depicted in FIGS. 30C and 30D, the inlet louver 3012 may be configured to align with the inlet chamber opening 3056 through the air filter 3080 but at an angle relative to the UV reducer airflow director 3040 nearest the inlet chamber opening 3056. In an alternate embodiment, the inlet louver 3013 can be configured at an angle relative to the air path through the air filter 3080.

Table 1, below, provides exemplary UV light reduction results for the outlet of a UV air treatment unit. Two sets of results are provided for two different cell hole sizes (1 inch and ¾ inch) at a ⅛ inch depth. For these examples, the UV reactor has a 60 Watt UV source. In essence, the characteristics and configuration of multiple UV reducer airflow directors can be selected to reduce UV energy from the reactor to the area outside the UV air treatment fixture such that suitable airflow through the reactor for satisfactory dosing is providing but no UV penetration above a particular intensity level is allowed to escape the unit. Although the table provided shows the reduction in UV intensity at the outlet, similar reductions are provided at the inlet with the same two perpendicular UV reducer airflow director configuration.

TABLE 1

| UV chamber | | | | |
|---|---|---|---|---|
| Outlet-side UV | Outlet UV | UV | | |
| reducer airflow | reducer airflow | Intensity | | |
| director | director | (uW) | % Reduction | Lux |
| ⅛″ × 1″ | ⅛″ × 1″ | 0.0015 | 99.9% | No |
| Black Plastic | Black Plastic | | | reading |
| Honeycomb | Honeycomb | | | |
| ⅛″ × ¾″ | ⅛″ × ¾″ | 0.015 | 99.4% | No |
| Black Plastic | Black Plastic | | | reading |
| Honeycomb | Honeycomb | | | |

The UV air treatment systems of the present disclosure include a control system for dynamic air pathogen mitigation. The control system can be configured to control operation of the UV air treatment system in order to influence and mitigate critical biological or exposure conditions.

The system can track and manage pathogen levels to provide an improved understanding of UV air treatment impact and dynamic control within environments. The UV air treatment system can assist in defining and enabling healthier environments by integration into a larger automated health management system. Further, by collecting relevant sensor data while dynamically mitigating pathogens within an environment, performance metrics can be tracked and assist in enabling new automated systems and methods.

The UV air treatment fixtures of the present disclosure contain the UV-C source irradiance within the unit to meet applicable standards. Current U.S. standards general limit UV-C leakage measured at any point on the surface of the unit to about 0.1 uW/cm2.

For UV air treatment fixtures in accordance with the present disclosure there is a balance to be struck between air flow velocity, UV-C leakage, and reactor size. Put simply, in order to provide an effective dosage of UV energy in a smaller UV reactor chamber footprint (e.g., 2×2 instead of 2×4), the present disclosure balances the reduced size with improvements for absorbing/redirecting the UV light while maintaining satisfactory airflow velocities. Further, the embodiments of the present disclosure provide these solutions without generating noticeable noise in the room.

More detail will now be provided about various embodiments of UV reducer airflow directors. As mentioned above, in general a UV reducer airflow director can be an array of columnar cells. The array of columnar cells can be provided by a set of black plastic honeycomb ⅛″ diameter tubes with a depth of about ¾″. Two sets of the tubes can be arranged in a perpendicular orientation to block nearly all of the UV-C and visible light spectrum from a UV Source. The material for the array of columnar cells need not be limited to black plastic nor the particular diameter and depths. The characteristics of the UV reducer airflow directors can vary depending on the application and various characteristics of the UV air treatment system including. Various embodiments of UV reducer airflow directors can include black painted aluminum, bare aluminum, and black plastic tubes at varying depths and diameters. In general, there is a tradeoff between UV reactor volume and light reduction, but the UV reducer airflow director that offers a reasonable tradeoff for a UV reactor that fits within a 2×2 unit ceiling cell is a ⅛″×¾″ black plastic honeycomb UV reducer airflow director. A hole diameter to UV director depth ratio of 6 to 1 generally provides a satisfactory balance between airflow and UV light reflection for a 2×2 foot ceiling tile UV air treatment fixture installation.

UV reducer airflow directors do not negatively impact noise levels produced by the UV air treatment fixture. Table 2 shows estimated sound levels generated by a UV air treatment system with UV reducer airflow directors. Some embodiments of the present disclosure utilize multiple fans, e.g., three fans, instead of a single fan like some previous UV air treatment fixtures. The use of multiple fans to generate airflow through the unit contributes to improved acoustics over some previous UV air treatment systems because multiple fans can be operated at a lower total a-weighted decibels (dBA) to achieve the same CFM as one fan in some previous UV air treatment system. In addition, individual fan RPM for each fan can be adjusted to provide more or less air flow depending on the desired room noise vs. pathogen mitigation performance.

Noise Performance with ⅛″×¾″ Black Plastic Honeycomb

TABLE 2

| | | Velocity (ft/min) | | Sound (dBA) |
|---|---|---|---|---|
| | | Measured @ | | Measured @ |
| Voltage | Current | the center | Estimated | the center |
| (Volt) | (Amps.) | of outlet | CFM | of unit |
| 6 | 0.42 | 210 | 40.8 | 39.30 |
| 8 | 0.59 | 341 | 66.3 | 44.90 |
| 10 | 0.78 | 385 | 74.9 | 50.50 |
| 12 | 0.98 | 430 | 83.6 | 53.60 |
| 14 | 1.18 | 553 | 107.5 | 57.00 |
| Ambient Noise | | | | 25.00 |

Since modeling of air units began, it has become clear that not all the air flowing through a unit experiences uniform disinfection. Particle tracing simulations for various 2×4 and 2×2 designs have all shown skew-right distributions where the particles receiving the most fluence have 2-5 times more energy than particles receiving the least. That is, in general, previous UV air treatment ceiling fixtures have values of fluence that range from 50-200 mJ/cm2. This raises the question of how to represent the effective fluence of an air unit, i.e. a fluence value that accurately describes our unit's overall removal rate, despite non-uniform treatment. Previous analysis has used the arithmetic mean (average) fluence that particles would receive to predict overall disinfection. However, the arithmetic mean significantly overestimates a unit's disinfection. While the equation for precise disinfection values varies from unit to unit, a rough estimate that is more accurate than the arithmetic mean can be determined, which is the effective fluence at D₉₀=6 mJ/cm2.

Fluence is used synonymously with dosage to mean energy per unit area. When discussing air systems, it generally refers to the energy per unit area incident on the surface of an infinitesimal sphere, which represents the energy a pathogen would be expected to receive.

D₉₀ values are the coefficients unique to each pathogen and each wavelength of UV light that relate fluence to the logarithmic survival rate. Survival rate(s) refers to the percentage of pathogens that survive a given UV exposure.

Effective fluence is the average fluence each particle in a system would have to receive in order see the overall survival rate that is observed in that system. If fluence in a system has a uniform distribution, average fluence and effective fluence will be equal. If the fluence distribution is non-uniform, however, effective fluence and average fluence will be different.

A particle tracing analysis can be performed for UV air treatment units to create an initial distribution of particles that is proportional to velocity at the unit inlet. Each particle represents an approximately equal amount of air that enters the system. The total survival rate for pathogens that travel through an air unit will therefore be equal to the average survival rate associated with each particle. Effective fluence will then be given by:

$${Fl}_{eff} = {D}_{90} * - log 10 ( \frac{\sum {s}_{i}}{{n}_{p}} )$$

Where Fl_{eff} is effective fluence, si is the survival rate associated with particle i, and np is the total number of particles that are analyzed in the simulation. Using a first order approximation of survival rates, the above equation becomes:

$${Fl}_{eff} = {D}_{90} * - log 10 ( \frac{\sum 10 \hat{} ( - \frac{{Fl}_{i}}{{D}_{90}} )}{{n}_{p}} )$$

This method of determining effective fluence introduces two complicating factors. First, instead of calculating average fluence (for which no particle tracing simulations are necessary to create a reasonable prediction) each particle's individual fluence (Fli) must be tracked. Example 1, below, highlights this. Second, the average survival rate is a function of D₉₀ value, which means a unit will have different effective fluence values for different pathogens. While this may seem counterintuitive given that each particle's actual fluence is independent of D₉₀, Example 2 helps illustrate why this would be the case.

Example 1: Mean fluence is inaccurate. A system can be modeled using two particles, each of which represents half of the air entering that system. For the pathogen of interest, a particle would need to receive 50 mJ/cm2 to achieve 100% removal. Particle A receives 100 mJ/cm2, and has a pathogen survival rate of 0%. Particle B receives 0 mJ/cm2, and has a pathogen survival rate of 100%. The overall survival rate is therefore 50%.

If average fluence is used to predict overall disinfection, however, the removal rate will be inflated. The average fluence received by all particles is 50 mJ/cm2, which corresponds to a pathogen survival rate of 0%, not the 50% we know the answer to be.

Example 2: D₉₀ values affect effective fluence. A system can be modeled using two particles, each of which represents half of the air entering that system. Particle A receives 100 mJ/cm2, while Particle B receives 0 mJ/cm2. Pathogen 1 has a D₉₀ value of 50 mJ/cm2, while Pathogen 2 has a D₉₀ value of 100 mJ/cm2. For pathogen 1, Particle A has a survival rate of 1%, and Particle B has a survival rate of 100%, resulting in an average survival rate of 50.5%. This corresponds to a log reduction of 0.297, which, for Pathogen 1, would be an effective fluence of 14.9 mJ/cm2. Doing the same analysis for Pathogen 2, the survival rate of Particle A is 10% and of Particle B is 100%, resulting in an average survival rate of 55%. This corresponds to a log reduction of 0.26, which, for Pathogen 2, would be an effective fluence of 26 mJ/cm2. Thus, even though the total fluence was the same for each particle and each pathogen, the effective fluence varies based on D₉₀.

Since the relationship between D₉₀ and effective fluence is dependent on the distribution of particle fluence, no first principles relationship is expected to exist. Instead, an empirical relationship can be calculated for the two terms. For example, one previous UV air treatment system provided the following effective fluence:

$${Fl}_{eff} = 0.0015 {D}_{90}^{3} - 0.0717 {D}_{90}^{2} + 1.5375 {D}_{90} + 52.86 [ \frac{mJ}{cm 2} ]$$

The r² value for this fit was 0.9995.

While this sort of analysis and precise equation can be obtained for any design that has been simulated, it can be time consuming to perform for each design iteration. A quick estimate of effective fluence that is more accurate than mean fluence can be provided by calculating effective fluence at D₉₀=6 mJ/cm2. Obtaining this value for a given design can be done relatively quickly and will provide an estimate that is in between the effective fluence for both low and high resistance pathogens.

Table 3, below, provides exemplary effective fluence values as a function of D₉₀ for a UV air treatment fixture in accordance with the present disclosure.

TABLE 3

| D₉₀ | | EffectiveFluence |
|---|---|---|
| (mJ/cm2) | LogReduction | (mJ/cm2) |
| 1 | 54.1 | 54.1 |
| 2 | 27.9 | 55.8 |
| 3 | 19 | 57 |
| 4 | 14.5 | 58 |
| 5 | 11.8 | 59 |
| 6 | 9.98 | 59.88 |
| 7 | 8.66 | 60.62 |
| 8 | 7.66 | 61.28 |
| 9 | 6.88 | 61.92 |
| 10 | 6.25 | 62.5 |
| 11 | 5.734 | 63.074 |
| 12 | 5.3 | 63.6 |
| 13 | 4.93 | 64.09 |
| 14 | 4.61 | 64.54 |
| 15 | 4.33 | 64.95 |
| 16 | 4.09 | 65.44 |
| 17 | 3.87 | 65.79 |
| 18 | 3.68 | 66.24 |
| 19 | 3.51 | 66.69 |
| 20 | 3.35 | 67 |

The UV reactor chamber includes a reflective material such that the UV light from the UV source reflects off the chamber walls. Various different reflective materials can be utilized in accordance with embodiments of the present disclosure. Whether the reflective material is diffuse or specular generally affects the effective fluence. Further, the reflectivity of the particular material can also influence the fluence. For example, embodiments of the present disclosure can utilize a material with a reflectivity of 0.9.

Diffuse and specular reflector material are both viable. There may be minor advantages for one or the other based on the geometry of the reactor or other factors. Some embodiments utilize diffuse reflectors for a 2×2 unit footprint because available diffuse materials have better reflectivity at their price point, though prices can fluctuate.

Uniform Air Flow. Higher effective fluence can be achieved with more uniform airflow through the UV air treatment unit. This can be demonstrated from the equations governing effective fluence and can also be confirmed by modeling test cases.

In some UV air units, fans are located at the inlet and are oriented and configured to blow air through the unit. In general, as fans blow air into a UV reactor chamber, a jet forms at the inlet that reduces effective fluence because of the non-uniformity it introduces. Reversing the direction of airflow so that the fans are located at the outlet and draw air through the unit substantially reduced the jet streaming and resulted in a substantial increase in the uniformity of airflow through the unit.

FIGS. 1, 2A, and 2B illustrate exemplary embodiments where the fans are located at the inlet and push air through the unit, while FIG. 5 illustrates an embodiment where the fan is located at the outlet and draws air through the unit from the inlet due to the configuration of the fans. In this type of configuration, airflow is much more uniform. Effective fluence was ˜15 mJ/cm2, nearly double the effective fluence using the same size reactor and bulb.

Increasing dosage and performance in a smaller air treatment unit. Embodiments of the present disclosure can provide equivalent or enhanced fluence in a smaller footprint UV air treatment system. For example, equivalent or enhanced treatment performance can be provided with a UV reactor chamber of a 2×2 foot unit over a 2×4 footprint by incorporating one or more aspects of the present disclosure. One contributing factor are UV reducer airflow directors. A UV reducer airflow director can be a honeycomb molded air guide that acts as a light baffle while permitting satisfactory airflow through the system. The UV reducer airflow director compresses the overall light leakage solution providing better dosage in a smaller package by adding air volume to the reactor. Reverse air flow is another aspect that can be incorporated to improve air velocity distribution. Specifically, by pulling or drawing air through the system instead of pushing air through the system a more even airflow for better velocities and dosage can be provided. Another factor to providing effective performance in a smaller footprint is the use of diffuse reflective material. In particular, the UV reactor chamber can utilize a highly diffuse and reflective Polytetrafluoroethylene (PTFE) coating or layer on the internal surface areas of the reactor chamber to help improve reactor efficiency for higher dosage in a smaller design. Yet another factor to providing effective performance is lamp position. The UV lamp can be positioned within the reactor for efficiency. Different placements within the reactor can reduce turbulence and lower velocity changes for better dosage in a smaller package. The position of the UV lamp can be selected to increase uniformity of airflow through the chamber at operating airflow velocities.

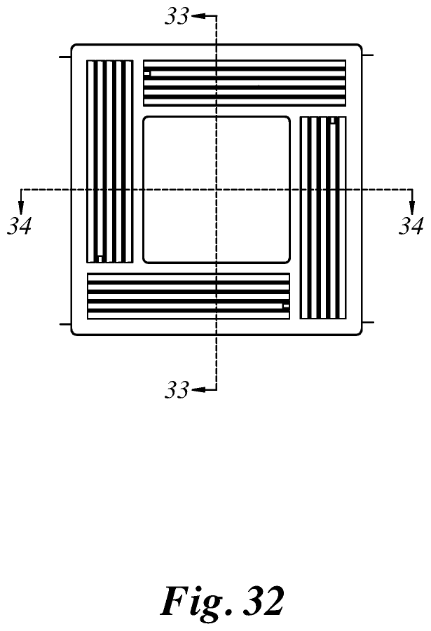

FIGS. 32-34 illustrate one embodiment of a UV air treatment unit or fixture. FIG. 32 illustrates a bottom view having an air inlet, air outlet, and a visible downlight. FIG. 33 illustrates a sectional view along line 33 of FIG. 32 and FIG. 34 illustrates a different sectional view along line 34 of FIG. 33. The UV reducer airflow directors and representative airflow is perhaps best seen in FIGS. 33-34. The figures may not be to scale in order to assist with providing visual clarity about the placement and details of the UV reducer airflow directors and other components.

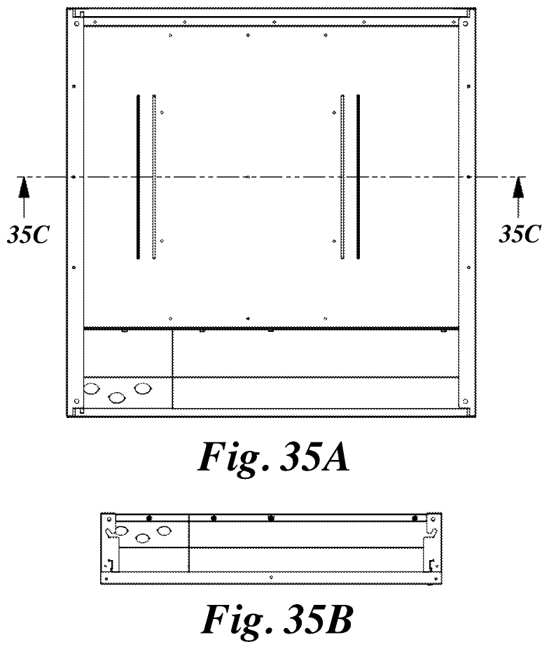

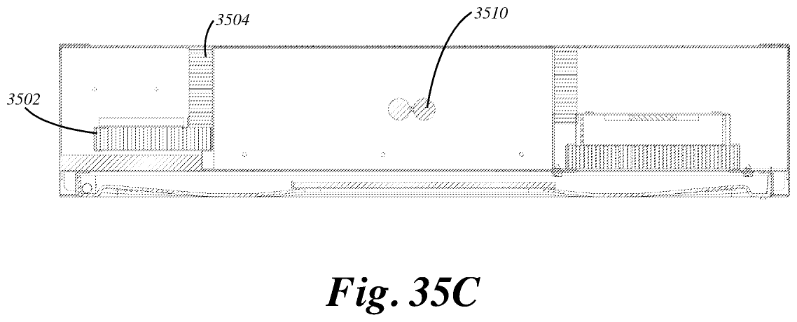

FIGS. 35A-C illustrate another embodiment of a UV air treatment unit or fixture. FIG. 35A illustrates a top view of the unit, FIG. 35B illustrates a side view. FIG. 35C illustrates a sectional view along line AI-AI of FIG. 35A.

The UV reducer airflow directors 3502, 3504, 3506, 3508 of this embodiment can perhaps best be seen in FIG. 35C. The exemplary UV reducer airflow directors depicted are a black honeycomb plastic material that simultaneously reduces UV light reflection from the UV source 3510 in the reactor and directs airflow. As depicted the UV reducer airflow directors are each an array of hollow cells formed between thin black plastic walls. The cells are columnar and have circular holes. In this embodiment two of the UV reducer airflow directors are held in a specific relative orientation by the holder 3055 depicted in FIG. 37.

### Control System

FIG. 24 shows an exemplary disinfection control system 2400 for a UV air treatment ceiling fixture in accordance with the present disclosure. As described herein, a UV air treatment ceiling fixture or unit in one embodiment may include a control system 2400 configured to control operation of the UV air treatment ceiling unit and components thereof. A control system 2400 in accordance with one embodiment is shown in FIG. 24. In one embodiment, the control system 2400 may be configured as an Internet of Things (“IoT”) hub or node within a network, as described herein.

The control system 2400 may include power management capabilities and an optional battery management system for safety and emergency purposes. One or more sensors may be provided to detect in room conditions for general data usage and analytics as well as helping to inform the systems control of events and conditions for response. The system may include an industrial automation interface for control and energy management. The control system may include a UVC sensor to understand dose and time for the air reactor and the surface treatment. Power management may include one or more of the following operations: delayed off, intermittent cycle scheduling, dimming, power monitoring, and accounting, and on/off control.

The control system 2400 in the illustrated embodiment includes a UV light power source 2432 (e.g., a UV-C power source) that enables UV intensity control and contact time control. The UV light source 460 may be any UV source capable of generating UV light at the target intensities, including UV-C light at the target intensities. The UV light power source 2432 may be capable of controlling current and/or voltage supplied to the UV light source 460, and may provide such power in a variety of ways. For instance, the UV light power source 2432 may supply power directly via wires to the UV light source 460, or the UV light power source 2432 may supply power wirelessly to the UV light source 460. In the wireless configuration, the UV light power source 2432 may include a primary capable of transmitting power wirelessly, and the UV light source 460 may include a secondary capable of receiving the wirelessly transmitted power.

The control system 2400 of this embodiment may include a controller 2436 capable of performing various functions pertaining to operation of the air treatment ceiling fixture. The controller can be a low current microprocessor configured on a regulated rail. The microprocessor can be configured to monitor temperature (e.g. ambient, source, and local microprocessor temperature), accelerometer values, voltage and current sensors, as well as any other suitable sensors for use in conjunction with a microprocessor, or any combination thereof. The microprocessor module can also allow for external communications and interface.

In the illustrated embodiment, the controller 2436 is coupled to a sensor system 2424 that provides the control system 2400 with various sensor inputs, such as passive infrared (PIR) sensors, motion sensors, and temperature sensors, and may provide an interface for RFID reader 2426. Other sensors are discussed throughout the disclosure that can be integrated with the disinfection control system 2400. The data collected by these sensors may assist in controlling operation of the control system 2400 and in collecting data that may be relevant to tracking infection-related events and controlling other UV air treatment units.

The sensor system 2424 in one embodiment may include a particle sensor capable of sensing information about particles present in the air that is external or internal, or both, with respect to the reactor chamber. The control system 2400 may vary in operation based on the particle information obtained from the particle sensor.

In one embodiment, the control system 2400 may be coupled to a cloud system also as described herein as a cloud based control system 2602. The cloud system 2602 may obtain multiple particle sensor readings for an environment, and direct fan speeds and on times to treat a plume of particulates within a larger environment of multiple devices (e.g., multiple air pathogen reduction systems) in a connected pathogen reduction system.

The controller 2436 in one embodiment may monitor the current and voltage of power supplied to the UV light source 460, and may determine whether the current and/or voltage are within preset ranges for proper operation and lamp diagnostics. UV light sources 460 can present open circuits, short circuits, or impedance changes causing different operating voltages. The controller 2436 may identify such conditions based on the current and/or voltage and send information pertaining to such conditions to a remote network component, such as a network server on the cloud, as a service request. In one embodiment, the UV light power source 2432 monitors the current and voltage to the UV light source 460 and feeds that information back to the controller 2436. The controller 2436 may also include volatile and and/or non-volatile storage memory. For example, the controller 2436 may include flash memory.

In one embodiment, the UV light source 460 and control system 2400 have integrated RFID capabilities. An RFID tag 2438 disposed on the UV light source 460 may allow the controller 2436 to uniquely identify the UV light source 460 using an RFID reader 2426. This allows the control system 2400 to properly validate the UV light source 460 and also allows new thresholds (e.g., operating currents and/or voltages and other operating parameters) to be transferred to the controller 2436 for the particular UV light source 460 connected to the air treatment ceiling system 400. These thresholds may change by manufacturer or lamp time and can also be changed over time as the controller 2436 adapts and learns the operating parameters of the UV light source 460.

The UV light power source 2432 in one embodiment includes an amplifier circuit, where an amplifier gain can be changed to increase or decrease intensity of the UV light source 460. The amplifier may change the voltage applied to the UV light power source 2432 to within allowed thresholds. It is noted that higher thresholds or operating near the upper end of a voltage range of the UV light source 460 may adversely affect the life of the UV light source 460. The operating intensity thresholds, operating ranges, or other operating conditions for the UV light source 460 may also be pushed and saved to the RFID tag 2438. For instance, the hours at each intensity level may be helpful to the controller 2436 as it may accumulate the time at each intensity for the UV light source 460 to enable total end-of-life calculations. This information may be persistent to the RFID tag 2438 of the UV light source 460 so that, if the UV light source 460 is associated with another air treatment ceiling system 400, that air treatment ceiling system 400 can be aware of operating parameters and an end of life associated with the UV light source 460.

Adjusting and applying power to the UV light source 460 at controlled intervals may allow the controller 2436 to control the UV power output. This may enable frequent in-and-out occupancy for the room area to be treatment compensated dynamically. It is not often ideal to run at the highest intensity as it impacts the UV light source 460 with shorter life. With a lower intensity operation, longer duration “on” cycle times (or dose times) may be targeted to obtain adequate disinfection.

Dynamic control may be utilized to increase dose momentarily during busy times. A running average of busy times and target dose changes can be preprogrammed and the controller 2436 may then modify these dynamically as presence iterations change with respect to the room area. This may be directed locally by the control system 2400, other UV air treatment units, or by a cloud interface or other network device via a communication protocol.

An exemplary control algorithm involves first having a setting of the target dose. Each air treatment ceiling unit may, for example, store a target dose in the form of intensity level and contact time at a calibrated distance for the room area. Fan characteristics can also be stored and the fan controlled accordingly (e.g., one or more RPM set points or frequency settings). A communication interface 2420 of the control system 2400 may be provided to receive information from and transmit information to external electronic devices. For instance, the communication interface 2420 may include a USB interface 2442 (or other wired communication interface, such as Ethernet or RS-232) or a BTLE interface (or other wireless communication interface) that can be configured to allow external electronic devices, such as a smartphone, tablet computer, or other mobile electronic device to automatically write UV parameters and other relevant values into the control system 2400.

Some UV light sources 460 are manufactured in glass rather than quartz and will not emit UV-C. The OEMs manufacturing the device can assure proper installation configurations over many mounting options and distances with a go-no-go answer for limits of performance. The expected lamp life also changes dynamically as these minimum intensity expectations are set. An aging percentage may be added to these numbers to account for source degradation over the expected source life. The dose data vs. power may be defined and measured in the lab first, stored and averaged over life and then verified at the surface in testing.

In some applications, additional security-related components may be provided in the control system 2400. For example, in one embodiment, a crypto chip 2444 may be included to provide each unit with a unique ID. Other mechanisms for identifying each air treatment ceiling system 400 may be provided. The security may also be augmented with a token and SSID for security purposes stored in non-volatile memory set up by installation staff through BTLE or USB program for WiFi interface. This crypto chip 2444 may be provided for an additional security measure and may be configured to create a disinfection and room occupation tracking device that can have the security conditions considered sufficient to write directly into an electronic medical record.

In one embodiment, the communication interface 2420 of the control system 2400 has BTLE and/or Mesh capabilities. The mesh network can be Zigbee or BACNet to meet specific regulatory requirements or hospital specifications. In extreme monitoring solutions a cellular module 2486 may be used to communicate the data to an external device (e.g., the cloud) as an alternative source of information gathering. As shown, the control system 2400 may include transceivers and antenna matching circuitry 2428 and a cellular module 2486 that are coupled to corresponding antennas 2452, 2450, 2454. The controller 2436 may also have ports to allow directed wired connections, for example, using USB, Ethernet and RS-232 protocols.

In some applications, the control system 2400 may have the ability to operate on battery power. The battery version may be provided with a battery 2448, which may be the power source for the air treatment ceiling system 400. The battery-based system may be chargeable in a variety of ways, including wired and wireless charging configurations. The power storage may be sized for UV dose and interval, and may be connected to charging equipment or directly chargeable. It may also have various indicators for providing feedback to a user.

As noted above, the UV light source 460 (e.g., UV-C lamp) may have an RFID tag 2438 and the control system 2400 may have an RFID reader 2426 to understand when the UV light source 460 has reached end-of-life to encourage appropriate use and maintenance. UV light sources 460 often have a life based on a number of hours as they self-destruct due to the nature of UV light, including UV-C light. The control system 2400, for example, through the controller 2436, may keep track of lamp “on time” by reading from and writing to memory resident on the RFID tag 2438. The control system 2400 may adjust the actual “on time” by a correlation factor to compensate for lamp intensity. For example, the control system 2400 may increment the lamp life counter by less than the actual “on time” for operation that occurs when the lamp intensity is reduced and may increase the lamp life counter by more than the actual “on time” for operation when the lamp intensity is increased. The correlation factor (or intensity adjustment factor) may be provided by the lamp manufacturer, may be determined through tests of the UV light source 460, or may be estimated based on past experience.

The communication interface 2420 of the control system 2400 may also have USB and Power over Ethernet (“POE”) circuitry 2437, which may enable usage without additional power cord requirements for this equipment. In one embodiment, the power management circuitry 2439 may allow inputs from power generating sources and various voltages enabling flexible power adaptation. For instance, the power management circuitry 2439 may allow AC power to pass through so that the host piece of equipment is undisturbed. When the air treatment ceiling system 400 is integrated into another electronic device, the power management circuitry 2439 may allow the air treatment ceiling system 400 to draw power from the power supply for the host electronic device as the power source. A single outlet can be used to avoid potential confusion when plugging in the device. The power management circuitry 2439 may be operable to power from a variety of sources, including wireless, USB, DC, and battery sources. In one embodiment the power regulation is done in a buck boost manner to provide an energy harvesting power supply that produces a regulated power source when voltage is produced by various power sources.

The control system 2400 in the illustrated embodiment may include regulator circuitry 2446 configured to facilitate operation of a UV light regulator. The regulator circuitry 2446 may include a motor controller and sensor circuitry. The motor controller and sensor circuitry may drive and monitor motor RPM of one or more fans. The motor controller may control the speed of the one or more fans, such as by adjusting a duty cycle of a PWM drive signal supplied to the one or more fans. The sensor circuitry may monitor current against a target and/or range of currents associated with a target RPM of the one or more fans.

In one embodiment, as discussed herein, the control system 2400 may include a room sensor interface 2455 operably coupled to the controller 2436. The room sensor interface 2455 may be configured to provide feedback indicative of whether the room area (potentially the entire area of the room) is occupied by one or more persons. The room sensor interface 2455 may be configured to count people or track the number of people within the room area. Alternatively, feedback from the room sensor interface 2455 may be used by a controller separate from the room sensor interface 2455 to count people or track the number of people within the room. The control system 2400 may use feedback from the room sensor interface 2455 to make various control decisions about how to control the UV air treatment system.

It is to be understood that the room sensor interface 2455 may be separate from the control system 2400 in an external device capable of communicating information indicative of presence of one or more persons in the room. For instance, the room sensor interface 2455 may be a motion sensor (e.g., a PIR sensor) capable of sensing the presence of one or more persons in the room or room area. This motion sensor may communicate wirelessly with the control system 2400 or with an intermediary device capable of relaying occupancy information to the control system 2400.

The control system 2400 may include a visible light driver 2445 separate from or provided in the visible light module 1842 (shown in FIGS. 18A-18J) to facilitate directing operation of a visible light source. The visible light driver 2445 in the illustrated embodiment may also include a user interface (e.g., an ON/OFF switch, a brightness adjuster, and a color adjuster) operable to allow a user to control operation of the visible light source. For instance, the user may utilize the user interface to direct the visible light driver 2445 to increase or decrease a color temperature of the visible light source. The visible light driver 2445 may include a controlled current source and/or a controlled voltage source to supply power to the visible light source in accordance with a target operative mode of the visible light source.

The control system 2400 of the present disclosure builds off of the control system disclosed in WO2021/138645 entitled System and Method of Disinfection to Baarman et al., filed on Jan. 2, 2021, which was previously incorporated by reference in its entirety. As discussed above, the control system of the present disclosure can include a physical connectivity interface (Ethernet/IP) so that the unit can be operated without a radio. The UV air treatment fixture of the present disclosure can be configured with a variety of DC power input options (48V DC (POE Compatible), Universal AC).

The control system 2400 can include microphone sensor input detection of ambient sound levels and people/activity sensing, active shooter detection using filters and triggers for grouping of data input, pressure sensor input to track changes in ambient pressure, used to calculate air velocities between units. Inputs for additional sensors (VOC/eCO2, PM) can be used to calculate or assist in calculating people loading and overall environmental health. Downlight power Control (e.g., On/Off or variable control) and automatic pathogen control (On/Off)—enable dynamic treatment mode can control power consumption while managing the pathogen loading of the environment. Fan Speed configuration can be provided to enable a dynamic treatment mode where the environmental loading calculation of 37 million bacteria per person per hour is used to drive variable treatment performance of the system by increasing fan speed as the system is designed for a range of fluence to dose pathogens effectively, as discussed in more detail in the dynamic control section later. The RFID tag within the UV source can be used to validate pathogen performance and time of replacement over the cloud Read/Write/Authenticate.

Configurable Operating and Management Modes. The UV air treatment units of the present disclosure can include a variety of different configurable operating and management modes.

The UV air treatment units of the present disclosure can include an auto clean mode that reacts to environment detection levels for dynamic treatment using a combination of one or more of sound, pressure, CO2, and VOC levels. The control inputs can be derived from one or more of a combination of local sensors on the UV air treatment unit, other UV air treatment units in the room or building, and Internet of Things devices. The various inputs can have different weightings and configurations that impact timing and configuration of auto-mode selection by the UV air treatment unit.

The various thresholds and configuration options associated with the auto clean mode can be preset with guard bands and programmable filter settings. While the various threshold and configuration options associated with auto clean mode may include factory presets, the control system can be configured to take calibration into account s that each UV air treatment unit has baseline settings specific to its environment.

The UV air treatment fixtures of the present disclosure can have a variety of different treatment modes. For example, the following different modes can be included Off-UV and Fans Off, Speed 1-UV On, Fans 6V (e.g., ˜50 CFM), Speed 2-UV On, Fans 8V (e.g., ˜65 CFM), Speed 3-UV On, Fans 10V (e.g., ˜100 CFM), Speed 4-UV On, Fans 12V (e.g., ˜115 CFM), Turbo Cycle-UV On, Fans 12V for preset Time (e.g., 1 hour).

In auto mode, each sensor or connector can act as a level set/hold trigger into the system to activate a clean cycle at the respective speed setting. These speed settings can be held at a desired cleaning cycle time length (e.g., settable between 1-240 minutes). The selfauto mode inputs can include sound (e.g., a predefined dBA threshold), dBA Level 1-40 dB, dBA Level 2-50 dB, dBA Level 3-60 dB, dBA Level 4-70 dB, dB Calculation-preset programmable, pressure change (delta psi, preset and programmable).

The various levels and thresholds (e.g., sound thresholds VOC ppm thresholds, etc.) can be configurable at setup during manufacture, or vary over operation life. That is, each UV air treatment unit can be configured with a baseline or set of baseline levels. Over time these baselines can change from the initial configuration based on sensor inputs or expected changes over the life of the unit. A moving average-type baseline can be used to influence the levels over time.

The various modes of operation can include certain triggers and control instructions for the UV air treatment system, for example:
Cleaning Cycle/Auto Mode Behavior, Fans can be configured to automatically transition to a certain speed settings or to turn off;
Sensors can be configured to periodically sample room environment and provide occupancy level or other information to the local control system or a network device
Fans can be configured for coordinated control according to a selected speed level for cleaning cycle timer.
Fans can be configured to automatically return to a predefined speed setting for the next cycle after being reconfigured based on a given trigger.
Dwell time setting for no occupancy cleanup can be programmable.
If Occupancy Level remains above a predefined level for a certain period, then the control system can control the UV air treatment system according to a room clean timer (settable to 60-240 minutes), after the system can enter a sleep mode (e.g., Fan/UV Off but other components such as certain sensors are active).
The control system can be configured for initialization and interface when multiple units are in proximity.
A level 1 trigger can be configured with a higher threshold than an adjacent unit(s) in turbo mode to avoid fan noise from unit triggering.
Connected Auto Mode.
Cloud can provide activity input to treatment mode.
Dynamic weight based on confidence of activity level in environment.
IoT Connector-Allow for Cloud Interface to influence Auto Mode Level Setting. In general, the UV air treatment fixture control system can base decisions on various local and remote stimuli such that multiple UV air treatment fixtures operation can be orchestrated to act in concert. However, the control system can also have settings in place to prevent external stimuli (e.g., from a cloud network or other UV air treatment fixtures) from over-influencing (e.g., the control system can prevent setting and configuration changes that would violate a maximum noise rule programmed into the UV air treatment fixture).

The UV air treatment unit of the present disclosure provides targeted treatment. The combination of various technologies (e.g., sensors, dynamic control algorithm, and variable fan speed) enable the targeting and reduction of pathogens in the air instead of indiscriminately cleaning and filtering. Source control can be activated by multiple sensors and system activity and can treat in target ways using formulas and algorithms based on airflow, known environmentally and clinically tested performance, settling times and variable system performance.

The UV air treatment units of the present disclosure can be network connected devices. That is, essentially any radio functionality can be integrated and utilized in its dynamic control algorithms and data reporting. For example the unit can utilize WiFi, Ethernet, sub-ghz, cellular, or other communication technologies to communicate among UV air treatment units, the cloud, and/or other network devices. While communication interfaces can be included in the units, they are not necessary and much of the functionality of various embodiments of the present disclosure can be implemented with no or limited radio functionality.

The UV air treatment units of the present disclosure can be integrated into various dynamic treatment systems such as those described in U.S. patent application Ser. No. 17/364,184 entitled Dynamic Treatment System and Pathogen Reduction Devices, filed Jun. 30, 2021 to Baarman et al. For example, the UV air treatment fixtures of the present disclosure can be part of a system providing a coordinated multi-level pathogen mitigation system and interface. The UV air treatment units of the present disclosure can augment the the various components and processes described to provide higher function control but reduce power and extend filter and source life by targeting treatment. For example, by utilizing control algorithms of the present disclosure to change fan speed dynamically and turn off after a dwell period for settling of various particles. The system can function locally and remotely with several levels on control interface and modification based in informational layers.

HEPA, UV, HEPA & UV or MERV 6+ and UV systems. Since the system is configurable, it can be configured for multiple modes of treatment based on the customer configuration and specification requirements. The system can be configured with filter and UV, filter only, etc. The filter quality can determine the back pressure and multiple configurations can be accommodated within the overall system platform.

Hub interface. The system may include a multiport hub or 5G hub mounted to the system to allow multiple units to connect within a space. This may be for converting protocols, providing security, enabling alternative communication pathways and protocols like fiber, 5G, 3G, 2.4 GHz to 5 GHz for ease of system installation and overall flexibility of configuration.

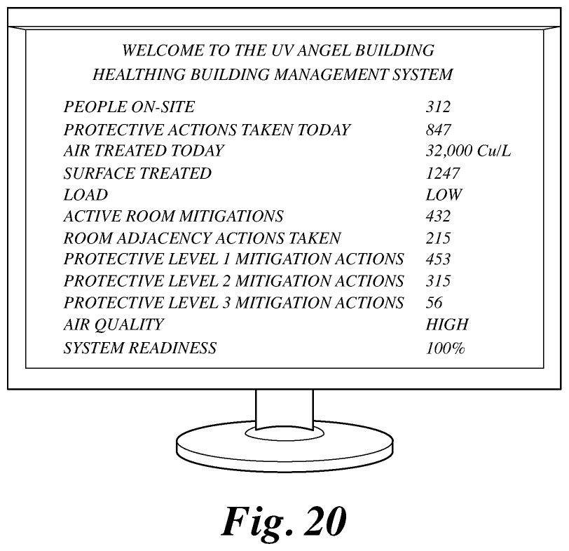

FIG. 20 shows an exemplary system health monitor that shows the present health of a building or monitored and mitigated area. The health monitor also functions as an assurance device by informing people of the safety and mitigation measures being applied within that environment. This assurance can inform, modify, and enhance behavior. Although a monitor interface is depicted displaying textual information, in alternative embodiment essentially any form of user interface can be utilized to provide the assurance information in graphical or textual form. For example, a dedicated assurance interface device can be mounted in a room, the assurance information can be communicated and displayed to a user's personal device (e.g., a tablet or cell phone) via a webpage or application. While the system health monitor can provide a simple and effective interface for viewing the status of one or more of the UV air treatment fixtures, it can also provide a convenient interface for instructing system or individual parameter changes or for initializing over the air updates to the firmware or other software on the various UV air treatment fixtures. Many of these datapoints can be utilized as additional inputs for the device, to better detect environmental load levels outside of the devices immediate zone.

Automatic pathogen reduction system with building performance tracking. The tracking of people, airflow, HVAC, air speed, pressures, VOC's, CO2, sound, occupancy sensing and active treatment at different levels can be used to display a building status. UV air treatment fixtures can reduce CFU's in the air by 56-85% and typically 56% on surfaces based on settling. In general, people are the most significant source of pathogens. People and typical building conditions are also the source of contamination. By reacting to these sources directly and dynamically, power, lamp and filter life can be preserved while operating to perform a specific job directly related to pathogen reduction in target areas.

Mobile or custom interface device and application. The mobile device application is a portal for set up, installation, on-line updates and programming. It can be the link to the cloud portal, or a hub can be used to collect data from units without radios. The BTLE link has a crypto security chip with a unique identifier. This assures security in data transfer and OTA (over-the-air) programming. The air system may have IRDA a two-way IR communications device that communicates through the indicator window or lighting lens and requires a lighting or USB adapter (dongle) for interface to the mobile device. This link can be used to reset the system, Test functions, configure communications, update software and settings, self-test, and download data.

### Power Management System

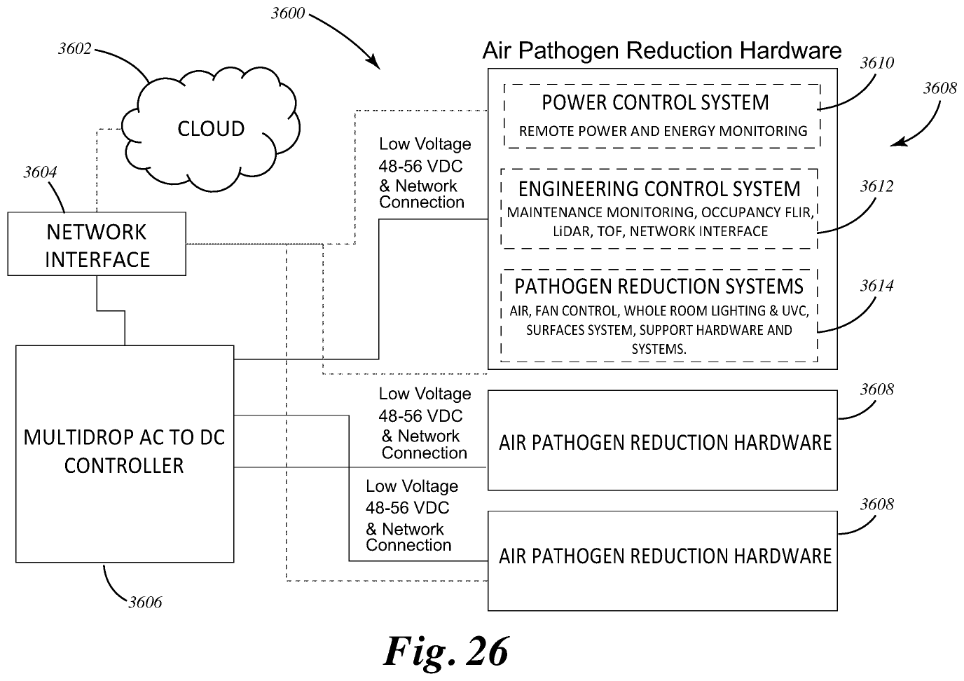

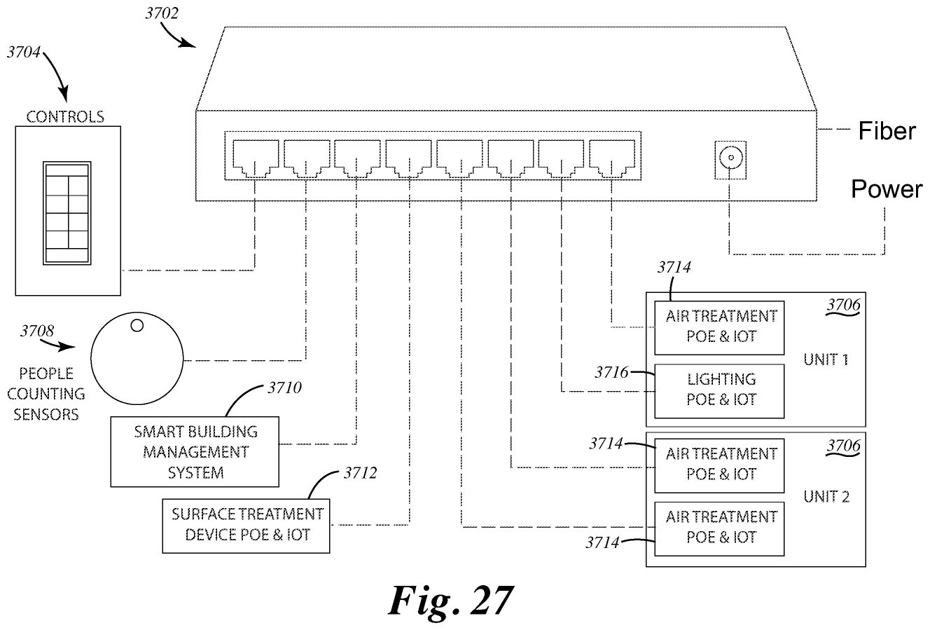

FIGS. 26 and 27 illustrate how UV air treatment fixtures, also referred to as air pathogen reduction hardware can be connected to a power management system. IN particular, FIG. 26 shows a connected pathogen reduction system accordance with one embodiment and FIG. 27 shows a connected pathogen reduction system accordance with one embodiment.

A power management system 2600, illustrated in FIG. 26, is provided in accordance with the present disclosure for controlling and powering the air treatment ceiling system. The air treatment ceiling system can include multiple air pathogen reduction hardware devices 2608. For example, separate air pathogen reduction hardware modules can be provided throughout a room. Each of these air pathogen reduction hardware modules can include one or more different systems therein, such as one or more power control systems 2610, one or more engineering control systems 2612, and one or more pathogen reduction systems 2614.

One example of a power control system 2610 that can be included in an air pathogen reduction hardware module is remote power and energy monitoring. The power control system can include one or more sensors, for example, current, voltage, power, or other type of sensor that can monitor the amount of power received, expended and report back to a control system, such as control system 2400 described in connection with FIG. 24. Local or remote lighting modules can be connected to a master disinfection control system, such as the disinfection control system of FIG. 24. Separate power and control wires can be connected to the disinfection control system. For instance, one of the air pathogen reduction hardware modules can be the disinfection control system of FIG. 24 and be coupled to other air pathogen reduction hardware, such as a portable lamp assembly via a multidrop AC to DC controller and/or a network interface, such as network interface 2702. As discussed herein, power over Ethernet can be utilized for communication and power connections, but in alternative embodiments, a wireless network connection among the air pathogen reduction hardware can be utilized or a wireless or wired network connection to a common server, such as a cloud-based server where control and data collection can be enacted as part of a cloud-based control system 2602.

Examples of engineering control systems 2612 include maintenance monitoring modules, occupancy forward-looking Infrared (FLIR) modules, light detection and ranging (LiDAR) modules, time of flight (TOF) modules, and network interface modules. These various engineering control systems 2612 can be included at the air pathogen reduction hardware to provide engineered control functionality. These modules are exemplary and other types of engineering control system modules can be provided, alone or in combination with other engineering control modules depending on the desired functionality of the air pathogen reduction hardware.

Examples of pathogen reduction systems 2614 that can be utilized in the air pathogen reduction hardware include one or more of air control, fan control, whole room lighting and UV-C disinfection, surface disinfection systems, support hardware and other various pathogen reduction systems. The pathogen reduction systems can provide disinfection functionality.

The air pathogen reduction hardware can be powered from a multidrop AC to DC controller 2606 that is connected to mains. A multidrop AC to DC controller can provide low-voltage differential swing multidrop connections. That is, a multidrop controller can provide power to a plurality of different air pathogen reduction hardware systems. The power can be provided through daisy chained connections of air pathogen reduction hardware or through parallel connections as depicted in FIG. 26.

In the current embodiment, the multidrop AC to DC controller converts AC power to 42-56 VDC power, or 48-56 VDC power, or another voltage level sufficient to power the air pathogen reduction hardware, and distributes the power to the air pathogen reduction hardware modules for operating power.

The multidrop controller can also provide network connections to the air pathogen reduction hardware over the low voltage network. That is, in some embodiments, the multidrop controller acts as a driver that can transmit and receive data to and from multiple air pathogen reduction modules simultaneously or in sequence. The multidrop controller can include a network interface or can be connected to an external network interface 2604 as depicted in FIG. 26. The network interface 2604 can connect to the cloud to provide Internet communication and IoT functionality to the air pathogen reduction hardware. For example, data can be collected and managed in a cloud-based service. Further, the air pathogen reduction systems can be controlled and monitored from a remote device that communicates with a cloud-based server or that communicates with the multidrop controller 2606.

The multidrop controller 2606 can provide various functionality in connection with the air pathogen reduction hardware. For example, the multidrop controller can monitor current, control scheme, balance between various parameters, energy control, and can manage communications. For example, the multidrop controller can connect to the air pathogen reduction hardware with DC copper or Ethernet POE and manage those connections.

One example of a network interface 2702 and associated topology that can be utilized in connection with a power management system of the present disclosure is illustrated in FIG. 27. POE generally describes any standard or ad hoc system that passes electric power along with data on Ethernet cabling. The network interface 2702 depicted in this embodiment has eight ports, five POE ports and three communication ports that provide communication but do not provide POE. In alternative embodiments, the network interface may have additional or fewer POE ports and communication ports. The network interface 2702 includes a power input that can be connected to mains power or another power source. The network interface 2702 also includes an inbound network connection, such as a fiber Internet connection that enables the network interface to communicate with cloud based services or with other remote servers or computers.

The POE network interface ports allow a single cable to provide both data connection and electric power to devices. In the depicted embodiment, power and communication can be provided to surface treatment devices 2712 and air pathogen reduction hardware units 2706, for example the depicted units that include an air treatment module 2714 and visible lighting module 2716. The POE connections can be provided as a supplement or instead of the multidrop controller connections. In some situations, certain devices may only receive power or may only receive communication. In other situations, all devices both receive power and are capable of communication over the network. The POE can be provided via IEEE 802.3 such as alternative A, alternative B, 4 PPoE standards, or essentially any other POE type protocol.

Via this network interface 2702, network connections can be provided to the various local devices, for example various devices located around a room. For example, several different combination air treatment and visible lighting units 2706 as well as surface treatment modules 2712 can be installed throughout a room and connected via POE in order to make each module a separate, individually addressable IoT device. The controls in the room 2704 can be programmed to control the certain designated devices in unison or to control one or more devices individually. The smart building management system 2710 can also be in communication with the system and can issue commands to the various devices via the network as well as receive reports regarding disinfection and other information available from the surface treatment devices 2712, combination units 2706, sensors, controls, or any other equipment connected to the POE network interface 2702.

The network interface can be connected to various sensors, such as a people counting sensor 2708 that can count the number of people in proximity of the sensor. The tracking information can be relayed through the network interface to a cloud server. The data can be utilized to improve disinfection and disinfection cycle interruption recovery strategies.

### Dynamic Control System

The UV air treatment fixture, also referred to as a dynamic air mitigation system, can use one or more methods to influence and mitigate biological or exposure conditions. The control system can be configured to track and manage various metrics and levels for a better understanding of impact and control within environments. The control system can define and enable healthier environments creating a more complete automated health management system. The system can assist in tracking of relevant data and exposures while managing and mitigating pathogens in the environments.

A control system in accordance with the present disclosure can utilize one or more sensor systems for various purposes.

A sound sensor, such as a microphone can be disposed on each UV air treatment fixture to enable collection of sound data. The microphone can be utilized to trigger various monitoring conditions. The control system of the UV air treatment fixture can control the fan. For example, based on sound information the fan can be turned on, off, or its speed can be dynamically changed based on the sound information. The system can be configured to collect sound data and adapt the noise the unit produces based on the conditions in the unit and room.

Room sound level and ambient thresholds can inform a dynamic algorithm of activity levels (e.g., see activity levels in Table 4) to dynamically adjust the expected Quanta by speaking levels. This measurement can be performed with all systems off and by each individual unit. The units in a room can sequentially power up individual units within a room while all systems listen to each unit identifying sound per unit. Table 4 shows the definitions of various Quanta on exhalation volume enhancement rates by activities.

TABLE 4

| | Enhancement |
|---|---|
| Activity | Rate |
| Resting, Oral breathing | 1 |
| Resting, Speaking | 4.7 |
| Resting, Loudly speaking | 30.3 |
| Standing, Oral breathing | 1.2 |
| Standing, Speaking | 5.7 |
| Standing, Loudly speaking | 32.6 |
| Light exercise, Oral breathing | 2.8 |
| Light exercise, Speaking | 13.2 |
| Light exercise, Loudly speaking | 85 |
| Heavy exercise, Oral breathing | 6.8 |
| Heavy exercise, Speaking | 31.6 |
| Heavy exercise, Loudly speaking | 204 |
| Quanta exhalation per person per minute | 26.35 |

Room Spectral Sound Content refers to a spectral response from measuring key frequency components using a microphone and applying spectral filters. The filters can be FFT based on bearing failures, filter pressure thresholds and various failure modes trained to the system. Other events like gunshots, speaking, fan settings, door opening, and closing can also be trained and measured. Both as a single unit sensor and as a room with multiple units monitoring sensor events and thresholds utilizing a unit control and cloud interface.

A pressure sensor can be included on one or more of the UV air treatment fixtures. The pressure sensor can measure pressure to see individual unit pressures for calculating room level differences in order to calculate air velocities between units, rooms and other adjacencies.

A particle count sensor can be included on one or more of the UV air treatment fixtures. Particle count can be used as a measure of air quality and can be linked to people loading in an area.

A temperature sensor can be included on one or more fo the UV air treatment fixtures. Temperature can be used to determine room air mixing and viability of pathogens over a temperature range. Temperature can also be used to determine temperature degree days in conjunction with humidity to determine fungus sporulation timing. Proper temperature and humidity ranges can also determine risk of extended pathogen life. Measured temperature values can be used to determine building health and risk of exposure.

A humidity sensor can be included on one or more of the UV air treatment fixtures. Humidity is an indicator of the ability of a pathogen to reproduce or survive. Using a pathogen look up table stored in memory, the expected life of aerosolized pathogens based on temperature, humidity, and changes can be determined.

A CO2 sensor can be included on one or more of the UV air treatment fixtures. CO2 is an air quality indicator, circulation indicator, mixing indicator and people loading indicator. Exposures at various rates can assist in determining health exposures and improved air quality practices for building control feedback. CO2 exposure may be used to enable additional fresh air intake helping to control the HVAC fresh air intake actuator for that purpose. The HVAC system may also step up the air change rate in response to the CO2 sensor output provided by the UV air treatment fixture. CO2 levels can also be used to classify human activity within the room, since CO2 exhalation follows a similar pattern to the quanta exhalation factors listed in Table 4 above.

An O2 sensor can be included on one or more of the UV air treatment fixtures. O2 is an air quality indicator and a fresh air mixing indication for building health and exposure levels.

A volatile organic compound (VOC) sensor can be included on one or more of the UV air treatment fixtures. A VOC measurement can be an indicator of building health and thresholds can be set that indicate exposure limits and health risks. Readings from the VOC and other sensors of the UV air treatment system can be provided to a disinfection portal or other network device for defining and creating a healthier environment. For example, the UV air treatment fixtures of the present disclosure can communicate and coordinate with a disinfection portal and disinfection tracking network such as the one disclosed in the Disinfection Tracking Network WIPO publication, WO2021/183600, filed on Mar. 10, 2021 to Baarman et al., which is hereby incorporated by reference in its entirety. A mobile or other application can report personal impact or side effects of environmental exposure. VOC exposure may be used to enable additional fresh air intake helping to control the HVAC actuator for that purpose.

An occupancy sensor system can be included on one or more of the UV air treatment fixtures. The occupancy sensor system may include one or multiple sensors. Occupancy can be measured by audible talking in the room, movement, proximity, or a combination. Movement can determine activity that also informs the Quanta chart (see Table 4) which in turn can drive the dynamic control algorithm for the UV air treatment fixture. The use of an IR camera is one reliable occupancy solution while passive infrared and radar/lidar sensors can also act as occupancy sensors tracking people moving in and out of a space allowing a constant inventory of people counting in a space. The number of people can be used to drive one of the inputs in a dynamic control algorithm for one or multiple UV air treatment units working in conjunction with each other. The movement of people can also be used as it relates to Table 4 to determine potent elevation of the Quanta of pathogens.

The fan system of the UV air treatment system can include one or more variable revolution per minute (RPM) fans that can each be controlled to spin at a selected RPM. The one or more fans can be driven at a target RPM to drive airflow at a desired velocity (e.g., a specific cubic feet per minute (cfm)) requested or set by a dynamic control algorithm. The one or more fans can be driven as an analog value making changes in speed discrete. The system also can identify the RPM of each unit as it relates to the sound levels of each unit.

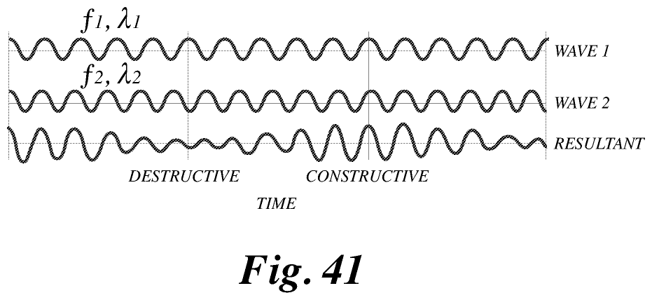

FIG. 41 illustrates how overall system sound can be managed by varying the RPM of frequency components of the one or more fans. The system can measure when the overall system noise is additive or normal seeking to manage or minimize the noise profile of the system of units. The setup or calibration process measures and offsets the RPMs of units in each room to find the lowers sound profile using this data. Those values can then be used as offsets for the low and high RPM levels and stored in memory.

Filter life can be tracked based on sound levels and potential sound differences. By utilizing a table of sound levels at various filter loading scenarios, the filter life can be identified and tracked. The system can track particulates, fan speed and on time by referencing a predefined table. The predefined table of sound levels used in reference to the filter verify the state of filter life.

Biological exposure in a room can be derived by the Quanta as a system. Table 4 shows an example of various data that can be collected from each UV air treatment unit. The particular data collected is merely exemplary, in other embodiments, additional, fewer, or different information can be collected from sensors included in the UV air treatment unit(s). The people counting in a room, the activity and sound level all can be used as dynamic factors taken into consideration to drive a pathogen mitigation control algorithm for one or multiple UV air treatment fixtures. Although removal is taking place the exposure levels remain at the calculated Quanta within the room. That exposure level can be tracked as an exposure rate to track potential infection rates and mitigation protocols. This may also drive additional levels of expected performance optionally stepping up the CFM earlier (e.g., when people enter a room) and higher RPM/CFM based on these historic risk levels allowing optimizing room level mitigation performance. This may also be built in or selected as a system performance target. Further, a building maintenance system or operator may interact with the UV air treatment system to control noise in the space while in use.

TABLE 5
Dynamic System

| | |
|---|---|
| Monitoring voices/events | Loudly speaking |
| Monitoring people movement | Light exercise |
| Pressures and velocities | Low |
| Indoor Air Quality | Low |
| Mixing | Low |
| Outside Air Quality | Low |
| Temperature | Ideal |
| Humidity | High |
| Fungus spoulation probability | Med |
| Monitoring CO2 | Ideal |
| Monitoring VOC's | OK |
| Monitoring sound/Frequency | 42 dBA |

In essence, the dynamic control system of the air mitigation system of the present disclosure attempts to maintain a total pathogen removal rate for the room that is greater than the rate at which pathogens are introduced. The system uses quanta estimates based off of 2019 Coronavirus, which is known to be more transmissible than many other serious infections, thereby introducing a factor of safety into the analysis. This is set forth with the equation:

Qin≤Qout

Qᵢₙ can be estimated based on number of people and estimates about the rate at which they emit quanta of pathogens. It is estimated that each person emits quanta at a base rate of about 18.6 quanta per hour. This rate can then be adjusted based on activity level by multiplying by the enhancement rates found in Table 4 above.

### Table 6

By tracking the number of people in the room and estimating the type of activity each is engaged in, a quanta emission rate can be assigned to each person. The sum of these emission rates is then Qᵢₙ.

Qₒᵤₜ is the total removal rate of quanta, which can come from a variety of sources in a given room. All of these sources contribute to the total equivalent clean air delivery rate (CADRₑ), which is then related to the total quanta removal rate by an exponential function:

$${Q}_{out} = Q ( 1 - exp ( - \frac{{CADR}_{e}}{V} ) )$$

$$CADRe = ( CADRAH + CADRDC + CADRDP + CADRUVA ) * ϵ M$$

V is the room volume. CADR_{AH} is the CADR of the air handler (filter efficiency times volume flow rate).

CADR_{DC} and CADR_{DP} are the equivalent CADR of microorganism decay and deposition respectively. For this algorithm, based on coronavirus, 46%/hour and 26%/hour are the estimates of decay and deposition respectively. The CADR rates that would be equivalent to these inactivation rates are given by

$$- \frac{ln ( 1 - r ) * V}{60}$$

Where r is the rate of decay/deposition, and 60 is a unit constant to convert from decay/deposition per hour to CADR in cubic feet per minute.

CADR_{UVA} is the sum of the CADR values from each UV air treatment unit in the room or area, which will be the product of each unit's volume flow rate times the single-pass efficiency of each unit. Single-pass efficiency is assumed to be 100% to four significant digits for most organisms, but can be adjusted as new pathogens of interest develop.

ϵM is the mixing efficiency of the room. UV air treatment units in accordance with the present disclosure and air handlers both influence the value of ϵM, so the algorithm can be configured to estimate and adjust this value based on whether the air handler is on as well as the expected impact of changing flow rates through UV air treatment units.

When no one is present in the room and quanta levels are below a threshold value, the UV air treatment units can be configured to turn off or enter a low power mode. When people are present in the room, but are estimated to be introducing fewer quanta than the maximum amount that can be removed (Qᵢₙ<Q_{out,max}) the flow rate of UV air treatment units can be solved for by setting Qₒᵤₜ equal to Qᵢₙ (the condition where pathogen load in the room will not increase over time), and rearranging the equations to obtain:

$${CADR}_{UVA} = - ln ( 1 - \frac{{Q}_{in}}{Q} ) * \frac{V}{{ϵ}_{M}} - ( {CADR}_{AH} + {CADR}_{DC} + {CADR}_{DP} )$$

When people are present in the room and estimated to be introducing more quanta than the maximum amount that can be removed (Qᵢₙ>Q_{out,max}) the UV air treatment units can be configured to run at a predetermined mode with higher or maximum speed while integrating the current excess quanta (Qᵢₙ−Q_{out,max}). Once the estimated quanta emission rate has decreased to less than the maximum removal rate (Qᵢₙ<Q_{out,max}) the units can be configured to continue to run at an elevated flow rate until estimated quanta levels are below the threshold value.

Each biological exposure condition can be tracked and reported to a network device, an exposure notice can also be sent to the building operator that a room has become an infection hazard or has reached over capacity. The room may also have an air quality monitor that defines the risk within a room as capacity changes or environmental conditions change impacting the removal or extended pathogen life. This can take the form of a physical interface device or be communicated by the system to a mobile or other device. This determination of Quanta can also define the room capacity as the dynamic conditions within the room are monitored.

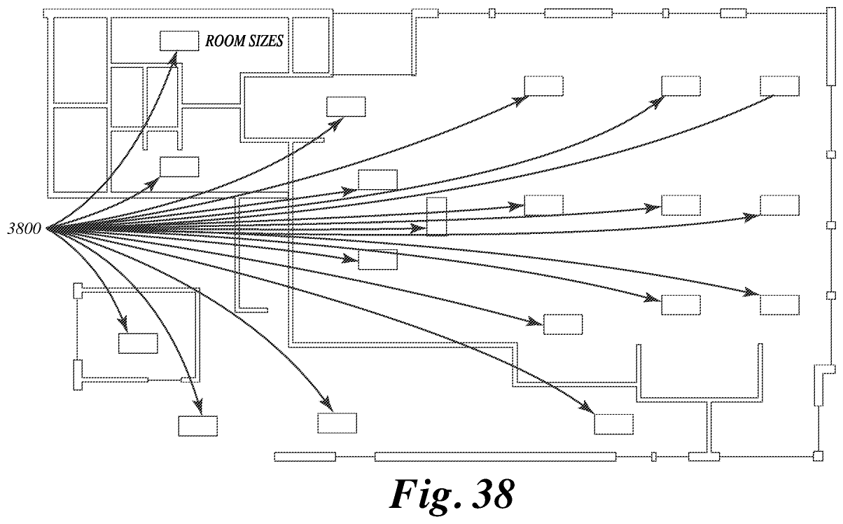

FIG. 38 illustrates an exemplary building layout with multiple UV air treatment fixtures 3800 installed. In this embodiment, each fixture can collect and be controlled based on various information. The units can collect information relating to: room spectral sound content, unit spectral sound content, pressure, particle count, temperature, humidity, CO2, O2, VOCs, occupancy, fan RPM, and filter life, to name a few examples of the types of information that these units can collect. Further the units can be individually or collectively controlled (e.g., individual unit sound can be turned on and off and multiple units can be turned on or off to control room sound). The systems and the data collected from each unit can be used to inform environmental health data and mitigate pathogen exposure.

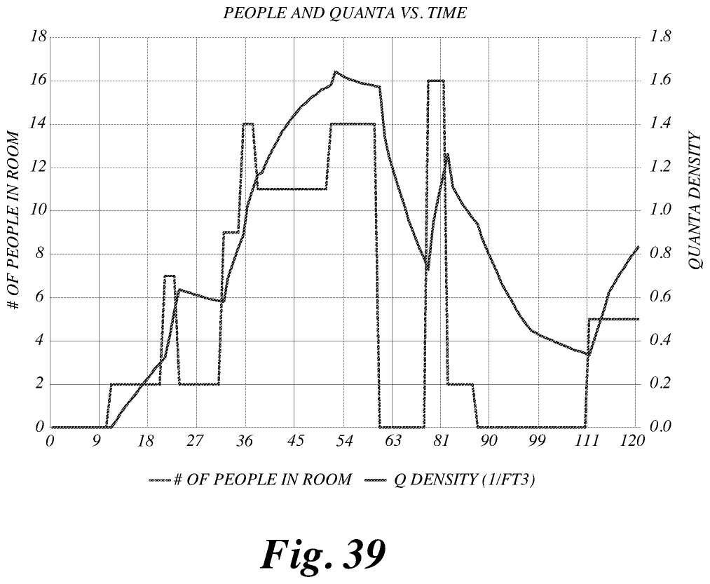

FIG. 39 shows an exemplary graph of calculated Quanta of pathogens as it relates to the number of people changing in a room. The dynamic calculation uses the room configuration and activity within the room to set the fan speeds for desired pathogen mitigation.

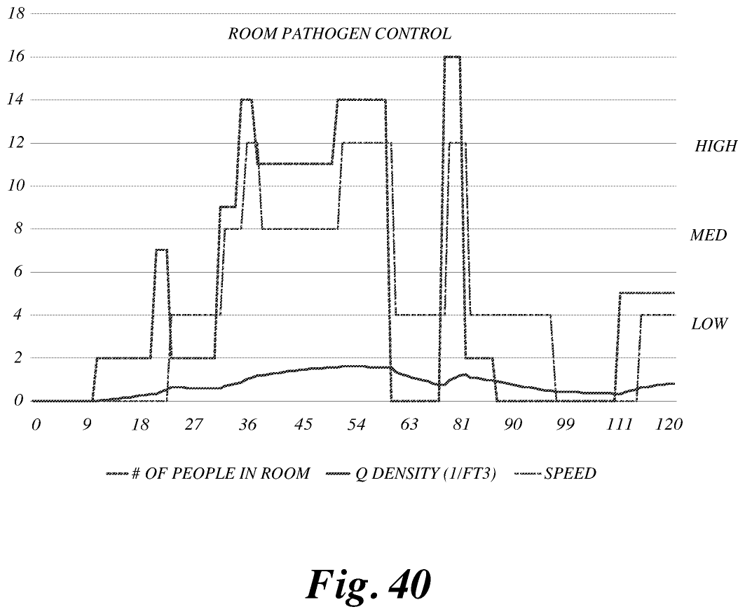

FIG. 40 shows an exemplary graph of the fan speed controlled by the Quanta and change in people in a room. It also determines the time and speed needed to mitigate the remaining Quanta when people leave a room. This allows the system to conserve energy by shutting off or entering a low power mode as the Quanta is mitigated after some on time. Then the system remains off or in low power mode until people are detected, the HVAC or pressure changes indicating air movement that can loft pathogens. The on time is reset and the system begins mitigating pathogens in the room again.

FIG. 41 shows a graphic illustrating sound minimization. The UV air treatment units can work together in sequences to adjust fan RPM or frequency to prevent additive or constructive sound waves. By offsetting the different UV air treatment unit fan frequencies, the amount of constructive sound waveforms can be reduced to manage the total noticeable sound in the room.

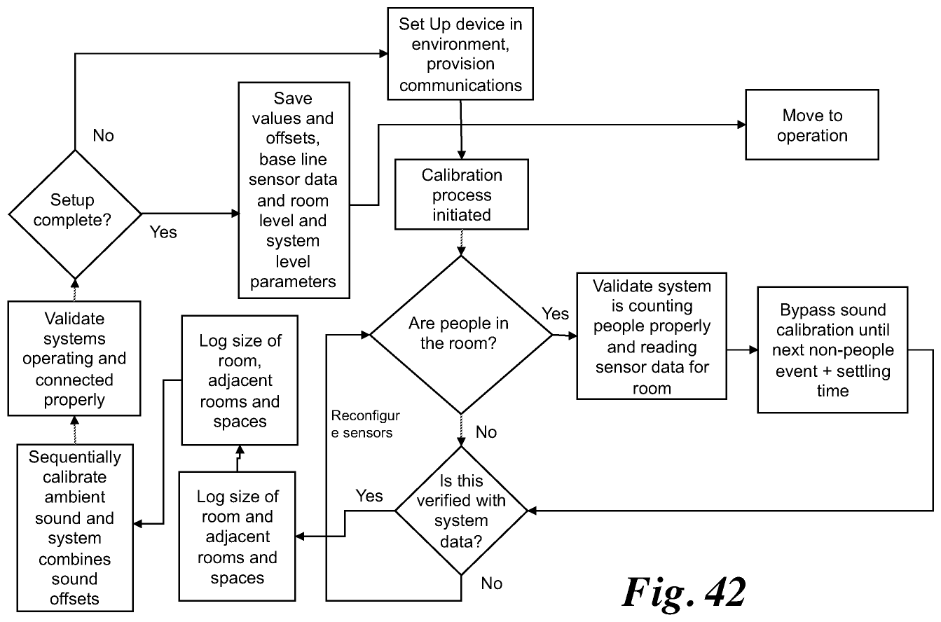

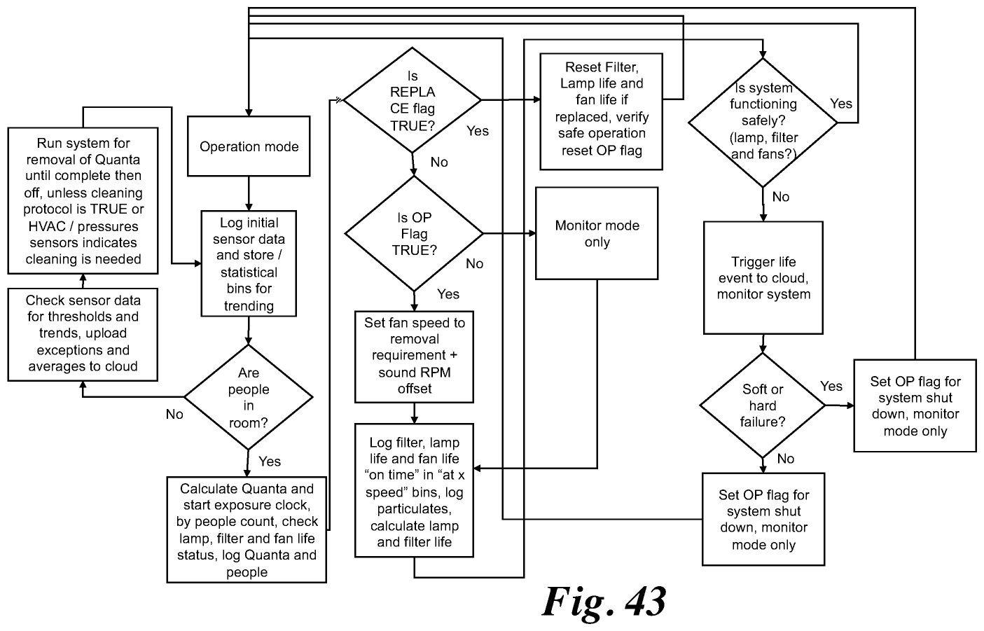

FIG. 42 illustrates an exemplary process flow for a sound and people calibration process for initial system set up and optimization. FIG. 43 illustrates an exemplary operational flow chart for operation and data gathering of a UV air treatment unit.

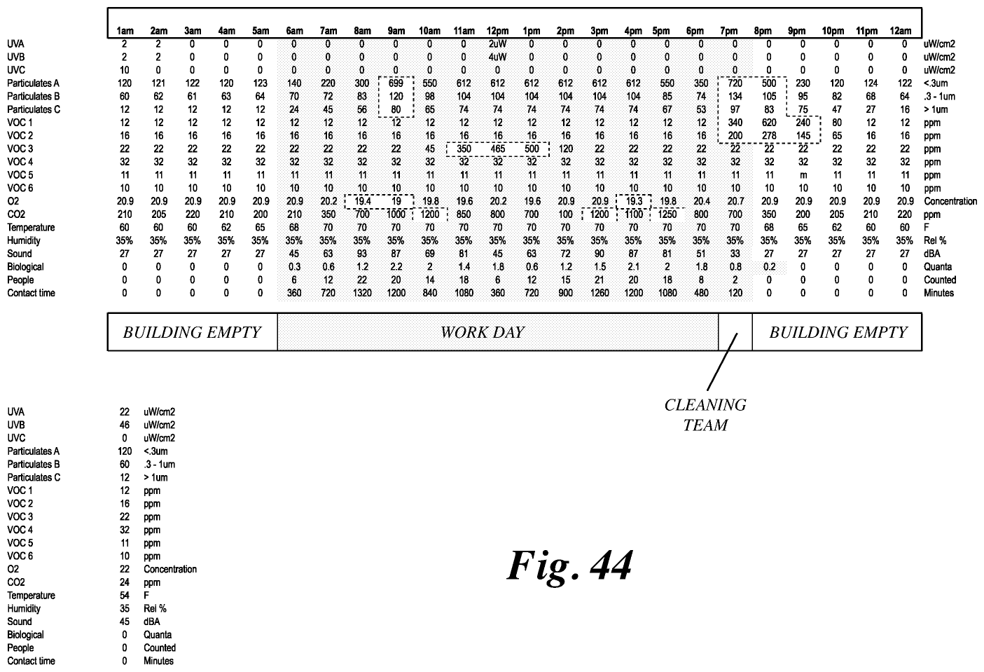

FIG. 44 illustrates an exemplary sensor data daily table.

### Configurable Maintenance Door

A typical dropped ceiling or suspended ceiling has a gridwork of metal channels in the shape of an upside-down “T”, also known as a ceiling T-rails. The ceiling T-rails are suspended on wires from the overhead structure or the true ceiling. The ceiling T-rails snap together in a regularly spaced pattern of cells known as a grid or suspension grid to form a ceiling T-rail system. There are a variety of different suspension grid types with varying cell sizes. In the U.S. and some other countries, the cell size in the suspension grids is typically either 2 by 2 feet (610 mm×610 mm) or 2 by 4 feet (610 mm×1,220 mm), and the ceiling tiles and light fixtures are the same size to provide an aesthetically pleasing appearance. In Europe and some other countries, the cell size in the suspension grids is 600×600 mm or 600×1,200 mm, while the ceiling tiles and light fixtures are slightly (5 mm) smaller at 595×595 mm or 595×1195 mm. Lightweight ceiling tiles or “panels” drop into the grid to fill each cell in the ceiling T-rail system.

A UV air treatment ceiling system as described above may be installed in a ceiling T-rail system. The air treatment ceiling system may alternately be referred to as an air disinfection system. The UV air treatment ceiling system can be installed in the plenum space using z-axis installation. There is limited plenum space between the T-rails and the true ceiling. After the UV air treatment ceiling system has been installed, periodic maintenance of the system will be performed. It is desirable to access the UV air treatment ceiling system for maintenance without having to go through the plenum space or remove the system from the suspended ceiling.

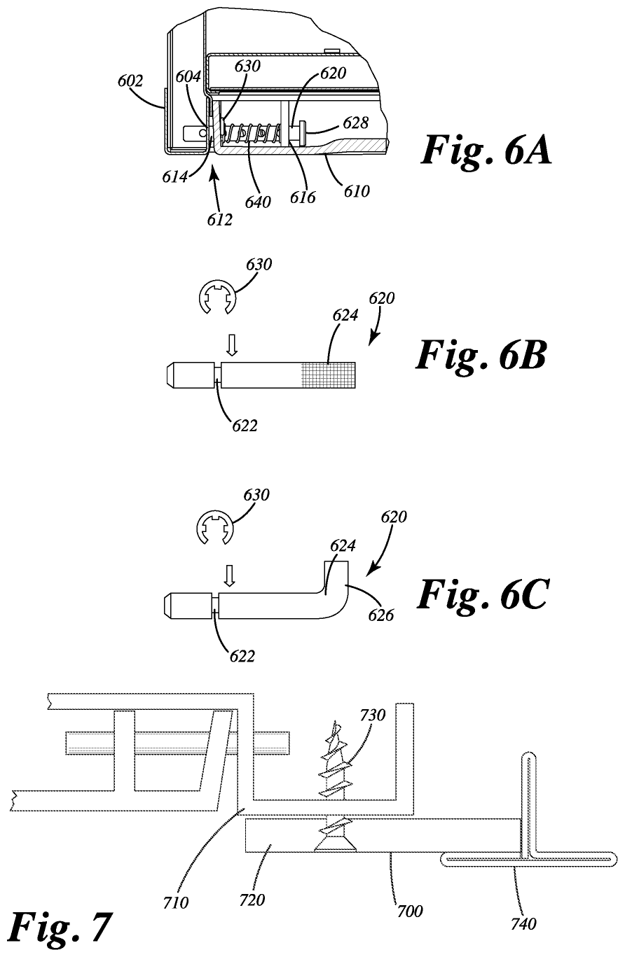

One way to provide such access is to have a configurable maintenance door on the front of the UV air treatment ceiling system. FIG. 17 shows an air treatment ceiling system 1700 with a configurable maintenance door 1710. In one embodiment, the configurable maintenance door 1710 can be made from plastic. The configurable maintenance door 1710 may be movably coupled to the air treatment body 1702 of the air treatment ceiling system 1700 through spring clips, a hinge, or other door actuators (not shown in FIG. 17.) The door actuator (not shown in FIG. 17) can be any suitable coupling mechanism. One exemplary door actuator is shown in FIGS. 6A-C and other exemplary door actuators are shown in FIG. 56B as torsion springs 5302, which are discussed in more detail below in connection with that embodiment. The configurable maintenance door 1710 may be configured to provide or facilitate access to the air treatment body 1702.

FIG. 30C shows a cross-sectional view along the line 30C-30C of an air treatment ceiling system 3000 of FIG. 30B according to one embodiment. Various embodiments of the air treatment ceiling system are described above in more detail. The air treatment ceiling system 3000 has a configurable maintenance door 3010. The air treatment ceiling system 3000 includes an inlet chamber 3050, a reactor chamber 3020, and an outlet chamber 3030. The reactor chamber 3020 can include a germicidal light source 3060 operable to generate UV light. The germicidal light source 3060 may alternately be referred to as a UV light source. The reactor chamber 3020 may have an untreated air inlet 3022 and a treated air outlet 3024. The inlet chamber 3050 can be fluidly connected to the reactor chamber 3020 through the untreated air inlet 3022. The outlet chamber 3030 may be fluidly connected to the reactor chamber 3020 through the treated air outlet 3024. The reactor chamber 3020 can define a reactor chamber opening 3026. The reactor chamber 3020 has an air treatment region 3028 that is operable to receive air from the untreated air inlet 3022 and direct air to the treated air outlet 3024. UV light from the germicidal light source 3060 may be directed to the air treatment region.

The inlet chamber 3050 can define an inlet chamber opening 3056. As depicted, the inlet chamber 3050 includes two collimators 3040. In an alternate embodiment, the inlet chamber 3050 can include at least one collimator 3040. The inlet chamber 3050 may include a filter 3080 located proximal to the inlet chamber opening 3056. The filter 3080 can be configured to block certain debris from entering the air treatment ceiling system 3000. In one embodiment, the filter 3080 may be a MERV 6 filter. In an alternate embodiment, the filter 3080 may be a HEPA filter. In yet another embodiment, the filter 3080 can be any type of filter suitable for the application. The outlet chamber 3030 can define an outlet chamber opening 3036. As depicted, the outlet chamber 3030 includes two collimators 3040. In an alternate embodiment, the outlet chamber 3030 can include at least one collimator. The outlet chamber 3030 may include a fan assembly 3070. The configurable maintenance door 3010 can be configured to span the inlet chamber opening 3056, the reactor chamber opening 3026, and the outlet chamber opening 3036. In one embodiment, the configurable maintenance door 3010 may be configured to span the entire front surface of the air treatment ceiling system 3000.

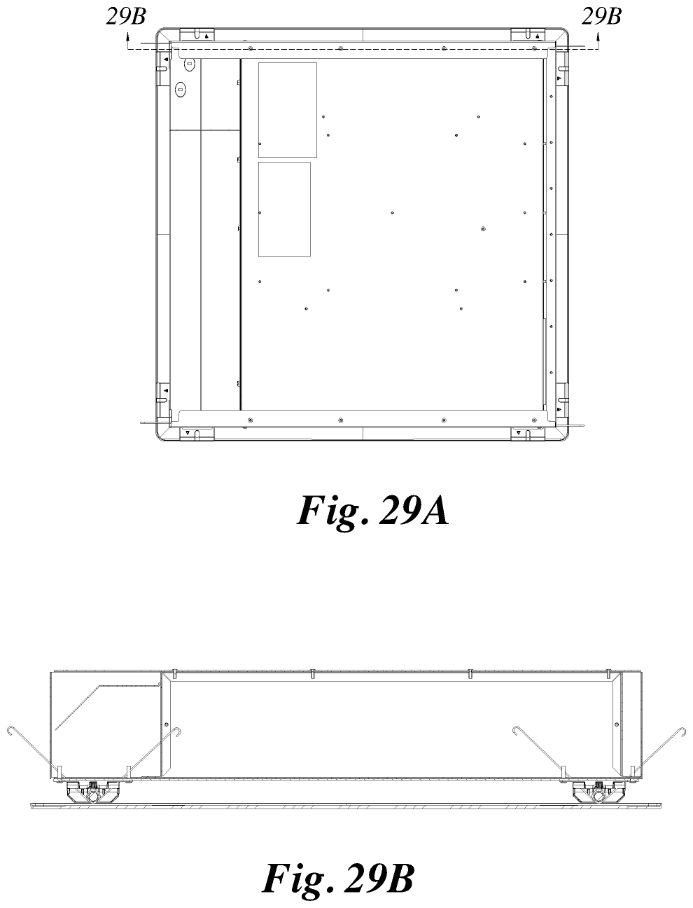

FIG. 29A is a top view of an air treatment ceiling system according to one embodiment and FIG. 29B is a sectional view of the air treatment ceiling system of FIG. 29A along the line 29B.

FIG. 6A is a detailed view of an alternative mounting system for a configurable maintenance door. The hinge 612 is shown coupling a configurable maintenance door 610 to the air treatment body 602 of the air treatment ceiling system. As depicted, the configurable maintenance door 610 is rotating out of the page toward the viewer. In one embodiment, the air treatment body 602 of the air treatment ceiling system may be made from sheet metal. As depicted, the hinge 612 includes a pin 620, a clip 630, and a compressible material 640. As depicted, the pin 620 may have a head 628 that can provide increased grip of the pin 620. As depicted, the compressible material 640 is a spring. In an alternate embodiment, the compressible material 640 can be any other suitable compressible material. The pin 620 may be inserted through three openings. First, the pin 620 can be inserted through a second hinge opening 616 and a first hinge opening 614 in the configurable maintenance door 610. Then, the pin 620 may be inserted through an opening 604 in the air treatment body 602 of the air treatment ceiling system. The compressible material 640 may surround the pin 620 between the first hinge opening 614 and the second hinge opening 616 in the configurable maintenance door 610. The clip 630 can be adjacent the compressible material 640 and at least partially surround the pin 620. The clip 630 may hold the compressible material 640 between the clip 630 and the second hinge opening 616 in the configurable maintenance door 610. Put another way, the clip 630 can compress the compressible material between the clip 630 and the second hinge opening 616 in the configurable maintenance door 610.

The hinge 612 can be removed without vertical access to the air treatment ceiling system, just as the vertical installation clips 1720 described in connection with FIGS. 57-65. This allows the configurable maintenance door 610 to be removed or replaced without having to remove the air treatment ceiling system from the ceiling T-rail system. Vertical access can be defined as access to the air treatment ceiling system in the plenum. For example, accessing the sides or back of the air treatment ceiling system when the air treatment ceiling system is installed in a ceiling T-rail system uses vertical access. As depicted, the pin 620 may be removed from the opening 604 in the air treatment body 602 of the air treatment ceiling system and the first hinge opening 614 and the second hinge opening 616 in the configurable maintenance door 610. The configurable maintenance door 610 is no longer connected to the air treatment body 602 of the air treatment ceiling system.

FIGS. 6B-6C show two exemplary embodiments of the pin 620. The pin 620 may contain a channel 622 for the clip 630. As shown in FIG. 6B, the pin 620 may have a textured portion 624. When used as part of the hinge 612, the textured portion 624 can extend beyond the second hinge opening 616 into the configurable maintenance door 610 to provide improved grip of the pin 620. In one embodiment, the textured portion 624 may be knurled. As shown in FIG. 6C, the pin 620 may include an angled portion 626. When used as part of the hinge 612, the angled portion 626 may be located beyond the second hinge opening 616 into the configurable maintenance door 610. The angled portion 626 can allow improved grip of the pin 620. The hinge 612 allows the configurable maintenance door 610 to rotate to provide access to the UV reactor chamber to replace the lamp and filters as well as other air treatment ceiling system maintenance.

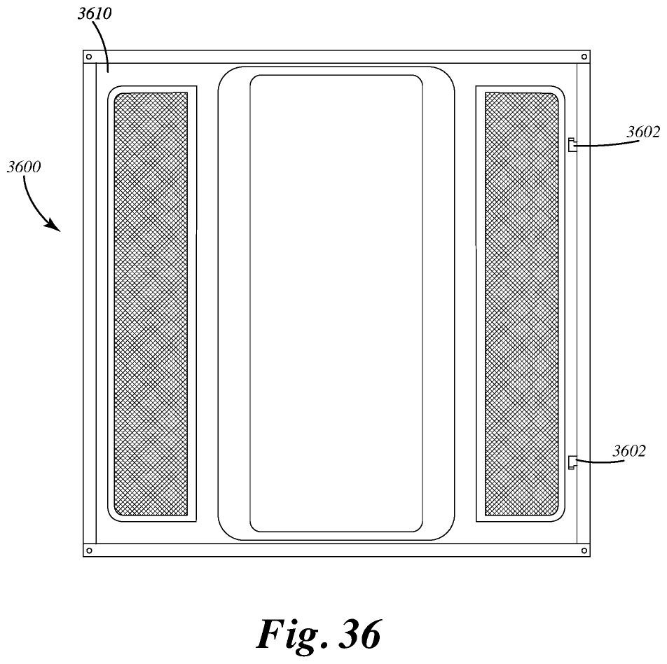

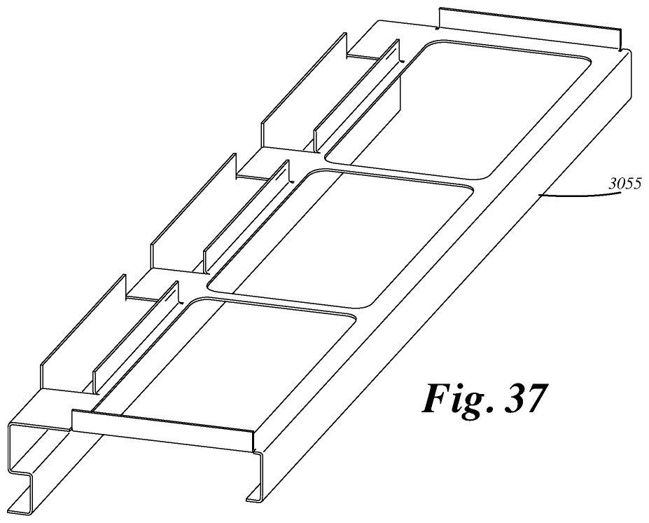

Returning to FIG. 4A, the reactor access door 428 may be secured in a seated position using one or more thumbscrews 431, latches, or other fasteners. The seated position can alternately be referred to as a closed position, a secure position, or a latched position. The thumbscrew 431 may selectively secure the reactor access door 428 to the air treatment body 1702. In one embodiment, the fastener can be a plastic spring tab that acts as a latch and can be pinched to break the contact between the access door 428 and the air treatment body 1702 to allow the access door 428 to open. Any type of suitable fastener may be used to secure the access door. In one embodiment, the fastener can be a hook and loop connection. In one embodiment, a magnetic connection. When the fastener is unlatched, the access door 428 is in an unseated position. The unseated position may alternately be referred to as an open position or an unlatched position. The access door 428 may be in a variety of positions with respect to the air treatment body 1702 and be in the unseated position. FIG. 36 depicts an alternative embodiment of a configurable maintenance door 3610 installed on an air treatment body of a UV air treatment fixture. It includes latches 3602.

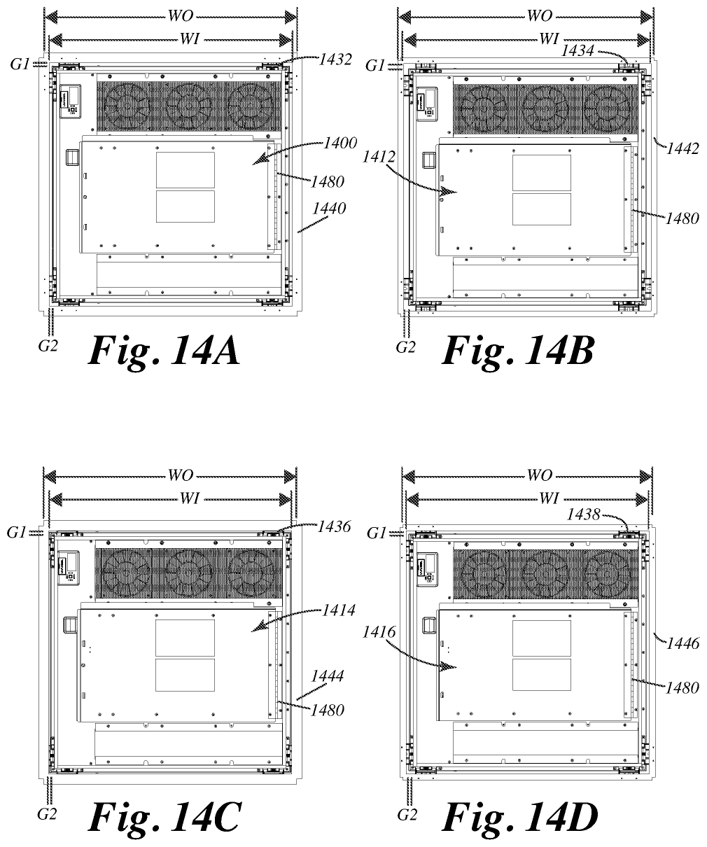

The reactor access door 428 can be moveable between the seated and unseated positions through the at least one hinge 1480 (See FIGS. 14A-D). In one embodiment, the reactor access door 428 may be moved through the manual input of a human. In another embodiment, the it may be operably coupled to an actuator capable of moving the door between the seated and unseated positions. When the access door 428 is in the unseated position, it can provide access to the UV light. More broadly, in the unseated position, the reactor access door may provide access to the reactor chamber.

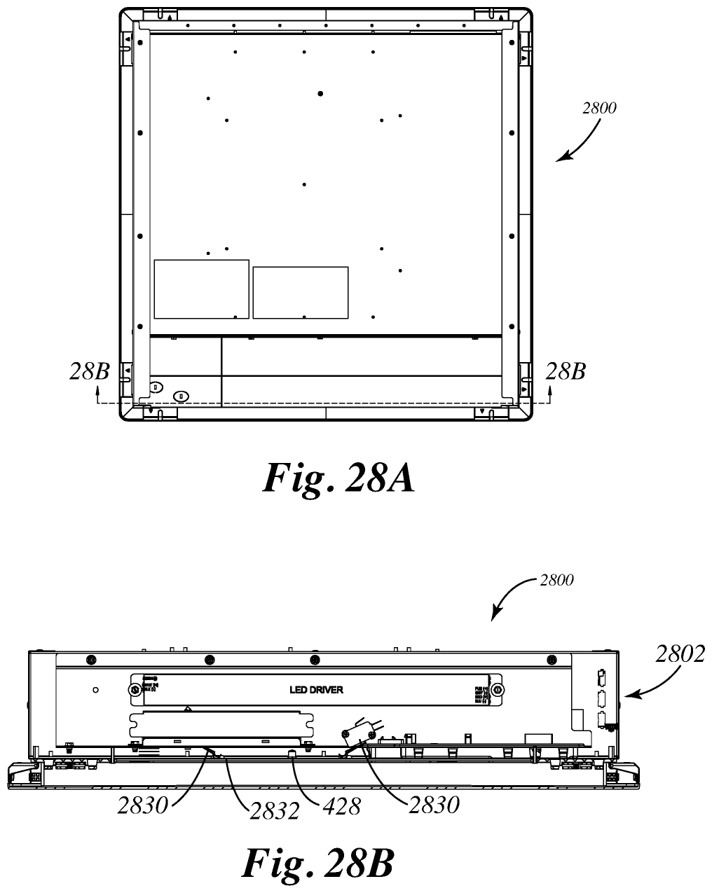

FIG. 28A is a top view of an air treatment ceiling system 2800 according to one embodiment and FIG. 28B is a sectional view of the air treatment ceiling system 2800 of FIG. 28A along the line 28B. A pair of disconnect switches 2830 are shown. The disconnect switches 2830 may alternately be referred to as kill switches. The pair of disconnect switches 2830 provide redundancy such that the air treatment ceiling system 2800 retains the functionality of the disconnect switch 2830 even if one of the disconnect switches 2830 fails. In an alternate embodiment, any suitable number of disconnect switches 2830 may be included in the air treatment ceiling system 2800. In yet another embodiment, the air treatment ceiling system 2800 may not include any disconnect switches 2830.

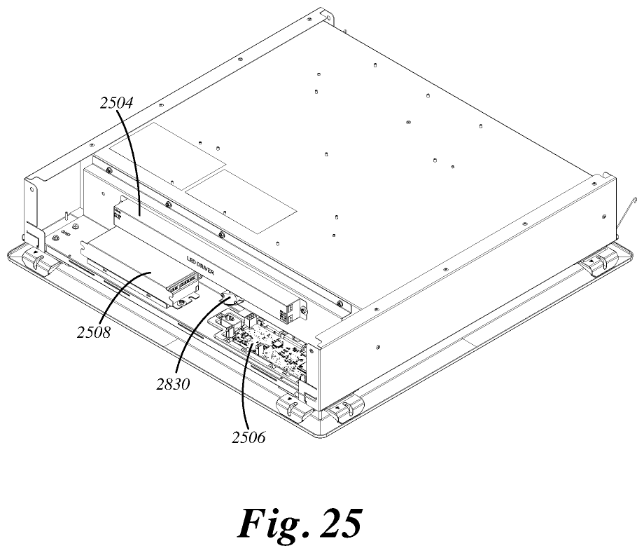

FIG. 25 illustrates a top perspective view of an air treatment ceiling system with an electronic cover panel removed according to one embodiment. FIGS. 28A-B show respective top and sectional views of the same. Some of the system components are shown, including UV ballast 2508 that drive the UV source (not shown), PCB 2506 that includes memory, one or more processors, and other control system components, disconnect or kill switches 2830, and a LED driver to control the downlight.

Referring to FIG. 28A-B, the disconnect switches 2830 are housed in the air treatment body 2802 of the air treatment ceiling system 2800. Each disconnect switch 2830 may be connected to the power source for the UV light. The UV reactor chamber access door 428 can include a pair of disconnect switch contacts 430 (See FIG. 4A) that connect with contacts 2832 that extend from the housing of the respective disconnect switches 2830 when the chamber access door 428 is in the seated position as shown in FIG. 28B. The disconnect switches can be connected to cut the flow of power the UV light (and, if desired, other components) if one or more of the contacts 430 stop making contact with the disconnection switch (such as when the UV chamber access door is opened). As depicted, the disconnect switch 2830 can include multiple disconnect switch contacts 2832 to provide redundancy in case one of the disconnect switch contacts 2832 becomes bent or otherwise fails.

That is, the disconnect switches 2830 can be configured to disconnect power when the UV chamber access door is unseated. Put another way, the disconnect switch contacts 2832 may be configured to disconnect from the access door contacts 430 when the door is not securely latched (e.g., with the thumbscrew) to the air treatment body 2802 of the air treatment ceiling system 2800. When the disconnect switch contacts 2832 are both connected to their respective access door contacts 430, the disconnect switches 2830 make a completed circuit and allow power to flow to the UV light (and other components). When the disconnect switches 2830 are not connected, there is an open circuit that prevents power from flowing to the UV light. In other words, when the chamber access door 428 is not securely latched to the air treatment body 2802 of the air treatment ceiling system 2800, the disconnect switches 2830 disrupt the power source for the UV light and prevent the UV light from being powered and emitting UV light. In one embodiment, the air treatment ceiling system 2800 may send an error message to a remote device when the disconnect switch 2830 indicates that the chamber access door 428 is in an unseated position. In one embodiment, the disconnect switches 2830 can be integrated into a latch so that when the latch becomes disengaged, the contacts 430, 2832 are disconnected.

As depicted, the disconnect switch 2830 uses physical contact to make a complete circuit. In an alternate embodiment, the disconnect switch 2830 may be triggered in any other suitable manner. For example, in one embodiment, the disconnect switch 2830 can be triggered by a magnet in the configurable maintenance door 2810.

Returning to FIG. 17, the configurable maintenance door 1710 (also referred to as grille) can include a functional door module 1740. In one embodiment, the functional door module 1740 may be integral with the configurable maintenance door 1710. In another embodiment, the functional door module 1740 can be a separate component couplable to the configurable maintenance door 1710. In one embodiment, the functional door module 1740 may have a reflective back surface to help reflect the UV light around the reactor chamber. The reflective back surface may alternately be referred to as a reflector. In one embodiment, the reflector of the functional door module 1740 may include a visible light reflector operable to reflect visible light received from a visible light source toward an area of the room. In this way, the reflector may be a two-sided reflector operable to reflect UV light within the reactor chamber and to reflect visible light toward the room.

As depicted in FIG. 17, the functional door module 1740 includes a visible light module 1742 for providing visible light into the room. The visible light module may include a plurality of LEDs and an LED driver circuit operable to supply power to the plurality of LEDs for generating visible light sufficient for illuminating the room area. In one embodiment, the visible light module 1742 can be an LED downlight. In one embodiment, the functional door module 1740 may be a blank panel. In one embodiment, the visible light module 1742 may be a UV to visible light downconverter. In one embodiment, a first configurable maintenance door can have one type of functional door module 1740 and a second configurable maintenance door can have a different functional door module 1740. For example, the first configurable maintenance door may have a visible light module 1742 and the second configurable maintenance door may have a blank. In an alternate embodiment, the first configurable maintenance door and the second configurable maintenance door may have the same type of functional door module 1740. There can be a plurality of configurable maintenance doors 1710 that can each be used as part of the air treatment ceiling system 1700. The configurable maintenance door 1710 may be changed while the air treatment ceiling system 1700 is installed in the ceiling T-rail system.

In one embodiment, the visible light module 1742 may include a visible light source disposed to direct light in a generally transverse manner relative to a target direction of visible light for the visible light module 1742. In one embodiment, the visible light module 1742 can be a side-lit LED module. The visible light source may be disposed within a channel of a frame assembly of the functional door module 1740. The visible light source in one embodiment may be a strip, with a plurality of light sources, that is disposed to engage a base surface of the channel and within the channel along a length of the frame assembly. The visible light source may be captured within the channel by a first and second protrusion spaced away from the base surface of the channel.

A visible light director may be disposed at least partially within the channel. The channel of the frame assembly may support the visible light director such that a portion of a room facing surface of the visible light director is exposed to the room to facilitate directing visible light into the room. The visible light director may include a side surface (e.g., a perimeter surface) operable to receive light from the visible light source. In one embodiment, light received via the side surface may be directed within the visible light director and transverse relative to the side surface toward the room facing surface of the reflector.

In one embodiment, the visible light director may be a lenticular lens operable to facilitate directing light received from the visible light source within the channel toward the room facing surface of the reflector and into the room. The lenticular lens may include one or more physical aspects (e.g., holes or depressions) that facilitate directing light from within the lenticular lens to an external area. The lenticular lens may be disposed proximal to the reflector, and may receive light from one or more light sources, which may be disposed at one or more sides of the lenticular lens.

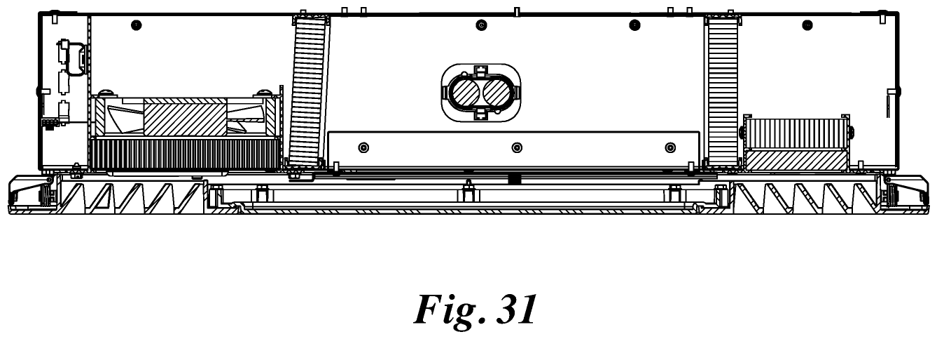

In alternative configurable maintenance doors, the functional door module does not include a light at all. The bottom surface of the UV air treatment system can have a clean and simple aesthetic that matches other ceiling tiles. FIG. 31 illustrates a sectional view of an air treatment ceiling system without a visible lighting element in the configurable maintenance door.

As shown in FIG. 4 and FIG. 30C, the configurable maintenance door 410, 3010 or bottom of the UV air treatment base 402, 3002 can include a gasket 480 or other sealing protrusion. When the grille 410, 3010 is installed to the UV air treatment base 402, 3002, the gasket 480 between them can seal plenum airflow from reaching the UV air treatment system intake 3014.

FIG. 4A shows a top view and FIG. 4C a bottom perspective view of an air treatment ceiling system 400 without a configurable maintenance door installed and with the UV reactor chamber access door 428 in the open position according to one aspect. The UV source 412 is easily accessible and replaceable with the reactor chamber access door 428 in this position. The air treatment ceiling system 400 can include a gasket (not shown) surrounding the reactor chamber opening 426 such that airflow and light from the UV reactor chamber 414 are sealed from entering or exiting the chamber access door 428 when the UV reactor chamber access door 428 is in the closed position. The gasket 427 may create a seal between the UV reactor chamber access door 428 and the area surrounding the reactor chamber opening 426 when the UV reactor chamber access door is in the closed/seated position. As shown in FIG. 4A, the bottom surface 400 of the UV air treatment body 1702 can include an electronics housing 404. The electronics housing 404 can be used to house the electronics for the air treatment ceiling system 400. In this embodiment, the inlet chamber, the reactor chamber 414, and the outlet chamber have a length that is less than the length L1 of the air treatment ceiling system 400.

Returning to FIG. 17, The functional door module 1740 can be interchanged between various modules, such as a UV light converter, a visible light, and an aesthetic panel to name a few examples. The UV light converter may be a UV light downconverter operable to convert UV light from the reactor chamber to visible light. In such an embodiment, the UV light would be routed from the UV reactor chamber to the converter. The UV light converter may include a substrate (e.g., glass) on which a film is disposed, where the film is operable to convert UV light to visible light. The film may be a down conversion layer, and the substrate may be light transmissive. The film may be disposed upstream of the substrate relative to the UV light source so that UV light from the UV light source may be converted to visible light before traveling through the substrate and into the room area.

The UV light converter may constructed in a variety of ways, including downconverting nanophosphors, which may be formed of SiO2 co-doped with Ce and Tb, or nano-crystal with different band gaps to provide down conversion. These structures may be provided on or form the film to enable down conversion of the UV light output from the UV light source to visible light.

The UV light converter in accordance with one embodiment may provide a passive converter or passive conversion system for converting UV light to visible light. The air treatment ceiling system 1700 may not utilize power 1) to convert the UV light or 2) to generate visible light separately from the UV light source, or both.

The UV light converter may be configurable in a variety of ways, depending on the application. In one embodiment, the UV light converter may be configurable to customize the configurable maintenance door 1710 without substantial modification to the configurable maintenance door 1710. For instance, the UV light converter may be configurable for a target color temperature, based on user selection or parameters. The UV light converter may be configurable for such a target color temperature without affecting the overall build of the configurable maintenance door 1710, enabling the configurable maintenance door to be manufactured for applications regardless of the target color temperature. As an example, the UV light converter is replaceable with another UV light converter capable of providing visible light having a second color temperature different from a first color temperature of visible light that is output from the UV light converter. One or more additional or alternative parameters may be affected by the UV light converter, enabling the configurable maintenance door 1710 to be manufactured for applications regardless of the additional or alternative parameters.

The UV light converter, in one embodiment, may be replaceable in the field after the configurable maintenance door 1710 has been installed to vary one or more characteristics of the configurable maintenance door 1710. In one embodiment, the configurable maintenance door 1710 may be replaced after the air treatment ceiling system 1700 has been installed to change the UV light converter in the functional door module 1740.

In one embodiment, the functional door module 1740 may include a visible light regulator operable to control emission of visible light into the room. The visible light regulator may be operable to selectively control emission of visible light into the room area based on directive from the control system. As an example, the visible light regulator may include one or more apertures selectively transmissive with respect to visible light output from the UV light converter.

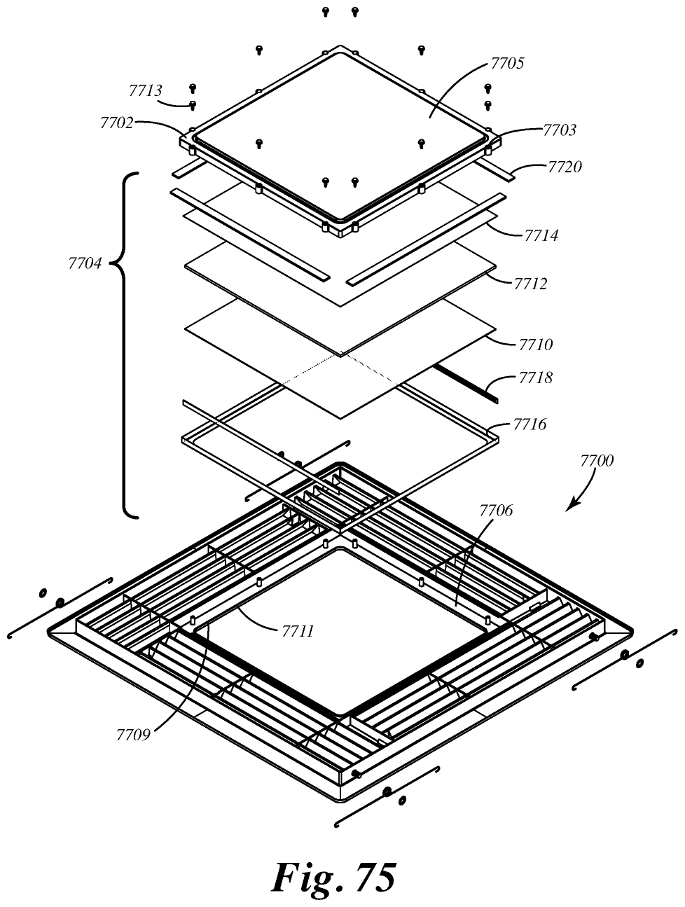

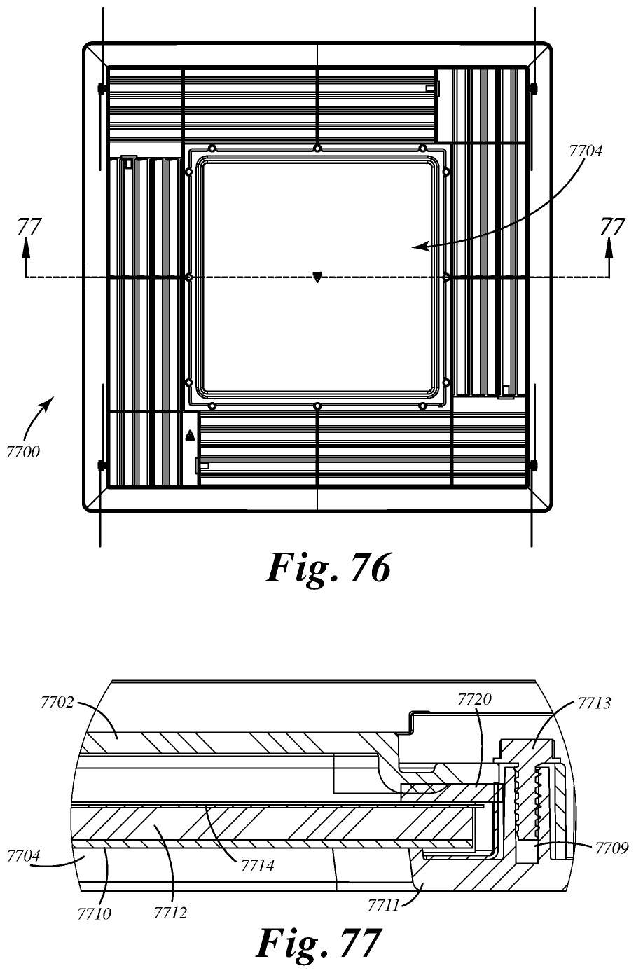

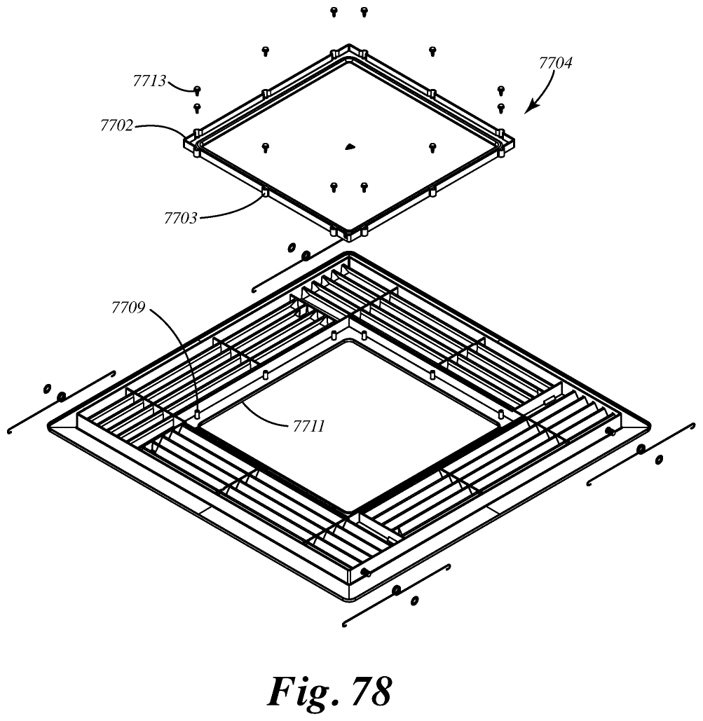

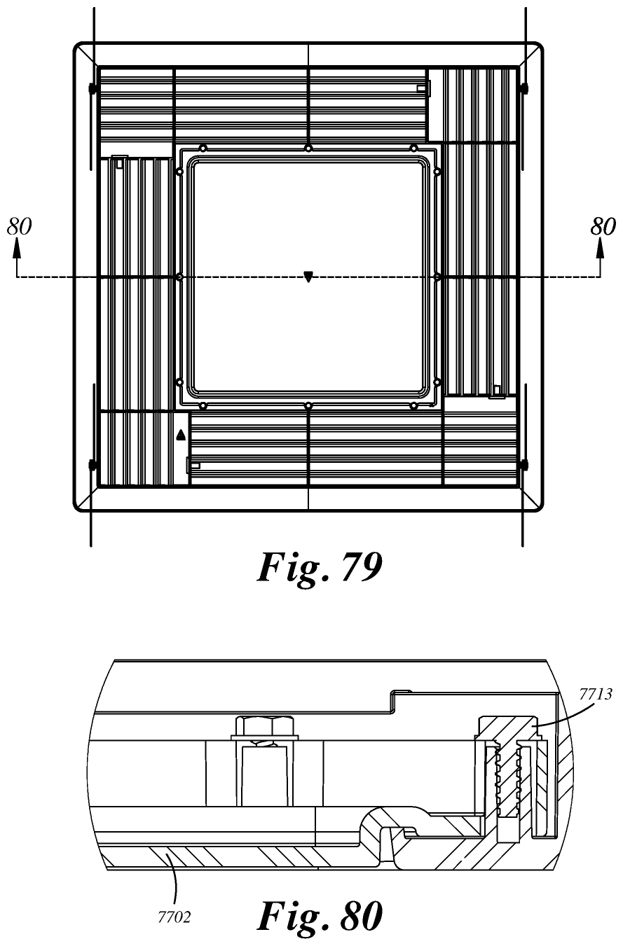

One exemplary visible light functional door module 1740 for a configurable maintenance door is illustrated in FIGS. 75-77. FIG. 75 illustrates a perspective view, FIG. 76 a bottom view, and FIG. 77 a partial sectional view cut along line 77 of FIG. 76. The depicted functional door module 1740 includes a visible light module 7704 mounted between the reversible grille cover 7702 and the grille base 7706. Perhaps as best shown in the perspective view, the visible light module 7704 can include a diffuser 7710, light guide panel 7712, reflector 7714 and a light source (e.g., LED 7716) to side light the light guide panel. The reversible grille cover 7714 can be secured to the grille base (also referred to as the configurable maintenance door) with the visible light module 7704 secured therebetween. FIGS. 76-81 depict a blank functional door module where the reversible cover 7714 has been reversed and its outer surface has been exposed.

In an alternative embodiment, the UV light converter may be an up converter that is configured to convert visible light to UV light. In one embodiment, the functional door module 1740 may include a visible light source capable of generating visible light for illuminating the room area. The visible light from the visible light source may be directed toward the UV light converter and toward the UV reactor chamber. The UV reactor chamber may alternately be referred to as a UV treatment chamber or reactor chamber. The UV light converter may up convert the visible light to UV light for disinfection of air flowing through the reactor chamber. Example configurations of an up conversion configuration may include lanthanide-doped upconversion phosphor (UCP) materials, such as lanthanide-doped upconversion luminescent nano- and microcrystalline Y₂SiO₅.

The configurable maintenance door 1710 includes an inlet 1750. Returning to FIG. 30C, the inlet 3012 can include at least one inlet opening 3014. The inlet 3012 is configured to permit airflow into the inlet chamber 3050 through the inlet chamber opening 3056. In one embodiment, the inlet 3012 may be curved so the inlet opening 3014 is offset from the inlet collimator 3040. In one embodiment, the inlet 3012 may be curved so the inlet opening 3014 is offset from the filter 3080. The curvature can help direct airflow through the inlet 3012 into the inlet chamber 3050.

Optionally, the inlet 3012 may include at least one inlet louver 3013 defining the inlet opening 3016. The inlet louver 3013 may alternately be referred to as an inlet vent. The inlet louver 3013 can be configured to direct the airflow into the inlet chamber 3050. The at least one inlet louver 3013 can have a variety of louver orientations. FIGS. 18A-D show configurable maintenance doors 1810 according to various embodiments. Each configurable maintenance door 1810 in FIGS. 18A-D includes an inlet 1812, an outlet 1816, and a functional door module 1840. FIGS. 18A-D show a variety of configurations of the inlet 1812 and outlet 1816, some including one or more louvers 1813. The louvers may be a plurality of apertures arranged in multiple formations. In one embodiment, the apertures may be round. In another embodiment, the apertures can be hexagonal. In yet another embodiment, the apertures may be any other suitable shape. The louvers may be a plurality of apertures arranged in one formation. In some embodiments, the louvers may be a plurality of slats arranged in one or multiple formations, such as depicted in FIG. 18A. The various grille configurations permit airflow to the inlet and from the outlet of the UV air treatment body above. The inlets and outlets of the grille/configurable maintenance door need not align precisely or at all with the inlets and outlets of the UV air treatment body above. In this way, the louvers can be designed symmetrically or in other aesthetically pleasing patterns without regard to the inlet and outlet placement in the UV air treatment body above. While certain louver patterns are depicted in FIGS. 18A-D, it should be noted that the louvers can essentially have any louver pattern suitable for a particular application. FIGS. 19A-D show configurable maintenance doors 1810 according to various embodiments. The configurations shown in FIGS. 19A-D are the same as those shown in FIGS. 18A-D but with a different functional door module 1940 installed.

As shown in FIG. 17, the configurable maintenance door 1710 includes an outlet 1760. Returning to FIG. 30C, the outlet 3016 can include at least one outlet opening 3018. The outlet 3016 is configured to permit airflow out of the outlet chamber 3030 through the outlet chamber opening 3036. The fan assembly 3070 can pull air into the air treatment ceiling system 3000 through the at least one inlet opening 3014 and push air out of the air treatment ceiling system 3000 through the at least one outlet opening 3018. In one embodiment, the outlet 3016 may be curved so the outlet opening 3018 is offset from the outlet collimator 3040. The curvature can help direct airflow out of the outlet chamber 3030 through the outlet 3016.

The outlet 3016 may include at least one outlet louver 3017 defining the outlet opening 3018. The outlet louver 3017 may alternately be referred to as an outlet vent. The outlet louver 3017 can be configured to direct the airflow out of the outlet chamber 3030. The at least one outlet louver 3017 may have any suitable louver orientation, such as those described above with reference to FIGS. 18A-J and inlet louver 1813. In one embodiment, the outlet louver 3017 can have the same louver orientation as the inlet louver 3013. In another embodiment, the outlet louver 3017 can have a different louver orientation from the inlet louver 3013. The inlet louver 3013 and the outlet louver 3017 may be configured to absorb UV light emitted from the germicidal light source 3060. Put another way, the inlet louver 3013 and the outlet louver 3016 may interact positively with the UV light. The inlet louver 3013 and the outlet louver 3016 can provide one or more additional reflection for the UV light emitted from the germicidal light source 3060 to reduce the amount of UV light escaping the air treatment ceiling system 3000.

The inlet louver 3013 and the outlet louver 3016 provide a pathway for air into and out of the air treatment ceiling system 3000 respectively through the configurable maintenance door 3010. The inlet louver 3013 and the outlet louver 3016 can be configured to reduce the amount of UV light escaping the air treatment ceiling system 3000 with minimal restriction of airflow into and out of the air treatment ceiling system 3000. In one embodiment, the inlet louver 3013 may be configured at an angle relative to the inlet chamber opening 3056 and the outlet louver 3016 may be configured at an angle relative to the outlet chamber opening 3036. As depicted in FIG. 30C, the outlet louver 3016 can be configured at an angle relative to the collimator 3040 at the outlet chamber opening 3036. As depicted in FIGS. 30C and 30D, the inlet louver 3013 may be configured to align with the air path 3082 through the air filter 3080 but at an angle relative to the collimator 3040 nearest the inlet chamber opening 3056. In an alternate embodiment, the inlet louver 3013 can be configured at an angle relative to the air path 3082 through the air filter 3080.

There may be multiple configurable maintenance doors 3010 for use with the air treatment ceiling system 3000. Having multiple configurable maintenance doors 3010 can enable a wide array of shapes, colors, styles, and configurations of the air treatment ceiling system 3000. In one embodiment, each configurable maintenance door 3010 can have a distinct configuration of the louver orientation of the inlet louver 3013 and the outlet louver 3016. In an alternate embodiment, two or more configurable maintenance doors 3010 may have the same louver orientation for both the inlet louver 3013 and the outlet louver 3016.

In another embodiment of the system is a design that enables easy snap details for in ceiling installation or a suspended t-rail ceiling structure.

### Ceiling Integration System

The air treatment ceiling system as described above is configured to fit within a grid opening of a ceiling T-rail system. However, ceiling T-rail systems (and correspondingly grid openings) come in a variety of sizes based on common suspended ceiling standards in both standard and metric based buildings. Put another way, a U.S. ceiling T-rail system generally has different measurements from a metric ceiling T-rail system. It is desirable to have one size of air treatment ceiling system that can be used across a variety of ceiling T-rail configurations. This can result in an unsightly gap between the air treatment ceiling system and the ceiling T-rail system. Ceiling T-rail systems have limited plenum space for installation and housing of an air treatment ceiling system. Therefore, it is desirable to install the air treatment ceiling system substantially vertically. This may be referred to as z-axis installation. A ceiling integration system can be used to install an air treatment ceiling system in a ceiling T-rail system as described above. A ceiling integration system may be used to create an air treatment ceiling system with an invisible recessed profile with respect to the ceiling T-rail system.

FIGS. 14A-D show one embodiment of a UV air treatment body 1410 installed on four different ceiling T-rail systems 1440. FIGS. 16A-D are each a cross-section of the air treatment ceiling system of FIGS. 14A-D. FIGS. 14A-D and 16A-D illustrate the need for a ceiling integration system that can be used with different ceiling T-rail systems 1440. In one embodiment, the ceiling T-rail system 1440 of FIG. 14A can have an outer width WO of 24 inches, an inner width WI of 23.063 inches, and a T-rail thickness T of 15/16 inch resulting in a first ceiling T-rail gap G1 of 0.688 inches and a second ceiling T-rail gap G2 of 0.178 inches. In one embodiment, the ceiling T-rail system 1442 of FIG. 14B can have an outer width WO of 600 millimeters, an inner width WI of 22.677 inches, and a T-rail thickness T of 24 millimeters resulting in a first ceiling T-rail gap G1 of 0.496 inches and a second ceiling T-rail gap G2 of 0.014 inches. In one embodiment, the ceiling T-rail system 1444 of FIG. 14C can have an outer width WO of 24 inches, an inner width WI of 23.438 inches, and a T-rail thickness T of 9/16 inch resulting in a first ceiling T-rail gap G1 of 0.876 inches and a second ceiling T-rail gap G2 of 0.366 inches. In one embodiment, the ceiling T-rail system 1446 of FIG. 14D can have an outer width WO of 600 millimeters, an inner width WI of 23.071 inches, and a T-rail thickness T of 14 millimeters resulting in a first ceiling T-rail gap G1 of 0.692 inches and a second ceiling T-rail gap G2 of 0.182 inches.

In order to provide proper seating on the different t-rail systems 1440, 1442, 1444, 1446 a different size vertical installation clip can be utilized in the vertical installation clip assembly. That is, depending upon which ceiling grid system the UV air treatment system will be installed, a different size vertical installation clip can be provided without changing anything else about the UV air treatment fixture. The grille 1710 will cover all of the different size gaps and provide proper sealing between the plenum and the UV air treatment body regardless of which size clips are utilized in the vertical clip assemblies.

FIGS. 16A-D illustrate sectional views of the same UV air treatment bodies 1410, 1412, 1424, 1426 except the grilles 1420, 1422, 1424, 1426 are installed to show how the grilles conceal the gaps between the t-rail and the UV air treatment bodies. From these sectional views, it is clear how the sides of the grille butt up against the t-rail. The gaps are covered regardless of the specifications of the t-rail, though as can be seen the grille face extends over the bottom of the t-rail less or more depending on the size of the t-rail. In FIG. 16A the gap has the largest span while in FIG. 16D the gap has the smallest span. The seals 1450, 1452, 1454, 1456 between the grilles 1420, 1422, 1424, 1426 and the respective UV air treatment bodies 1410, 1412, 1414, 1416 preventing airflow from the plenum to the UV air treatment inlet can be seen in FIGS. 16A-D.

FIGS. 9A-9B show a ceiling integration system 900 for an air treatment ceiling system 910 according to one embodiment. The ceiling integration system 900 can include a deployable mounting system 920 and a trim system 930. The deployable mounting system 920 can be configured to support the air treatment ceiling system 910 in a grid opening 942 of the ceiling T-rail system 940. The deployable mounting system 910 can deploy to contact the ceiling T-rail system 940. As depicted, the deployable mounting system includes a plurality of flexible support members 922 configured to flex toward the air treatment ceiling system 910 during installation and to extend away from the air treatment ceiling system 910 to contact the ceiling T-rail system 940. In one embodiment, the flexible support members 922 can flex toward the air treatment ceiling system 910 through manual application of force toward the air treatment ceiling system 910. The flexible support members 922 can extend away from the air treatment ceiling system 910 when the force is released. As depicted, the flexible support members 920 each include a foot 924. The foot 924 can rest against the ceiling T-rail system 940 when the flexible support members are deployed. In one embodiment, the foot 924 may rest against a T-rail corner 944. In one embodiment, the foot 924 may rest against a mounting surface 946 of the T-rail system 940. The flexible support members 922 may be sized to support the air treatment ceiling system in a variety of sizes of the grid opening 942. For example, in a larger grid opening 942, the flexible support members 922 may be fully expanded and in a smaller grid opening 942, the flexible support members may be compressed toward the air treatment ceiling system 910. The deployable mounting system 920 can center the air treatment ceiling system 910 in the grid opening 924 to give the air treatment ceiling system 910 a uniform appearance.

As depicted, the deployable mounting system 920 includes three flexible support members 922 on each of the longer sides of the air treatment ceiling system 910 and one flexible support member 922 on each of the shorter sides of the air treatment ceiling system 910. In an alternate embodiment, any other suitable number of flexible support members 922 may be used. In one embodiment, each side of the air treatment ceiling system 910 can incorporate a different number of flexible support members 922. In one embodiment, the deployable mounting system 920 can be incorporated on two sides of the air treatment ceiling system 910. In another embodiment, the deployable mounting system 920 may be incorporated on one side of the air treatment ceiling system 910 or on three sides of the air treatment ceiling system 910.

In one embodiment, the flexible support members 922 can be locking clips or spring clips. The flexible support members 922 may be held in place on the air treatment ceiling system 910 with two slots in the air treatment ceiling system 910. In an alternate embodiment, the flexible support members 922 may be attached to the air treatment ceiling system 910 using common riveting or fasteners, or any other suitable means. In one embodiment, one slot may be located near the bottom edge of the air treatment ceiling system 910 and the other slot may be located upward from the first slot. The outer portion of the flexible support members 922 can be a spring clip detail that allows the outer portion to be retracted. During installation the spring clip can be pressed into place (toward the air treatment ceiling system 910) and the spring may lock between the two slots held by the spring material. The outer portion can spring tension the air treatment ceiling system 910 to the ceiling T-rail system 940.

Once the deployable mounting system 920 is deployed, a ceiling T-rail gap 948 is created between the air treatment ceiling system 910 and the ceiling T-rail system 940. The ceiling T-rail gap 948 may be along one side, two sides, three sides, or all four sides of the air treatment ceiling system 910. The trim system 930 may be configured to conceal the deployable mounting system 920 and the ceiling T-rail gap 948. As depicted in FIG. 9B, the trim system 930 can include a concealing element 932 and a coupling element 934 extending perpendicularly from a back surface of the concealing element 932. In an alternate embodiment, the coupling element 934 can be joined to the back surface of the concealing element 932 in any suitable orientation and by any suitable means. In one embodiment, the coupling element 934 may be integrated into the concealing element 932. The coupling element 934 may be configured to couple the concealing element 934 to the air treatment ceiling system 910. In the depicted embodiment, the coupling element 934 couples to the air treatment ceiling system 910 through an opening in the front or room-facing surface of the air treatment ceiling system. As depicted, the coupling element 934 is a Christmas tree fastener that is coupled to the air treatment ceiling system 910 through the application of force. In alternate embodiments, the coupling element 934 may couple to the air treatment ceiling system 910 through any suitable coupling mechanism. For example, in one embodiment, the coupling element 934 can be a magnet that is coupled to the air treatment ceiling system 910 through magnetic force.

The trim system 930 can be a variety of sizes to account for different sizes of the ceiling T-rail gap 948 and differing dimensions of the air treatment ceiling system 910. For example, in one application, the trim system 930 may include two sizes of concealing elements 932 in sets of two. One size of the concealing elements 932 may be used on the length of the air treatment ceiling system 910 and the other size of concealing elements 932 may be used on the width of the air treatment ceiling system 910. A given size of the trim system 930 can be used for different sizes of the ceiling T-rail gap 948 by including multiple coupling points for the coupling element 934 in the air treatment ceiling system 910. For example, the coupling element 934 can couple to a coupling point closer to the center of the air treatment ceiling system 910 to cover a smaller ceiling T-rail gap 948 and can couple to a coupling point closer to the outer edge of the air treatment ceiling system 910 to cover a larger ceiling T-rail gap 948. In one embodiment, the ceiling integration system 900 may be made from UL approved flame resistant plastic.

Various configurations of the deployable mounting system and the trim system are discussed throughout the disclosure. It will be noted that the configurations may be used independently or in combination with another deployable mounting system configuration or trim system configuration. The deployable mounting system and the trim system can be incorporated on one or more sides of the air treatment ceiling system.

The air treatment ceiling system can be installed in a grid opening of a ceiling T-rail system using a ceiling integration system and the following method. The ceiling integration system may be moved to an installation position. In the embodiment of FIGS. 9A-9B, this can include compressing or flexing the flexible support members 922 toward the air treatment ceiling system 910. The air treatment ceiling system can be lifted into a ceiling plenum until at least a portion of the ceiling integration system is above a ceiling T-rail system. The ceiling integration system may be deployed to contact the ceiling T-rail system. In the embodiment of FIGS. 9A-9B, this can include releasing or extending the flexible support members 922 so that they may expand away from the air treatment ceiling system 910 to contact the ceiling T-rail system 940. The air treatment ceiling system may need to be lowered for the ceiling integration system to contact the ceiling T-rail system. The trim system of the ceiling T-rail system can be deployed to cover the ceiling T-rail gap. In the embodiment of FIGS. 9A-9B, this can include coupling the trim system 930 to the air treatment ceiling system 910 through the coupling element 934 to cover the ceiling T-rail gap 948.

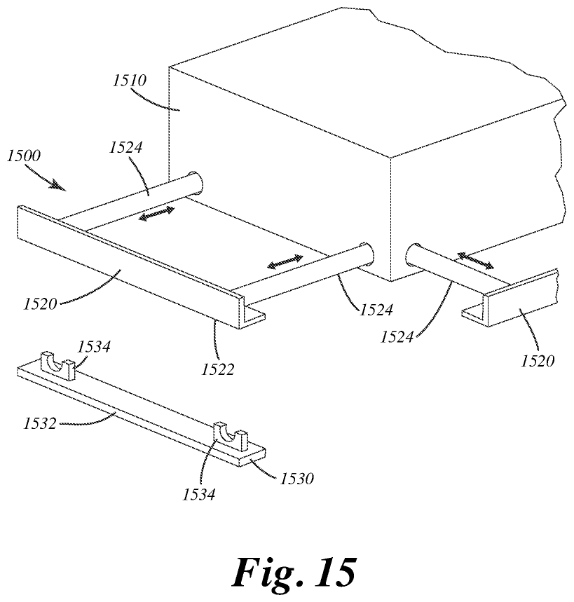

FIG. 15 shows a ceiling integration system 1500 according to one embodiment. The ceiling integration system 1500 can include a deployable mounting system 1520. The deployable mounting system 1522 can include a support member 1522 and at least one rod 1524 coupled to the support member 1522. The at least one rod 1524 may be moveably coupled to the air treatment ceiling system 1510 to allow the support member 1522 to move toward and away from the air treatment ceiling system 1510. The ceiling integration system 1500 is in an installation position when the support member 1522 is moved toward the air treatment ceiling system 1510. The at least one rod 1524 can be accessed behind the door of the air treatment ceiling system 1510 to allow an installer to move the at least one rod 1524 and consequently move the support member 1522 to contact the ceiling T-rail system. In another embodiment, the at least one rod 1524 may be moveable in any other suitable way. The ceiling integration system 1500 is deployed when the support member 1522 contacts the ceiling T-rail system. The at least one rod 1524 may be moved any suitable length to span the ceiling T-rail gap. The ceiling integration system 1500 can include a trim system 1530. The trim system 1530 may couple to the at least one rod 1524 to conceal the ceiling T-rail gap. This may be referred to as deploying the trim system 1530. The trim system 1530 may include at least one protrusion 1534 extending from a concealing element 1532. The protrusion 1534 can allow the trim system 1530 to attach to the at least one rod 1524.

In some embodiments, the trim system may be integrated into the deployable mounting system. FIG. 7 shows a ceiling integration system 700 according to one embodiment. The ceiling integration system 700 can include an integration element 720 and a coupling element 730. In this configuration, the integration element 720 functions as both the deployable mounting system and the trim system. The ceiling integration system 700 can be coupled to the air treatment ceiling system 710 through an opening in the air treatment ceiling system 710. The coupling element 730 can be coupled to the air treatment ceiling system 710 through the opening. As depicted, the coupling element 730 is a Christmas tree fastener. In one embodiment, the ceiling integration system 700 may be removably attached to the air treatment ceiling system 710 through the coupling element 730. The integration element 720 may rest on the ceiling T-rail system 740 to support the air treatment ceiling system 710 in a grid opening. In one embodiment, the ceiling integration system 700 may be molded.

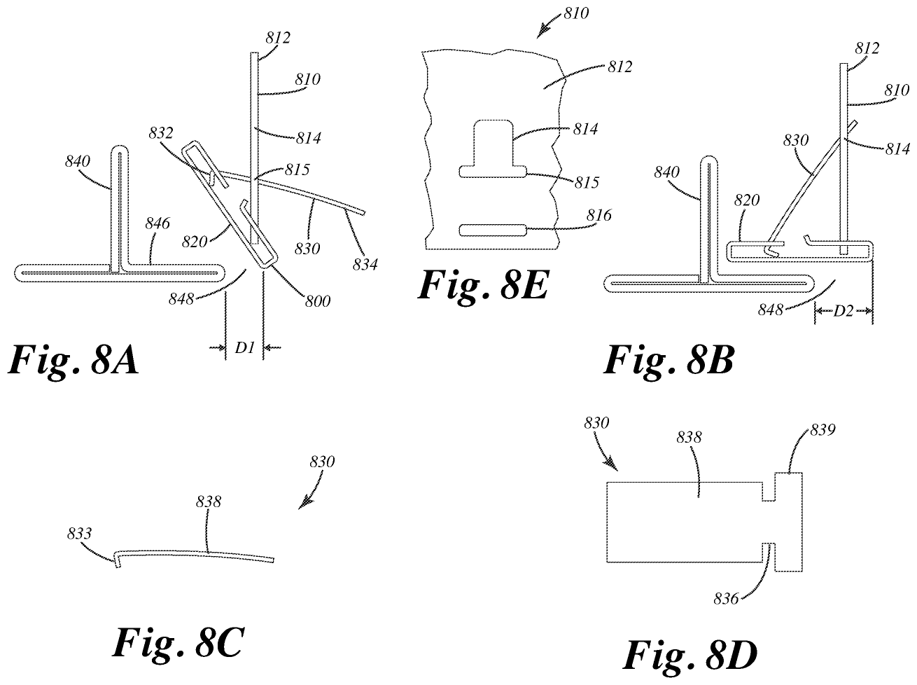

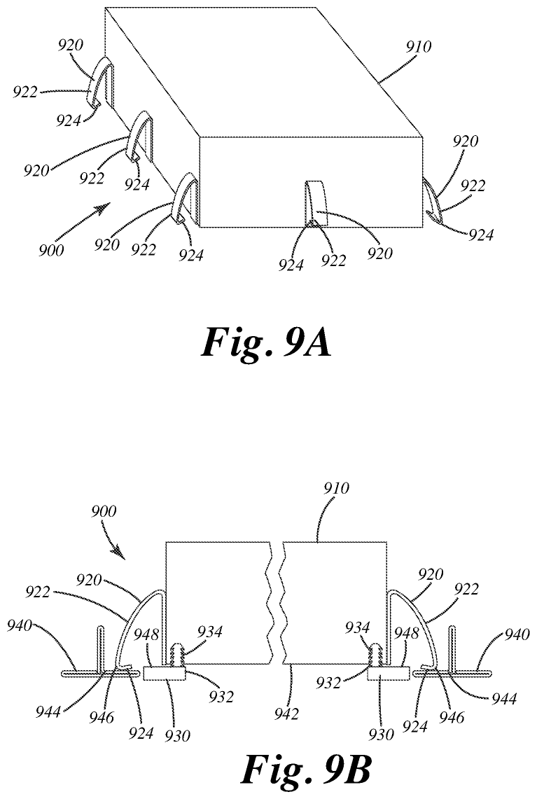

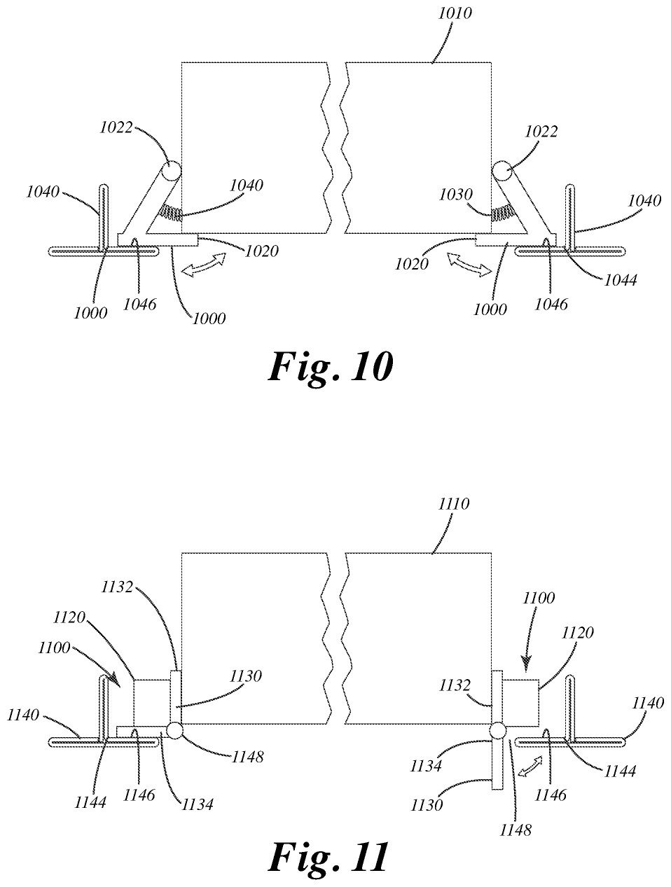

FIGS. 8A-E show a ceiling integration system 800 according to one embodiment. FIG. 8A shows the ceiling integration system 800 in an installation position. The distance D1 may refer to a minimum installation clearance for the trim ring 810. The ceiling integration system 800 can include a collapsible support member 820 and a clip 830. The collapsible support member 820 may be rotatably coupled to the air treatment ceiling system 810. In the installation position, the collapsible support member 820 may be rotated toward the air treatment ceiling system 810 as shown in FIG. 8A. The collapsible support member 820 can be configured to be parallel to a front or room-facing surface of the air treatment ceiling system 810 in a deployed position as shown in FIG. 8B. The collapsible support member 820 can contact the ceiling T-rail system 840 in the deployed position. This may also be referred to as deploying the ceiling integration system 800.

The clip 830 can be coupled to the collapsible support member 820 near a first end 832 of the clip 830. FIG. 8C is a side view of the clip 830 according to one embodiment. The clip 830 may include a locking tab 838 and a locking hook 833. The locking hook 833 can couple the clip 830 to the collapsible support member 820. In one embodiment, the locking tab 838 may be a spring locking tab. The air treatment ceiling system 810 may include a wall 812. The wall 812 can include a clip entry assembly points 814 and trim rail slots 816. The clip 830 may be configured to contact the air treatment ceiling system 810 substantially at a second end 834 of the clip 830 to secure the collapsible support member 820 in the deployed position as shown in FIG. 8B. The clip 830 may be secured to the wall 812 of the air treatment ceiling system 810 at a clip locking point 814. In the deployed position, the collapsible support member 820 can be moved toward and away from the air treatment ceiling system 810 to correspond to the size of the ceiling T-rail gap 848. This movement may also be referred to as deploying the trim system of the ceiling integration system 810. The distance D2 may refer to a US/Metric gap.

The ceiling integration system 800 may be locked in the deployed position. FIG. 8D is a top view of the clip 830 of FIG. 8C. As seen in FIGS. 8C-E, the clip 830 may include a spring locking tab 838. From the installation position, the spring locking tab 838 can be pressed to bring the collapsible support member 820 parallel with the mounting surface 846 of the ceiling T-rail system 840. The spring locking tab 838 may flex just before it reaches the deployed position shown in FIG. 8B allowing the locking notch 836 to move into the smaller passage of the clip entry detail locking the tab 838 (and thus the clip 830) in place. A retention protrusion 839 may contact an inner surface of the wall 812 of the air treatment ceiling system 810 to retain the clip 830 in the locked position.

FIG. 10 shows a ceiling integration system 1000 according to one embodiment. The ceiling integration system 1000 may provide a reduced footprint while the air treatment ceiling system 1010 is being installed. The ceiling integration system 1000 may include a support member 1020 and a hinge 1022. The hinge 1022 can be coupled to the air treatment ceiling system 1010. The support member 1020 may be rotatably coupled to the air treatment ceiling system 1010 through the hinge 1022. A compressible material 1030 may be coupled to the support member 1020 at one end and the air treatment ceiling system 1010 at a second end. In one embodiment, the compressible material 1030 is a spring. The compressible material 1030 may be configured to bias the support member 1020 away from the air treatment ceiling system 1010. Put another way, the compressible material 1030 can tension the support member 1020 outward to the maximum dimensional position.

The support member 1020 can be compressed toward the air treatment ceiling system 1010 during installation of the air treatment ceiling system 1010. This may be referred to as moving the ceiling integration system 1000 to the installation position. The support member 1020 may be released to contact the ceiling T-rail system 1040. This may be referred to as deploying the ceiling integration system 1000. When the support member 1020 is released, the compressible material 1030 can bias the support member 1020 away from the air treatment ceiling system 1010. This may be referred to as deploying the trim system to cover the ceiling T-rail gap. In some ceiling T-rail systems 1040, the support member 1020 may contact a T-rail corner 1044. The force of contact between the support member 1020 and the T-rail corner 1044 can compress the compressible material 1040 and move the support member 1020 toward the air treatment ceiling system 1010. This is one way the ceiling integration system 1000 can be used for multiple ceiling T-rail system configurations. If the support member 1020 does not contact the T-rail corner 1044, the support member 1020 can rest on the mounting surface 1046.

FIG. 11 shows a ceiling integration system 1100 according to one embodiment. The ceiling integration system 1100 can include a plano hinge 1130 and a stop 1120. The stop 1120 may alternately be referred to as a one way stop or a tab. The plano hinge 1130 can be coupled to the air treatment ceiling system 1110 on a first surface of a first side 1132 of the plano hinge 1130. The stop 1120 can be coupled to the plano hinge 1130 on a second surface of the first side 1132 of the plano hinge 1130. In one embodiment, the stop 1120 may be bent out of sheet metal. The plano hinge 1130 may be extended as shown on the right side of FIG. 11 during installation of the air treatment ceiling system 1110. This may be referred to as moving the ceiling integration system 1100 to an installation position. The plano hinge 1130 can be rotated to contact the stop 1120 on a second surface of a second side 1134 of the plano hinge 1130 when the air treatment ceiling system 1110 is lifted above the ceiling T-rail system 1140. Put another way, the plano hinge 1130 may be bent to a 270° angle. This may be referred to as deploying the trim system of the ceiling integration system 1100. The stop 1120 may prevent the plano hinge 1130 from rotating upward in order to secure the air treatment ceiling system 1110 to the ceiling T-rail system 1140. A first surface of the second side 1134 of the plano hinge 1130 may contact the ceiling T-rail system 1140. This may be referred to as deploying the ceiling integration system 1100.

When the ceiling T-rail gap 1148 is smaller, the plano hinge 1130 may contact the T-rail corner 1144. When the ceiling T-rail gap 1148 is bigger, the plano hinge 1130 can rest on a mounting surface 1146 of the ceiling T-rail system 1140. The plano hinge 1130 is held in place against the stop 1120 in the deployed position through the force of gravity. To remove the air treatment ceiling system 1110 from the ceiling T-rail system 1140, the air treatment ceiling system 1110 can be lifted until the hinge 1130 is vertical, which allows the air treatment ceiling system 1110 to be removed through the grid opening.

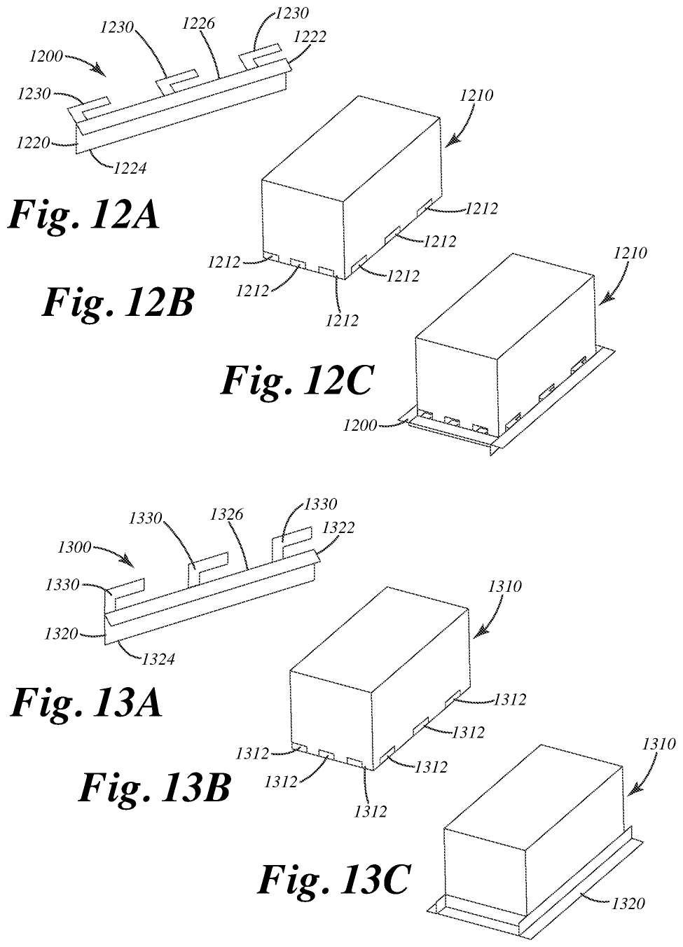

FIGS. 12A-C show a ceiling integration system 1200 according to one embodiment. As seen in FIG. 12A, the ceiling integration system 1200 includes a perpendicular support member 1220. The perpendicular support member 1220 having a first support member 1222, a second support member 1224, and a seam 1226 joining the first support member 1222 and the second support member 1224. In one embodiment, the first support member 1222 and the second support member 1224 are the same size. In another embodiment, the first support member 1222 is a different size than the second support member 1224. A coupling member 1230 can extend from the seam 1226. As depicted, the coupling member 1230 extends from the seam 1226 parallel to the first support member 1222. As depicted, the ceiling integration system 1200 includes three coupling members 1230. In an alternate embodiment, any suitable number of coupling members 1230 may be used. In an alternate embodiment, coupling member 1230 can have a different configuration from what is depicted in FIG. 12A. As seen in FIG. 12B, the air treatment ceiling system 1210 can include a plurality of apertures 1212 configured to align with the coupling members 1230. The coupling member 1230 may be configured to couple the perpendicular support member 1220 to the air treatment ceiling system 1210 through the apertures 1212. This may be referred to as deploying the trim system of the ceiling integration system 1200. As depicted, the coupling member 1230 can be inserted into an aperture 1212 and moved to contact a wall of the aperture 1212. In an alternate embodiment, the coupling member 1230 can be coupled to the air treatment ceiling system 1210 through any suitable means. In one embodiment, the air treatment ceiling system 1210 may first be lifted above the ceiling T-rail system and then the ceiling integration system 1200 may be coupled to the air treatment ceiling system 1210. The first support member 1222 can contact the ceiling T-rail system and support the air treatment ceiling system 1210 in the grid opening. This may be referred to as deploying the ceiling integration system 1200.

In one embodiment, the ceiling integration system 1200 may include two sets of two configurations of the perpendicular support member 1220. In one embodiment, the ceiling integration system 1200 can include two longer perpendicular support members 1220 and two shorter perpendicular support members 1220. In one embodiment, one set of perpendicular support members 1220 may have a first support member 1222 that extends beyond the coupling member 1230. In another embodiment, the ceiling integration system 1200 can have any suitable configuration of perpendicular support members 1220.

FIGS. 13A-C show a ceiling integration system 1300 according to one embodiment. The ceiling integration system 1300 is similar to the ceiling integration system 1200 shown in FIGS. 12A-C except as otherwise noted. The coupling member 1330 may extend from the seam 1326 parallel to the second support member 1324. When coupled to the air treatment ceiling system 1310, the first support member 1322 contacts the air treatment ceiling system 1310. The second support member 1324 may contact the ceiling T-rail system and support the air treatment ceiling system 1310. This may be referred to as deploying the ceiling integration system 1300.

### Under Table System

The UV air treatment unit of the present disclosure can provide scalable treatment systems for alternative mitigations. For example, instead of being mounted in a ceiling, in one embodiment, the UV air treatment fixture is mounted under a table. The scaling of these systems and associated operating methods allow for compact and effective pathogen reduction. The system mounted under the table can have multiple ducts and vents.

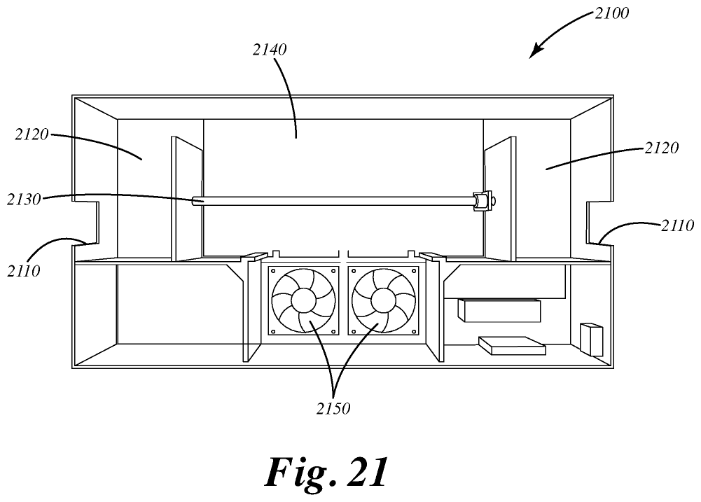

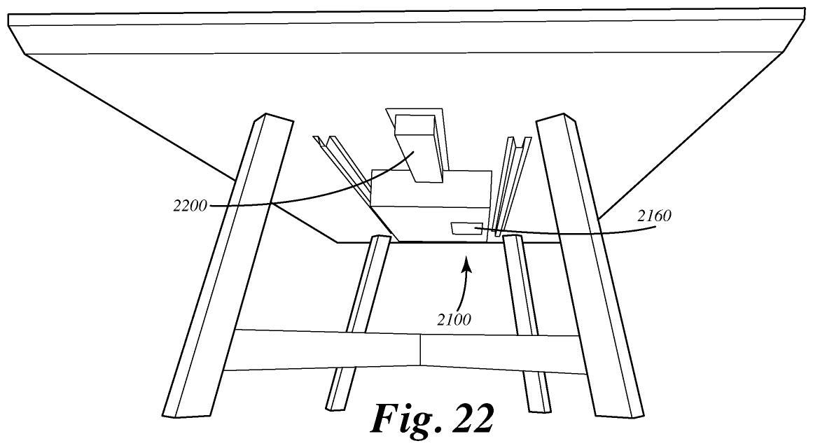

FIG. 21 shows a table mountable air treatment unit configuration. It includes two ducts 2110 to feed the reactor while allowing these ducts to be centerline of a table. Additional ducts can be added depending on the shape or configuration of the table taking into account individual seating arrangements. Air intake is taken from the table surface, treated, and then vented at knee level below the table. FIG. 21 depicts a top view of a table mounted air treatment system 2100 according to one embodiment. The air treatment system 2100 can include an inlet duct cutout 2110. The inlet duct cutout may be in fluid communication with an inlet duct interface 2120. The inlet duct interface 2120 can route airflow from the inlet duct cutout 2110. The air treatment system 2100 may include a UV source 2130. The air treatment system 2100 can have a diffuse reflector 2140 in the reactor. The air may flow through the fans 2150 and out the outlet 2160 (pictured in FIG. 22.) The air treatment system 2100 may also include electronics 2170.

FIG. 22 is the table-mounted air treatment system of FIG. 21 mounted to a table. The ducting 2200 allows inlets along the table surface with just holes in the surface enabling inlet vents at any point along the center duct plane.

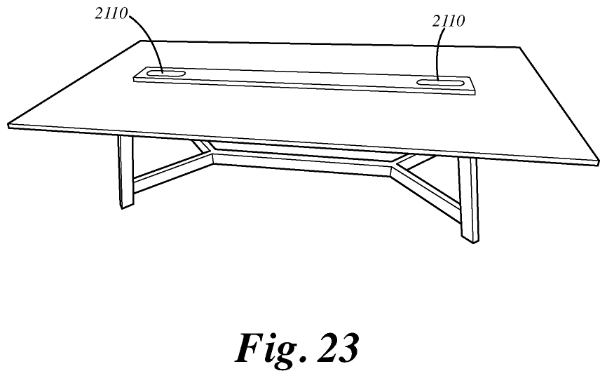

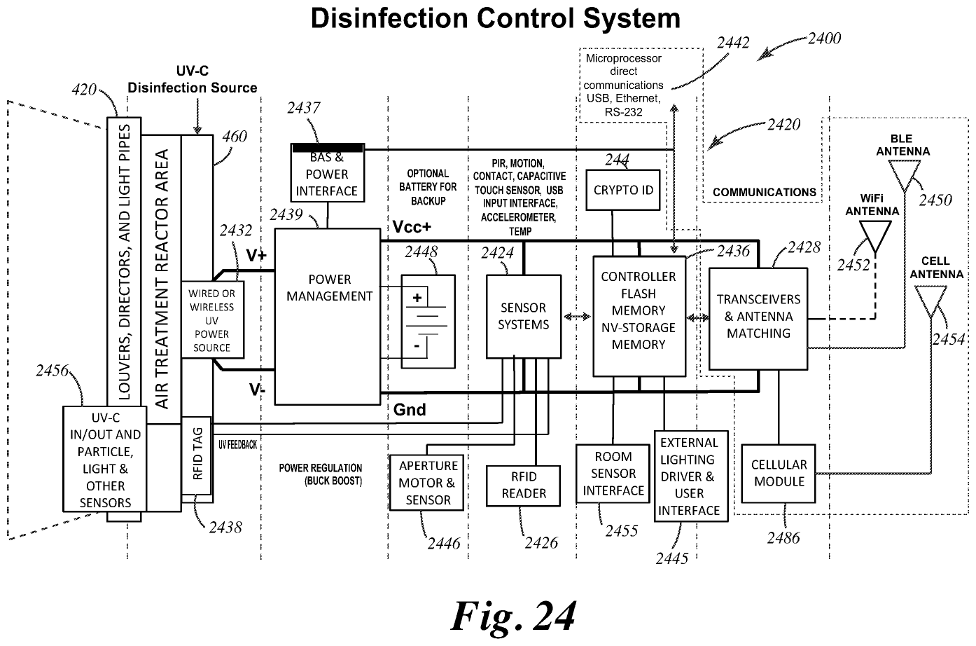

FIG. 23 is a top view of the table of FIG. 22. The vents also have an indicator to show treatment status. In one embodiment, the indicator may be an LED status indicator. The system can count people at the table with multiple occupancy sensors counting the people at and around the table.

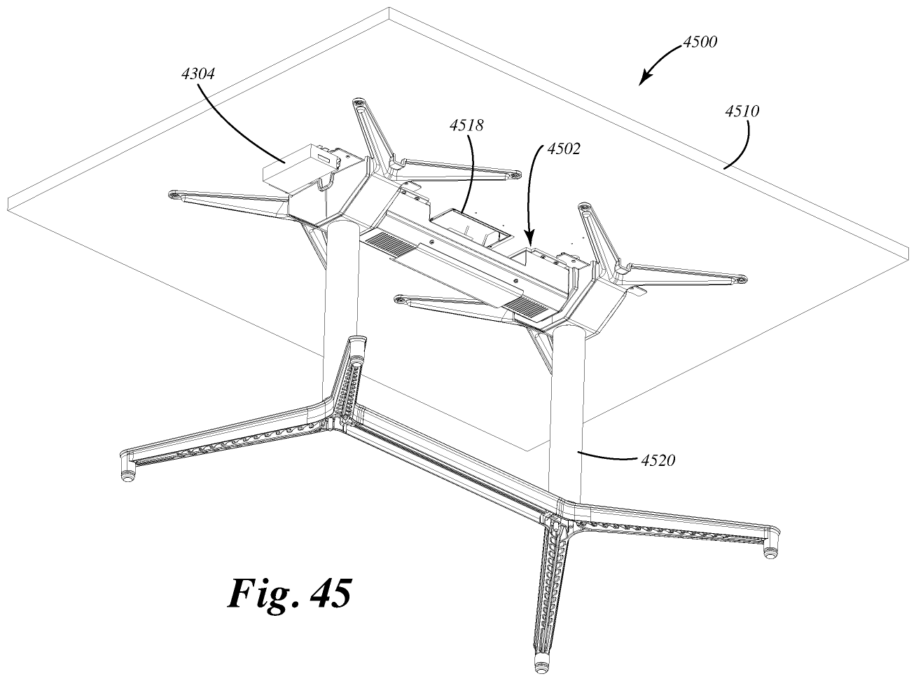

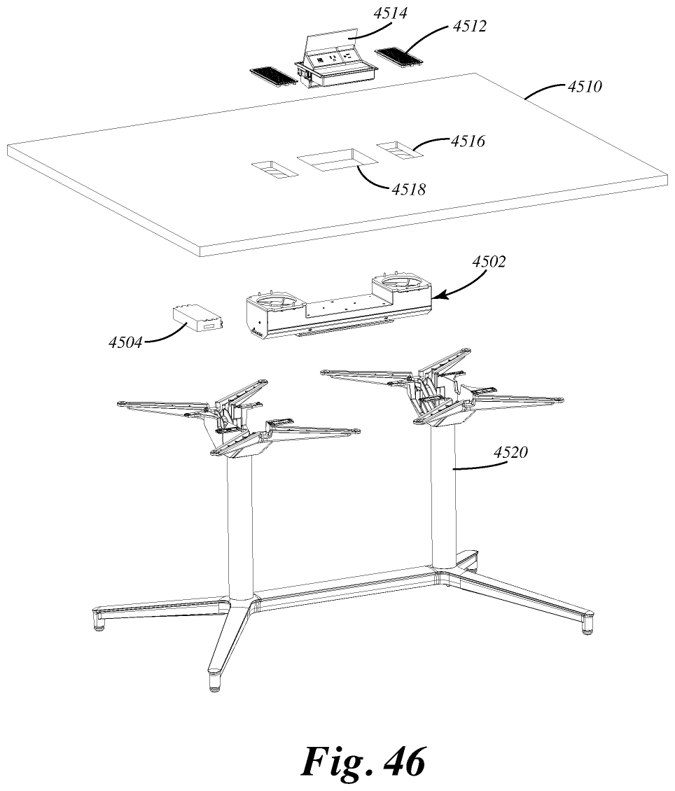

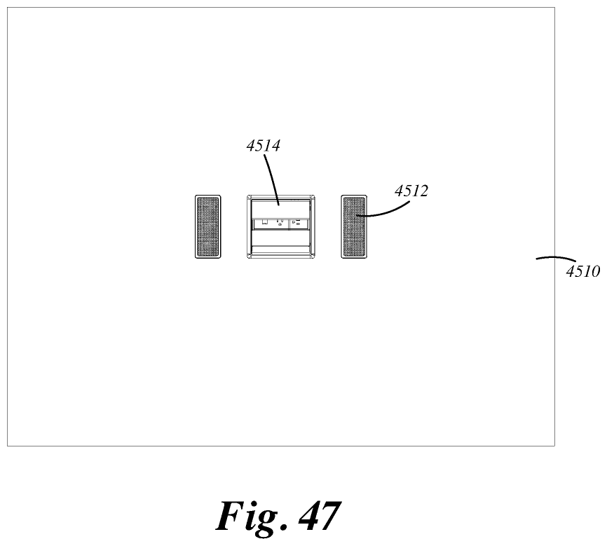

FIGS. 45-47 illustrate an alternative under table UV air treatment system 4500. In this system, a UV air under table assembly 4502 is mounted to the bottom surface of a table top 4510. The table top 4510 (and components of the UV air under table system 4500) can be supported by support legs 4520 or other supports. The tabletop can include air inlet ducts 4516 for receiving air to be treated by the UV air under table system 4500. The ducts 4516 may be covered by an aesthetic plate or grille 4512 along with a pre-filter (not shown) that snugly fits over top of the duct opening to provide a clean aesthetic look to the table surface. The table may or may not include one or more power/connection module apertures 4518 for seating one or more power/connection modules 4514 in the table top 4510 to provide power and/or connectivity to users sitting at the table. A UV air under table power module 4504 can be mounted to the underside of the table top 4510 and electrically connected to the UV air under table assembly to provide power and driving of various components, such as, for example, fans and UV bulbs within the UV air under table system housing 4530. The power module 4504 can be connected to a power source provided by the table or another power source in proximity.

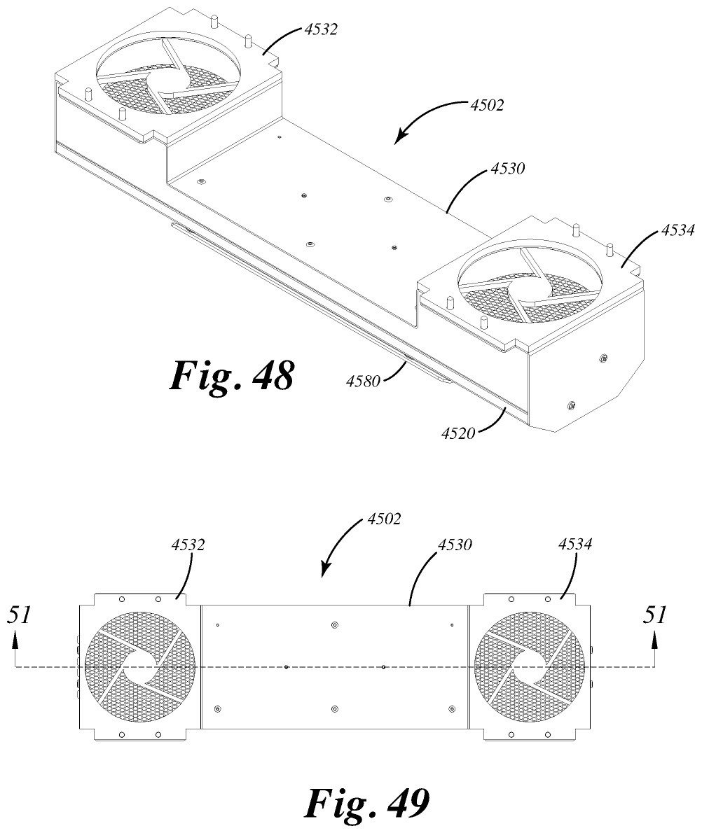

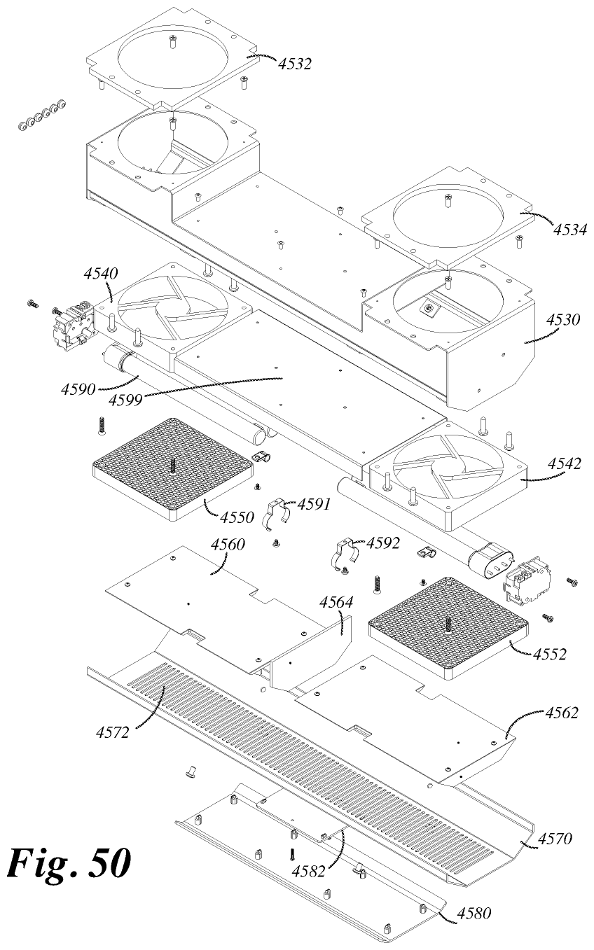

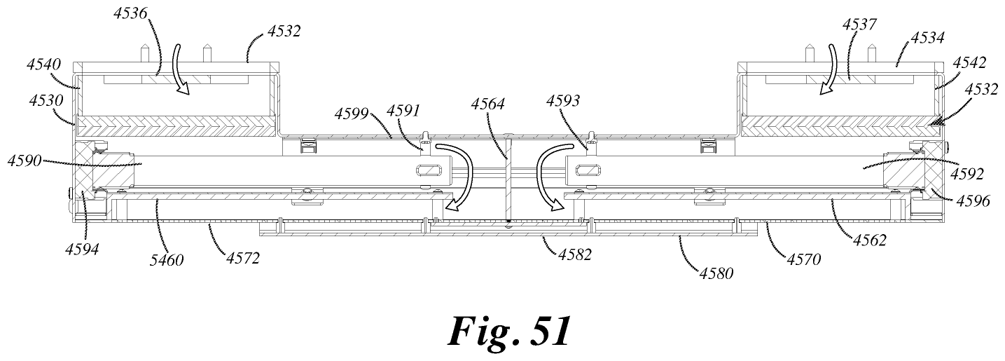

FIGS. 48-51 illustrate various views of the UV air treatment assembly 4500. Referring to the exploded view of FIG. 50, the assembly 4500 includes a main upper housing 4530, intake fans 4540, 4542, UV sources 4590, 4592, UV reducer airflow directors 4550, 4552, UV reflectors 4562, a main lower housing 4570, and a UV light blocking plates 4580, 4582. The UV air treatment assembly 4500 can be secured to the underside of the table with the two inlet plates 4532, 4534. The UV bulbs 4590, 4592 can be installed into respective UV bulb sockets 4594, 4596 and held in place with the UV bulb clips 4591, 4592.

Referring to the sectional view of FIG. 51, operation of the UV air treatment assembly 4500 will now be described. The fans 4540, 4542 are configured to draw air in through the respective inlets 4536, 4537, which is then routed through the tortured path created by the UV reducer airflow directors 4530, 4532. The air then reaches their respective UV air chambers where the UV sources 4590, 4592 treat the air as it flows through their respective chambers. In the current embodiment, a divider wall 4564 creates two mini UV reactor chambers. The fans push the air through the UV air chamber and out the vent outlets 4572 in the bottom of the assembly 4500. The UV light is largely blocked by the blocking plates, 4582, 4582, which also assist with dispersing airflow at the outlet along its length.

The UV reducer airflow directors 4530, 4532 serve a similar purpose as those described above in connection with the ceiling mounted UV air treatment system. In this embodiment, the UV reducer airflow directors 4530, 4532 prevent UV light from escaping out the inlets in the table. That is, while air is being drawn into the UV chamber, light bouncing around in the chamber (e.g., against the reflective surfaces 4560, 4562, 4564, 4599) likely will be directed toward the inlet. The UV reducer airflow directors will absorb UV light as it bounces through the columnar walls. Due to the angles of the UV reducer airflow directors, the amount of UV light (if any) that reaches the inlet will be substantially reduced. FIG. 51 illustrates that a v-shaped UV reducer airflow directors are installed in the current embodiment, but in alternative embodiments different types of UV reducer airflow directors can be utilized.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

The above description is that of current embodiments of the invention. Various alterations and changes can be made without departing from the spirit and broader embodiments of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular.

1. A UV air treatment ceiling fixture for mitigating air pathogens within a room, the UV air treatment ceiling fixture comprising:
a support member operable to facilitate mounting the fixture to a ceiling;
a germicidal light source operable to generate UV light;
a UV reactor chamber having an untreated air inlet and a treated air outlet;
a UV reducer airflow director, wherein the UV reducer airflow director comprises an array of hollow columnar cells having cell walls that permit airflow while reducing UV light intensity of UV light from the UV reactor chamber as the UV light reflects against cell walls of the UV reducer airflow director;
a fan configured to generate a current of air from the untreated air inlet to the treated air outlet through the UV reactor chamber, wherein the UV reducer airflow director improves uniformity of airflow velocity in the UV reactor chamber.

2. The UV air treatment ceiling fixture of claim 1 wherein the UV reactor chamber includes a main UV reactor portion and an inlet-side auxiliary UV reactor portion disposed between the main UV reactor portion and the untreated air inlet, wherein the UV reducer airflow director is disposed in the inlet-side auxiliary UV reactor portion of the UV reactor chamber in proximity of the untreated air inlet.

3. The UV air treatment ceiling fixture of claim 2 wherein the UV air treatment ceiling fixture further comprises:
an additional UV reducer airflow director disposed in an opening between the main UV reactor portion and the auxiliary UV reactor portion, wherein the additional UV reducer airflow director comprises an array of hollow columnar cells having cell walls that permit airflow between the auxiliary UV reactor portion of the UV reactor chamber and the main UV reactor portion of the UV reactor chamber while reducing UV light from the UV reactor chamber as the UV light reflects against cell walls of the additional UV reducer airflow director; and
wherein the additional UV reducer airflow director further contributes to improving uniformity of airflow velocity in the UV reactor chamber.

4. The UV air treatment ceiling fixture of claim 3 wherein the the UV reducer airflow director is oriented at a first orientation and the additional UV reducer airflow director is oriented at a second orientation such that the columnar cells of the UV reducer airflow director are substantially perpendicular to the columnar cells of the additional UV reducer airflow director.

5. The UV air treatment ceiling fixture of claim 3 wherein the UV reducer airflow director is oriented at a first orientation and the additional UV reducer airflow director is oriented at a second orientation such that the columnar cells of the UV reducer airflow director are disposed at an offset angle to the columnar cells of the additional UV reducer airflow director, wherein the offset angle between the first inlet side UV reducer airflow director and the additional UV reducer airflow director contribute to improving uniformity of airflow velocity in the UV reactor chamber and contribute to reducing UV light intensity of UV light from the UV reactor chamber as the UV light reflects against cell walls of the first inlet side UV reducer airflow director and the additional UV reducer airflow director to reduce intensity of the UV light below a target threshold before exiting the UV air treatment system.

6. The UV air treatment ceiling fixture of claim 1 wherein the UV reactor chamber includes a main UV reactor portion and an outlet-side auxiliary UV reactor portion disposed between the main UV reactor portion and the treated air outlet, wherein the UV reducer airflow director is disposed in the outlet-side auxiliary UV reactor portion of the UV reactor chamber in proximity of the treated air outlet.

7. The UV air treatment ceiling fixture of claim 6 further comprises:
an additional UV reducer airflow director disposed in an opening between the main UV reactor portion and the outlet-side auxiliary UV reactor portion, wherein the additional UV reducer airflow director comprises an array of hollow columnar cells having cell walls that permit airflow from the auxiliary UV reactor portion of the UV reactor chamber to the main UV reactor portion of the UV reactor chamber while reducing UV light from the UV reactor chamber as the UV light reflects against cell walls of the additional UV reducer airflow director; and
wherein the additional UV reducer airflow director further contributes to improving uniformity of airflow velocity in the UV reactor chamber.

8. The UV air treatment ceiling fixture of claim 7 wherein the UV reducer airflow director is oriented at a first orientation and the additional UV reducer airflow director is oriented at a second orientation such that the columnar cells of the UV reducer airflow director are substantially perpendicular to the columnar cells of the additional UV reducer airflow director.

9. The UV air treatment ceiling fixture of claim 7 wherein the UV reducer airflow director is oriented at a first orientation and the additional UV reducer airflow director is oriented at a second orientation such that the columnar cells of the UV reducer airflow director are disposed at an offset angle to the columnar cells of the additional UV reducer airflow director, wherein the offset angle between the UV reducer airflow director and the additional UV reducer airflow director contribute to improving uniformity of airflow velocity in the UV reactor chamber and contribute to reducing UV light intensity of UV light from the UV reactor chamber as the UV light reflects against cell walls of the UV reducer airflow director and the additional UV reducer airflow director to reduce intensity of the UV light below a target threshold before exiting the treated air outlet of the UV air treatment system.

10. The UV air treatment ceiling fixture of claim 1 includes a sensor configured to obtain occupancy information pertaining to how many occupants are present in the room, wherein the UV air treatment ceiling fixture is operable to dynamically control one or more of UV energy dosage in the UV reactor chamber and the fan based on the occupancy information.

11. The UV air treatment ceiling fixture of claim 1 comprising a control system operable to control operation of one or more of the germicidal light source and the fan, the control system including a wireless communication controller configured to transmit information to and receive information from an external network device.

12. The UV air treatment ceiling fixture of claim 11 wherein the information from the external network device can include occupancy information pertaining to how many occupants are present in the room, wherein the control system is operable to dynamically control one or more of the germicidal light source and the fan based on the occupancy information.

13.-20. (canceled)

21. An air treatment ceiling fixture comprising:
an air treatment body including:
a reactor chamber having an untreated air inlet, inlet chamber, main chamber portion, outlet chamber, and a treated air outlet, the reactor chamber including a germicidal light source operable to generate ultraviolet (UV) light, the reactor chamber having an air treatment region operable to receive air from the untreated air inlet and to direct air to the treated air outlet, wherein UV light from the germicidal light source is directed to the air treatment region;
the inlet chamber fluidly connected to the main chamber portion of the reactor chamber through the untreated air inlet, the inlet chamber defining an inlet chamber opening; and
an outlet chamber fluidly connected to the main chamber portion of the reactor chamber through the treated air outlet, the outlet chamber defining an outlet chamber opening, the outlet chamber including at least one UV reducer airflow director;
at least one UV reducer airflow director disposed in at least one of the inlet chamber and the outlet chamber, the at least one UV reducer airflow director comprises an array of hollow columnar cells having cell walls that permit airflow while reducing UV light intensity of UV light from the UV reactor chamber as the UV light reflects against cell walls of the UV reducer airflow director;
a fan configured to move air from the untreated air inlet to the treated air outlet of the reactor chamber; and
a plurality of vertical installation clips;
a configurable maintenance door for facilitating access to the air treatment body, the configurable maintenance door configured to span the reactor chamber opening, the inlet chamber opening, and the outlet chamber opening, the configurable maintenance door including:
a plurality of retention springs moveably coupling the configurable maintenance door to the air treatment body;
a functional door module;
an inlet configured to permit airflow into the inlet chamber through the inlet chamber opening, the inlet including at least one inlet opening; and
an outlet configured to permit airflow out of the outlet chamber through the outlet chamber opening, the outlet portion including at least one outlet opening;
wherein the fan generates an air stream between the at least one inlet opening and the at least one outlet opening.

22. The air treatment ceiling fixture of claim 21, wherein the plurality of retention springs are removable from the air treatment body without vertical access to the air treatment ceiling system.

23. The air treatment ceiling fixture of claim 22, wherein the configurable maintenance door is replaceable with a second configurable maintenance door.

24. The air treatment ceiling fixture of claim 21,
the air treatment body including a reactor chamber disconnection contact;
the reactor chamber defining a reactor chamber opening;
wherein the air treatment body includes a chamber access door including a chamber access door disconnection contact configured to connect with the reactor chamber disconnection contact in response to the chamber access door being moved to the seated position and to disconnect with in response to the chamber access door being moved to an unseated position,
wherein in response to the reactor chamber disconnection switch contact disconnecting with the chamber access door disconnection contact a power connection for the germicidal light source is interrupted, and
wherein in response to the the reactor chamber disconnection switch contact connecting with the chamber access door disconnection contact a power connection for the germicidal light source is completed.

25. The air treatment ceiling fixture of claim 21,
the inlet including at least one inlet louver defining the inlet opening, the inlet louver configured to direct airflow into the inlet chamber; and
the outlet including at least one outlet louver defining the outlet opening, the outlet louver configured to direct airflow out of the outlet chamber.

26. The air treatment ceiling fixture of claim 25, wherein the inlet louver and the outlet louver are configured to absorb UV light emitted from the UV light source.

27. The air treatment ceiling fixture of claim 25, wherein the inlet louver is configured at an angle relative to the inlet chamber opening, and wherein the outlet louver is configured at an angle relative to the outlet chamber opening such to increase UV light reflection against the inlet louver and outlet louver walls lowering UV light intensity exiting the air treatment ceiling fixture.

28. The air treatment ceiling system of claim 21, wherein the chamber access door includes a latch selectively securing the chamber access door to the air treatment body.

29.-81. (canceled)

82. The UV air treatment ceiling fixture of claim 1 including one or more sensors configured to obtain one or more of carbon dioxide (CO2) concentrations, volatile organic compound (VOC) levels, particulate matter concentrations, and ambient audio levels, wherein the UV air treatment ceiling fixture is operable to dynamically control one or more of UV energy dosage in the UV reactor chamber and the fan based on sensor readings.
